SEQUENCE LISTING (221 sequences)

SEQ ID NO: 1 - DNA, 14 bp, Artificial Sequence
  Description of Artificial Sequencehuman TGF-beta 1 antisense oligonucleotide
cgatagtcttgcag

SEQ ID NO: 2 - DNA, 14 bp, Artificial Sequence
  Description of Artificial Sequencehuman TGF-beta 1 antisense oligonucleotide
gtcgatagtcttgc

SEQ ID NO: 3 - DNA, 14 bp, Artificial Sequence
  Description of Artificial Sequencehuman TGF-beta 1 antisense oligonucleotide
cttggacaggatct

SEQ ID NO: 4 - DNA, 14 bp, Artificial Sequence
  Description of Artificial Sequencehuman TGF-beta 1 antisense oligonucleotide
ccaggaattgttgc

SEQ ID NO: 5 - DNA, 14 bp, Artificial Sequence
  Description of Artificial Sequencehuman TGF-beta 1 antisense oligonucleotide
cctcaatttcccct

SEQ ID NO: 6 - DNA, 14 bp, Artificial Sequence
  Description of Artificial Sequencehuman TGF-beta 1 antisense oligonucleotide
gatgtccacttgca

SEQ ID NO: 7 - DNA, 14 bp, Artificial Sequence
  Description of Artificial Sequencehuman TGF-beta 1 antisense oligonucleotide
ctccaaatgtaggg

SEQ ID NO: 8 - DNA, 14 bp, Artificial Sequence
  Description of Artificial Sequencehuman TGF-beta 1 antisense oligonucleotide
accttgctgtactg

SEQ ID NO: 9 - DNA, 14 bp, Artificial Sequence
  Description of Artificial Sequencehuman TGF-beta 1 antisense oligonucleotide
gtagtacacgatgg

SEQ ID NO: 10 - DNA, 14 bp, Artificial Sequence
  Description of Artificial Sequencehuman TGF-beta 1 antisense oligonucleotide
cacgtagtacacga

SEQ ID NO: 11 - DNA, 14 bp, Artificial Sequence
  Description of Artificial Sequencehuman TGF-beta 1 antisense oligonucleotide
catgttggacagct

SEQ ID NO: 12 - DNA, 14 bp, Artificial Sequence
  Description of Artificial Sequencehuman TGF-beta 1 antisense oligonucleotide
gcacgatcatgttg

SEQ ID NO: 13 - DNA, 16 bp, Artificial Sequence
  Description of Artificial Sequencehuman TGF-beta 1 antisense oligonucleotide
tgtactctgcttgaac

SEQ ID NO: 14 - DNA, 18 bp, Artificial Sequence
  Description of Artificial Sequencehuman TGF-beta 1 antisense oligonucleotide
ctgatgtgttgaagaaca

SEQ ID NO: 15 - DNA, 16 bp, Artificial Sequence
  Description of Artificial Sequencehuman TGF-beta 1 antisense oligonucleotide
ctctgatgtgttgaag

SEQ ID NO: 16 - DNA, 16 bp, Artificial Sequence
  Description of Artificial Sequencehuman TGF-beta 1 antisense oligonucleotide
ggaagtcaatgtacag

SEQ ID NO: 17 - DNA, 18 bp, Artificial Sequence
  Description of Artificial Sequencehuman TGF-beta 1 antisense oligonucleotide
catgtcgatagtcttgca

SEQ ID NO: 18 - DNA, 18 bp, Artificial Sequence
  Description of Artificial Sequencehuman TGF-beta 1 antisense oligonucleotide
agctgaagcaatagttgg

SEQ ID NO: 19 - DNA, 18 bp, Artificial Sequence
  Description of Artificial Sequencehuman TGF-beta 1 antisense oligonucleotide
gtcatagatttcgttgtg

SEQ ID NO: 20 - DNA, 18 bp, Artificial Sequence
  Description of Artificial Sequencehuman TGF-beta 1 antisense oligonucleotide
ctccacttttaacttgag

SEQ ID NO: 21 - DNA, 18 bp, Artificial Sequence
  Description of Artificial Sequencehuman TGF-beta 1 antisense oligonucleotide
tgctgtatttctggtaca

SEQ ID NO: 22 - DNA, 14 bp, Artificial Sequence
  Description of Artificial Sequencehuman TGF-beta 2 antisense oligonucleotide
cacacagtagtgca

SEQ ID NO: 23 - DNA, 14 bp, Artificial Sequence
  Description of Artificial Sequencehuman TGF-beta 2 antisense oligonucleotide
gcacacagtagtgc

SEQ ID NO: 24 - DNA, 17 bp, Artificial Sequence
  Description of Artificial Sequencehuman TGF-beta 2 antisense oligonucleotide
gcttgctcaggatctgc

SEQ ID NO: 25 - DNA, 14 bp, Artificial Sequence
  Description of Artificial Sequencehuman TGF-beta 2 antisense oligonucleotide
tactcttcgtcgct

SEQ ID NO: 26 - DNA, 14 bp, Artificial Sequence
  Description of Artificial Sequencehuman TGF-beta 2 antisense oligonucleotide
cttggcgtagtact

SEQ ID NO: 27 - DNA, 14 bp, Artificial Sequence
  Description of Artificial Sequencehuman TGF-beta 2 antisense oligonucleotide
gtaaacctccttgg

SEQ ID NO: 28 - DNA, 19 bp, Artificial Sequence
  Description of Artificial Sequencehuman TGF-beta 2 antisense oligonucleotide
gtctattttgtaaacctcc

SEQ ID NO: 29 - DNA, 20 bp, Artificial Sequence
  Description of Artificial Sequencehuman TGF-beta 2 antisense oligonucleotide
gcatgtctattttgtaaacc

SEQ ID NO: 30 - DNA, 18 bp, Artificial Sequence
  Description of Artificial Sequencehuman TGF-beta 2 antisense oligonucleotide
cggcatgtctattttgta

SEQ ID NO: 31 - DNA, 14 bp, Artificial Sequence
  Description of Artificial Sequencehuman TGF-beta 2 antisense oligonucleotide
ggcatcaaggtacc

SEQ ID NO: 32 - DNA, 14 bp, Artificial Sequence
  Description of Artificial Sequencehuman TGF-beta 2 antisense oligonucleotide
ctgtagaaagtggg

SEQ ID NO: 33 - DNA, 18 bp, Artificial Sequence
  Description of Artificial Sequencehuman TGF-beta 2 antisense oligonucleotide
acaattctgaagtagggt

SEQ ID NO: 34 - DNA, 18 bp, Artificial Sequence
  Description of Artificial Sequencehuman TGF-beta 2 antisense oligonucleotide
tcaccaaattggaagcat

SEQ ID NO: 35 - DNA, 20 bp, Artificial Sequence
  Description of Artificial Sequencehuman TGF-beta 2 antisense oligonucleotide
gctttcaccaaattggaagc

SEQ ID NO: 36 - DNA, 14 bp, Artificial Sequence
  Description of Artificial Sequencehuman TGF-beta 2 antisense oligonucleotide
ctggcttttgggtt

SEQ ID NO: 37 - DNA, 18 bp, Artificial Sequence
  Description of Artificial Sequencehuman TGF-beta 2 antisense oligonucleotide
tctgatatagctcaatcc

SEQ ID NO: 38 - DNA, 18 bp, Artificial Sequence
  Description of Artificial Sequencehuman TGF-beta 2 antisense oligonucleotide
tcctagtggactttatag

SEQ ID NO: 39 - DNA, 16 bp, Artificial Sequence
  Description of Artificial Sequencehuman TGF-beta 2 antisense oligonucleotide
tttttcctagtggact

SEQ ID NO: 40 - DNA, 19 bp, Artificial Sequence
  Description of Artificial Sequencehuman TGF-beta 2 antisense oligonucleotide
caattatcctgcacatttc

SEQ ID NO: 41 - DNA, 16 bp, Artificial Sequence
  Description of Artificial Sequencehuman TGF-beta 2 antisense oligonucleotide
gcaattatcctgcaca

SEQ ID NO: 42 - DNA, 16 bp, Artificial Sequence
  Description of Artificial Sequencehuman TGF-beta 2 antisense oligonucleotide
gcagcaattatcctgc

SEQ ID NO: 43 - DNA, 14 bp, Artificial Sequence
  Description of Artificial Sequencehuman TGF-beta 2 antisense oligonucleotide
tggcattgtaccct

SEQ ID NO: 44 - DNA, 14 bp, Artificial Sequence
  Description of Artificial Sequencehuman TGF-beta 2 antisense oligonucleotide
tgtgctgagtgtct

SEQ ID NO: 45 - DNA, 16 bp, Artificial Sequence
  Description of Artificial Sequencehuman TGF-beta 2 antisense oligonucleotide
cctgctgtgctgagtg

SEQ ID NO: 46 - DNA, 14 bp, Artificial Sequence
  Description of Artificial Sequencehuman TGF-beta 2 antisense oligonucleotide
cttgggtgttttgc

SEQ ID NO: 47 - DNA, 18 bp, Artificial Sequence
  Description of Artificial Sequencehuman TGF-beta 2 antisense oligonucleotide
tttagctgcatttgcaag

SEQ ID NO: 48 - DNA, 14 bp, Artificial Sequence
  Description of Artificial Sequencehuman TGF-beta 2 antisense oligonucleotide
gccacttttccaag

SEQ ID NO: 49 - DNA, 14 bp, Artificial Sequence
  Description of Artificial Sequencehuman TGF-beta 3 antisense oligonucleotide
tcgagcttccccca

SEQ ID NO: 50 - DNA, 14 bp, Artificial Sequence
  Description of Artificial Sequencehuman TGF-beta 3 antisense oligonucleotide
ccccgagcccaagg

SEQ ID NO: 51 - DNA, 13 bp, Artificial Sequence
  Description of Artificial Sequencehuman TGF-beta 3 antisense oligonucleotide
cccgacgagccgg

SEQ ID NO: 52 - DNA, 14 bp, Artificial Sequence
  Description of Artificial Sequencehuman TGF-beta 3 antisense oligonucleotide
acgcaccaaggcga

SEQ ID NO: 53 - DNA, 15 bp, Artificial Sequence
  Description of Artificial Sequencehuman TGF-beta 3 antisense oligonucleotide
cgggttgtcgagccc

SEQ ID NO: 54 - DNA, 13 bp, Artificial Sequence
  Description of Artificial Sequencehuman TGF-beta 3 antisense oligonucleotide
cggcagtgccccg

SEQ ID NO: 55 - DNA, 14 bp, Artificial Sequence
  Description of Artificial Sequencehuman TGF-beta 3 antisense oligonucleotide
cgcaattctgctcg

SEQ ID NO: 56 - DNA, 14 bp, Artificial Sequence
  Description of Artificial Sequencehuman TGF-beta 3 antisense oligonucleotide
ttcgttgtgctccc

SEQ ID NO: 57 - DNA, 14 bp, Artificial Sequence
  Description of Artificial Sequencehuman TGF-beta 3 antisense oligonucleotide
attccgactcggtg

SEQ ID NO: 58 - DNA, 17 bp, Artificial Sequence
  Description of Artificial Sequencehuman TGF-beta 3 antisense oligonucleotide
acgtgcgtcatcaccgt

SEQ ID NO: 59 - DNA, 10 bp, Artificial Sequence
  Description of Artificial Sequencehuman TGF-beta 3 antisense oligonucleotide
ccaagaagcc

SEQ ID NO: 60 - DNA, 14 bp, Artificial Sequence
  Description of Artificial Sequencehuman TGF-beta 3 antisense oligonucleotide
cctaatgccttcca

SEQ ID NO: 61 - DNA, 14 bp, Artificial Sequence
  Description of Artificial Sequencehuman TGF-beta 3 antisense oligonucleotide
tcagcagggccagg

SEQ ID NO: 62 - DNA, 17 bp, Artificial Sequence
  Description of Artificial Sequencehuman TGF-beta 3 antisense oligonucleotide
gcaaagttcagcagggc

SEQ ID NO: 63 - DNA, 16 bp, Artificial Sequence
  Description of Artificial Sequencehuman TGF-beta 3 antisense oligonucleotide
ggcaaagttcagcagg

SEQ ID NO: 64 - DNA, 18 bp, Artificial Sequence
  Description of Artificial Sequencehuman TGF-beta 3 antisense oligonucleotide
gtggcaaagttcagcagg

SEQ ID NO: 65 - DNA, 14 bp, Artificial Sequence
  Description of Artificial Sequencehuman TGF-beta 3 antisense oligonucleotide
gtggcaaagttcag

SEQ ID NO: 66 - DNA, 18 bp, Artificial Sequence
  Description of Artificial Sequencehuman TGF-beta 3 antisense oligonucleotide
gaccgtggcaaagttcag

SEQ ID NO: 67 - DNA, 15 bp, Artificial Sequence
  Description of Artificial Sequencehuman TGF-beta 3 antisense oligonucleotide
agagaggctgaccgt

SEQ ID NO: 68 - DNA, 17 bp, Artificial Sequence
  Description of Artificial Sequencehuman TGF-beta 3 antisense oligonucleotide
gagagagagaggctgac

SEQ ID NO: 69 - DNA, 15 bp, Artificial Sequence
  Description of Artificial Sequencehuman TGF-beta 3 antisense oligonucleotide
acagagagaggctga

SEQ ID NO: 70 - DNA, 15 bp, Artificial Sequence
  Description of Artificial Sequencehuman TGF-beta 3 antisense oligonucleotide
gtggacagagagagg

SEQ ID NO: 71 - DNA, 18 bp, Artificial Sequence
  Description of Artificial Sequencehuman TGF-beta 3 antisense oligonucleotide
caactggacagagagagg

SEQ ID NO: 72 - DNA, 16 bp, Artificial Sequence
  Description of Artificial Sequencehuman TGF-beta 3 antisense oligonucleotide
tcttcttgatgtggcc

SEQ ID NO: 73 - DNA, 17 bp, Artificial Sequence
  Description of Artificial Sequencehuman TGF-beta 3 antisense oligonucleotide
ccctcttcttcttgatg

SEQ ID NO: 74 - DNA, 14 bp, Artificial Sequence
  Description of Artificial Sequencehuman TGF-beta 3 antisense oligonucleotide
caccctcttcttct

SEQ ID NO: 75 - DNA, 17 bp, Artificial Sequence
  Description of Artificial Sequencehuman TGF-beta 3 antisense oligonucleotide
atggatttctttggcat

SEQ ID NO: 76 - DNA, 13 bp, Artificial Sequence
  Description of Artificial Sequencehuman TGF-beta 3 antisense oligonucleotide
ggatttctttggc

SEQ ID NO: 77 - DNA, 18 bp, Artificial Sequence
  Description of Artificial Sequencehuman TGF-beta 3 antisense oligonucleotide
aagttggactctcttctc

SEQ ID NO: 78 - DNA, 18 bp, Artificial Sequence
  Description of Artificial Sequencehuman TGF-beta 3 antisense oligonucleotide
taagttggactctcttct

SEQ ID NO: 79 - DNA, 16 bp, Artificial Sequence
  Description of Artificial Sequencehuman PGE antisense oligonucleotide
taggagtggttgaggc

SEQ ID NO: 80 - DNA, 17 bp, Artificial Sequence
  Description of Artificial Sequencehuman PGE antisense oligonucleotide
gtgtaggagtggttgag

SEQ ID NO: 81 - DNA, 14 bp, Artificial Sequence
  Description of Artificial Sequencehuman PGE antisense oligonucleotide
ctgtgtaggagtgg

SEQ ID NO: 82 - DNA, 14 bp, Artificial Sequence
  Description of Artificial Sequencehuman PGE antisense oligonucleotide
cccacatgcctgtg

SEQ ID NO: 83 - DNA, 14 bp, Artificial Sequence
  Description of Artificial Sequencehuman PGE antisense oligonucleotide
cgatgaacaacgag

SEQ ID NO: 84 - DNA, 16 bp, Artificial Sequence
  Description of Artificial Sequencehuman PGE antisense oligonucleotide
ctggcgatgaacaacg

SEQ ID NO: 85 - DNA, 14 bp, Artificial Sequence
  Description of Artificial Sequencehuman PGE antisense oligonucleotide
cgctggcgatgaac

SEQ ID NO: 86 - DNA, 15 bp, Artificial Sequence
  Description of Artificial Sequencehuman PGE antisense oligonucleotide
gagctagtcccgttg

SEQ ID NO: 87 - DNA, 15 bp, Artificial Sequence
  Description of Artificial Sequencehuman PGE antisense oligonucleotide
gcgaagagctagtcc

SEQ ID NO: 88 - DNA, 17 bp, Artificial Sequence
  Description of Artificial Sequencehuman PGE antisense oligonucleotide
ccagttatgcgaagagc

SEQ ID NO: 89 - DNA, 16 bp, Artificial Sequence
  Description of Artificial Sequencehuman PGE antisense oligonucleotide
ccccagttatgcgaag

SEQ ID NO: 90 - DNA, 14 bp, Artificial Sequence
  Description of Artificial Sequencehuman VEGF antisense oligonucleotide
cggccgcggtgtgt

SEQ ID NO: 91 - DNA, 17 bp, Artificial Sequence
  Description of Artificial Sequencehuman VEGF antisense oligonucleotide
cgggaatgcttccgccg

SEQ ID NO: 92 - DNA, 17 bp, Artificial Sequence
  Description of Artificial Sequencehuman VEGF antisense oligonucleotide
cggctcaccgcctcggc

SEQ ID NO: 93 - DNA, 14 bp, Artificial Sequence
  Description of Artificial Sequencehuman VEGF antisense oligonucleotide
cacgtctgcggatc

SEQ ID NO: 94 - DNA, 18 bp, Artificial Sequence
  Description of Artificial Sequencehuman VEGF antisense oligonucleotide
ccccgcatcgcatcaggg

SEQ ID NO: 95 - DNA, 14 bp, Artificial Sequence
  Description of Artificial Sequencehuman VEGF antisense oligonucleotide
cgccttgcaacgcg

SEQ ID NO: 96 - DNA, 14 bp, Artificial Sequence
  Description of Artificial Sequencehuman VEGF antisense oligonucleotide
ccgaccggggccgg

SEQ ID NO: 97 - DNA, 14 bp, Artificial Sequence
  Description of Artificial Sequencehuman VEGF antisense oligonucleotide
gttcatggtttcgg

SEQ ID NO: 98 - DNA, 15 bp, Artificial Sequence
  Description of Artificial Sequencehuman VEGF antisense oligonucleotide
gcagaaagttcatgg

SEQ ID NO: 99 - DNA, 14 bp, Artificial Sequence
  Description of Artificial Sequencehuman VEGF antisense oligonucleotide
gctgatagacatcc

SEQ ID NO: 100 - DNA, 14 bp, Artificial Sequence
  Description of Artificial Sequencehuman VEGF antisense oligonucleotide
gcgctgatagacat

SEQ ID NO: 101 - DNA, 16 bp, Artificial Sequence
  Description of Artificial Sequencehuman VEGF antisense oligonucleotide
gtagctgcgctgatag

SEQ ID NO: 102 - DNA, 14 bp, Artificial Sequence
  Description of Artificial Sequencehuman VEGF antisense oligonucleotide
ctcgatctcatcag

SEQ ID NO: 103 - DNA, 17 bp, Artificial Sequence
  Description of Artificial Sequencehuman VEGF antisense oligonucleotide
atgtactcgatctcatc

SEQ ID NO: 104 - DNA, 16 bp, Artificial Sequence
  Description of Artificial Sequencehuman VEGF antisense oligonucleotide
gaagatgtactcgatc

SEQ ID NO: 105 - DNA, 16 bp, Artificial Sequence
  Description of Artificial Sequencehuman VEGF antisense oligonucleotide
cttgaagatgtactcg

SEQ ID NO: 106 - DNA, 14 bp, Artificial Sequence
  Description of Artificial Sequencehuman VEGF antisense oligonucleotide
gcatcgcatcaggg

SEQ ID NO: 107 - DNA, 14 bp, Artificial Sequence
  Description of Artificial Sequencehuman VEGF antisense oligonucleotide
ccgcatcgcatcag

SEQ ID NO: 108 - DNA, 18 bp, Artificial Sequence
  Description of Artificial Sequencehuman VEGF antisense oligonucleotide
catttgttgtgctgtagg

SEQ ID NO: 109 - DNA, 18 bp, Artificial Sequence
  Description of Artificial Sequencehuman VEGF antisense oligonucleotide
ggtctgcattcacatttg

SEQ ID NO: 110 - DNA, 15 bp, Artificial Sequence
  Description of Artificial Sequencehuman VEGF antisense oligonucleotide
ctttggtctgcattc

SEQ ID NO: 111 - DNA, 15 bp, Artificial Sequence
  Description of Artificial Sequencehuman VEGF antisense oligonucleotide
ctttctttggtctgc

SEQ ID NO: 112 - DNA, 17 bp, Artificial Sequence
  Description of Artificial Sequencehuman VEGF antisense oligonucleotide
gctctatctttctttgg

SEQ ID NO: 113 - DNA, 17 bp, Artificial Sequence
  Description of Artificial Sequencehuman VEGF antisense oligonucleotide
gtcttgctctatctttc

SEQ ID NO: 114 - DNA, 16 bp, Artificial Sequence
  Description of Artificial Sequencehuman VEGF antisense oligonucleotide
cttgtcttgctctatc

SEQ ID NO: 115 - DNA, 18 bp, Artificial Sequence
  Description of Artificial Sequencehuman VEGF antisense oligonucleotide
catctgcaagtacgttcg

SEQ ID NO: 116 - DNA, 18 bp, Artificial Sequence
  Description of Artificial Sequencehuman VEGF antisense oligonucleotide
cacatctgcaagtacgtt

SEQ ID NO: 117 - DNA, 18 bp, Artificial Sequence
  Description of Artificial Sequencehuman VEGF antisense oligonucleotide
gtcacatctgcaagtacg

SEQ ID NO: 118 - DNA, 14 bp, Artificial Sequence
  Description of Artificial Sequencehuman VEGF antisense oligonucleotide
catctgcaagtacg

SEQ ID NO: 119 - DNA, 15 bp, Artificial Sequence
  Description of Artificial Sequencehuman VEGF antisense oligonucleotide
cacatctgcaagtac

SEQ ID NO: 120 - DNA, 14 bp, Artificial Sequence
  Description of Artificial Sequencehuman VEGF antisense oligonucleotide
gtcacatctgcaag

SEQ ID NO: 121 - DNA, 14 bp, Artificial Sequence
  Description of Artificial Sequencehuman VEGF antisense oligonucleotide
cttgtcacatctgc

SEQ ID NO: 122 - DNA, 16 bp, Artificial Sequence
  Description of Artificial Sequencehuman VEGF antisense oligonucleotide
ggcttgtcacatctgc

SEQ ID NO: 123 - DNA, 16 bp, Artificial Sequence
  Description of Artificial Sequencehuman VEGF antisense oligonucleotide
ctcggcttgtcacatc

SEQ ID NO: 124 - DNA, 14 bp, Artificial Sequence
  Description of Artificial Sequencehuman VEGF antisense oligonucleotide
ctccttcctcctgc

SEQ ID NO: 125 - DNA, 18 bp, Artificial Sequence
  Description of Artificial Sequencehuman VEGF antisense oligonucleotide
gcttgaagatgtacctcg

SEQ ID NO: 126 - DNA, 15 bp, Artificial Sequence
  Description of Artificial Sequencehuman VEGF antisense oligonucleotide
cgttgctctccgacg

SEQ ID NO: 127 - DNA, 18 bp, Artificial Sequence
  Description of Artificial Sequencehuman IL-10 antisense oligonucleotide
gtaaaactggatcatctc

SEQ ID NO: 128 - DNA, 18 bp, Artificial Sequence
  Description of Artificial Sequencehuman IL-10 antisense oligonucleotide
cttcttttgcaagtctgt

SEQ ID NO: 129 - DNA, 18 bp, Artificial Sequence
  Description of Artificial Sequencehuman IL-10 antisense oligonucleotide
tgagctgtgcatgccttc

SEQ ID NO: 130 - DNA, 15 bp, Artificial Sequence
  Description of Artificial Sequencehuman IL-10 antisense oligonucleotide
agtcaggaggaccag

SEQ ID NO: 131 - DNA, 15 bp, Artificial Sequence
  Description of Artificial Sequencehuman IL-10 antisense oligonucleotide
tgggtgccctggcct

SEQ ID NO: 132 - DNA, 15 bp, Artificial Sequence
  Description of Artificial Sequencehuman IL-10 antisense oligonucleotide
catgttaggcaggtt

SEQ ID NO: 133 - DNA, 17 bp, Artificial Sequence
  Description of Artificial Sequencehuman IL-10 antisense oligonucleotide
aggcatctcggagatct

SEQ ID NO: 134 - DNA, 16 bp, Artificial Sequence
  Description of Artificial Sequencehuman IL-10 antisense oligonucleotide
aaagtcttcactctgc

SEQ ID NO: 135 - DNA, 17 bp, Artificial Sequence
  Description of Artificial Sequencehuman IL-10 antisense oligonucleotide
aacaagttgtccagctg

SEQ ID NO: 136 - DNA, 15 bp, Artificial Sequence
  Description of Artificial Sequencehuman IL-10 antisense oligonucleotide
catcacctcctccag

SEQ ID NO: 137 - DNA, 18 bp, Artificial Sequence
  Description of Artificial Sequencehuman IL-10 antisense oligonucleotide
gggtcttcaggttctccc

SEQ ID NO: 138 - DNA, 18 bp, Artificial Sequence
  Description of Artificial Sequencehuman IL-10 antisense oligonucleotide
cacggccttgctcttgtt

SEQ ID NO: 139 - DNA, 18 bp, Artificial Sequence
  Description of Artificial Sequencehuman IL-10 antisense oligonucleotide
ttattaaaggcattcttc

SEQ ID NO: 140 - DNA, 18 bp, Artificial Sequence
  Description of Artificial Sequencehuman IL-10 antisense oligonucleotide
aagatgtcaaactcactc

SEQ ID NO: 141 - DNA, 18 bp, Artificial Sequence
  Description of Artificial Sequencehuman IL-10 antisense oligonucleotide
gtagttgatgaagatgtc

SEQ ID NO: 142 - DNA, 16 bp, Artificial Sequence
  Description of Artificial Sequencehuman IL-10 antisense oligonucleotide
gattttggagacctct

SEQ ID NO: 143 - DNA, 16 bp, Artificial Sequence
  Description of Artificial Sequencehuman IL-10 antisense oligonucleotide
tcagctatcccagagc

SEQ ID NO: 144 - DNA, 15 bp, Artificial Sequence
  Description of Artificial Sequencehuman IL-10 antisense oligonucleotide
ggctgggtcagctat

SEQ ID NO: 145 - DNA, 18 bp, Artificial Sequence
  Description of Artificial Sequencehuman IL-10 antisense oligonucleotide
aaatcgttcacagagaag

SEQ ID NO: 146 - DNA, 18 bp, Artificial Sequence
  Description of Artificial Sequencehuman IL-10 antisense oligonucleotide
tctttctaaatcgttcac

SEQ ID NO: 147 - DNA, 2745 bp, Artificial Sequence
  Description of Artificial Sequenceantisense mRNA of human TGF-beta 1
ctgcagccttgacctcccaggatcaagtgatcctcccaccttagcctccagagtagctgggaccacaggtgtacattttttaaaagtgttttgtagagatagggtctcactatgttacccaggctggtctcaaatgcctggattcaagtatcctcccatctctgcctcccaaaagtgctaggattacaggcgtgagcaccccgcctggcctgaactactatcttttattgtcttcttcactatcccccactaaagcaggttcctggtgggcaggaactcctcccttaacctctctgggcttgtttcctcaacctttaaaatgggtgttatcagagtccctgccatctcagagtgttgctatggtgactgaatgagttcattaatgtaaggcacttcaacagtgcccaaggtgctcaataaatagatctaactacagtagtgttccccactggtcccctgtgccttgatgccgggcaaaggaatagtgcagacaggcaggaggaggcagagagggagagagagggagtgggagtgggggaacgtcagggatggagaccccaggcaggcgcccaatgacacagagatccgcagtcctctctccatctttaatggggccccaggtgggcttggggcacggtgtccttaaatacagcccccatgggcaaggcagcgggggcggggcggggtggggccgggcctgccggggcggggcggggcggggcgggacctcagctgcacttgcaggagcgcacgatcatgttggacagctgctccaccttgggcttgcggcccacgtagtacacgatgggcagcggctccagcgcctgcggcacgcagcacggcgccgccgaggcgcccgggttatgctggttgtacagggccaggaccttgctgtactgcgtgtccaggctccaaatgtaggggcagggcccgaggcagaagttggcatggtagcccttgggctcgtggatccacttccagccgaggtccttgcggaagtcaatgtacagctgccgcacgcagcagttcttctccgtggagctgaagcaatagttggtgtccagggctcggcggtgccgggagctttgcagatgctgggccctctccagcggggtggccatgagaagcaggaaaggccggttcatgccatgaatggtggccaggtcacctcggcggccggtagtgaacccgttgatgtccacttgcagtgtgttatccctgctgtcacaggagcagtgggcgctaaggcgaaagccctcaatttcccctccacggctcaaccactgccgcacaactccggtgacatcaaaagataaccactctggcgagtcgctgggtgccagcagccggttgctgaggtatcgccaggaattgttgctgtatttctggtacagctccacgtgctgctccacttttaacttgagcctcctcagcagacgcagctctgcccgggagagcaacacgggttcaggtaccgcttctcggagctctgatgtgttgaagaacatatatatgctgtgtgtactctgcttgaacttgtcatagatttcgttgtgggtttccaccattagcacgcgggtgacctccttggcgtagtagtcggcctcaggctcgggctccggttctgcactctccccggccacccggtcgcgggtgctgttgtacagggcgagcacggcctcgggcagcgggccgggcggcacctccccctggctcggggggctggcgagccgcagcttggacaggatctggccgcggatggcctcgatgcgcttccgcttcaccagctccatgtcgatagtcttgcaggtggatagtcccgcggccggcgggccaggcgtcagcaccagtagccacagcagcggtagcagcagcggcagcagccgcagcccggagggcggcatgggggaggcggcgccccccggcactgccgagagcgcgaacagggctggtgtggtggggaggccccgcccctgcaggggctgggggtctcccggcaaaaggtaggagggcctcgagggaaagctgaggctcctcagggagaagggcgcagtggtggaggggaggcttggaccgggggtgtctcagtatcccacggaaataacctagatgggcgcgatctggtaccagaaggtgggtggtcttgaataggggatctgtggcaggtcggagagagatccgtctcctggaggagaaagggtctaggatgcgcgggggctcaggagacaggccggggatgaaggcggcgtgcagggggtgcgcccgaggtctggggaaaagtctttgcgggaggccgggtcggcgactcccgagggctggtccggaatgggggcgcctgagggacgccgtgtagggggcagggagggagcaagcgtccccggcggcaaagggaggcggtctggggtccccaagtcctgcctcctcgcggggcagcgtcgcgccaagaggtccccgcgcctccggctcccagcggcaacggaaaagtctcaaaagtttttttcctcttctcccgaccagctcgtccctcctcccgctcctcctccccctcctccccgcagtggcgggggcggcggcggctcgtctcagactctggggcctcaggctgctcctcggcgactccttcctccgctccgggccgaggccggccccgcgggcggctcagagccggggggggtgccccggacggggcgtcccccctgcccccggccggggccctcgctgtctggctgctccgcggagggaggt

SEQ ID NO: 148 - DNA, 1695 bp, Artificial Sequence
  Description of Artificial Sequenceantisense mRNA of human TGF-beta 2
tttaaaaaaatttgcttcttgtctctctcacttacaaagtaggtgaaatgtagaataaggccttcaactttttttgtgtcagatgccagttttaacaaacagaacacaaacttccaaagtgtctgaactagtaccgccttttcaaaaattttttaacactgatgaaccaaggctctcttatgttttcttgttacaagcatcatcgttgtcgtcgtcatcatcattatcatcatcattgtcattttggtcttgccacttttccaagaattttagctgcatttgcaagactttacaatcatattagaaagctgttcaatcttgggtgttttgccaatgtagtagagaatggttagaggttctaaatcttgggacacgcagcaaggagaagcagatgcttctggatttatggtattatataagctcaggaccctgctgtgctgagtgtctgaactccataaatacgggcatgctccagcacagaagttggcattgtaccctttgggttcgtgtatccatttccaccctagatccctcttgaaatcaatgtaaagtggacgtaggcagcaattatcctgcacatttctaaagcaataggccgcatccaaagcacgcttcttccgccggttggtctgttgtgactcaagtctgtaggagggcaataacattagcaggagatgtggggtcttcccactgttttttttcctagtggactttatagttttctgatcaccactggtatatgtggaggtgccatcaatacctgcaaatcttgcttctagttcttcacttttatttgggatgatgtaattattagatggtacaaaagtgcagcagggacagtgtaagcttattttaaatcccaggttcctgtctttatggtgaagccattcatgaacagcatcagttacatcgaaggagagccattcgccttctgctcttgttttcacaactttgctgtcgatgtagcgctgggttggagatgttaaatctttggacttgagaatctgatatagctcaatccgttgttcaggcactctggcttttgggttctgcaaacgaaagactctgaactctgctttcaccaaattggaagcattcttctccattgctgagacgtcaaatcgaacaattctgaagtagggtctgtagaaagtgggcgggatggcattttcggaggggaagaagggcggcatgtctattttgtaaacctccttggcgtagtactcttcgtcgctcctctcgcgctcgcaggcggccgccctccggctcgccttctcctggagcaagtccctggtgctgttgtagatggaaatcacctccggggggacttcctcgggctcaggatagtcttctgggggactggtgagcttcagcttgctcaggatctgcccgcggatcgcctcgatcctcttgcgcatgaactggtccatatcgagtgtgctgcaggtagacaggctgagcgcgaccgtgaccagatgcaggatcagaaaagcgctcagcacacagtagtgcattttttaaaaaagtggaaaaaaaagttgtttttaaaagtcagaataaaaaaaaagaaatcaacaattctcaaagtatagatcaaggagagttgtttggttttttgttgttgttgtttgtttttgatgcgaaacttttgcaaacaatctagtcaatgcccaacagaaaaacgtatcctgcttg

SEQ ID NO: 149 - DNA, 2529 bp, Artificial Sequence
  Description of Artificial Sequenceantisense mRNA of human TGF-beta 3
caggatgccccaaaaatatttatttatacaaagattttgagagtaatattcatacttgtctttatacctcagtctatgcgtctggggccaagtcactgtgtggcacatgtcgagcttccccgaatgcctcacatgttgtcgcacctgcttccaggaacaccaaatgaacacagggtcttggaggggaagtgggggaagaacccataatgccccaaccctgcatggaaccacaatccagaaatgtgcatcctgacctggaaggcgtctaaccaagtgtccaaggggaaatatgatcgagggagaggtgagaggagggacccagaggcagacaggagagggttgatttccaccctttcttctgcgttcagcatatccaaaaggcccaatacagttgatgggccaggaactgcatgacctggattttctccctgtagtgacccacgatgttaattgatgtagaggacagtttgcaaaagtaatagatttgcccttaatcccagacagtatgagatacaattctgggactttgtcttcgtaacctgtctttaaaaaaaaaaaaaaatgcttgccttgtataacataatccagattccctagagcagatgtggtacagcaatgagcaaatccaacctcagatctgaagtgtcttccagtctggccctgacccagccattctctgcccttccttctccctttagggtagcccaaatcccattgccacacaacatctcaacttaccatccctttcctctatccccatcccctctgtctgcgtcacagaaagtctgtgtgttctgaagagttcagccttcctctaaccaaacccacactttctttaccaccgtgattctcagagccagcaagaaagaaatgttccaaaaggaaacctccatctcagccatttgcccggagccgaaggttgtgggctccaggcctctcagtgaggtttgttgcttgtgtgtttcccgaggagcgggcagtcaggcagtggtggttctctctcccctctctctgtcgcacgtggggtctcagctacatttacaagacttcaccaccatgttggagagctgctccactttgggggtcctcccaacatagtacaggatggtcaggggctccaggtcctggggcacgcagcaaggcgaggcagatgcttcagggttcagagtgttgtacagtcccagcaccgtgctgtgggttgtgtctgcactgcggaggtatgggcaagggcctgagcagaagttggcatagtagcccttaggttcatggacccacttccagcccagatcctgtcggaagtcaatgtagagggggcgcacacagcagttctcctccaagttgcggaagcagtaattggtgtccaaagcccgcttcttcctctgacccccctggcccgggttgtcgagccggtgtgggggaatcatcatgaggattagatgagggttgtggtgatccttctgcttcttgaggcgccccagatctccacggccatggtcatcctcattgtccacgcctttgaatttgatttccatcacctcgtgaatgttttccaggatatctccattgggctgaaaggtgtgacatggacagtgaatgctgatttctagacctaagttggactctcttctcaacagccactcacgcacagtgtcagtgacatcaaaggacagccactcggcagtgccccgtgtgggcagattcttgccaccgatatagcgctgtttggcaatgtgctcatctggccgaaggatctggaagagctcgatcctctgctcattccgcttagagctggggttgggcacccgcaagacccggaattctgctcggaataggttggttctatttttctccactgaggacacattgaagcggaaaaccttggaggtaattcctttagggcagacagccagttcgttgtgctccgccagcccctggatcatgtcgaatttatggatttctttggcatagtattccgactcggtgttttcctgggtgcagccttcctccctctccccatgcatctcctccagcagctcccgggtgctgttgtaaagggccaggacctgataggggacgtgggtcatcaccgttggctcaggggggctggtgagcctgagcttgctcaagatctgtcccctaatggcttccaccctcttcttcttgatgtggccgaagtccaaggtggtgcaagtggacagagagaggctgaccgtggcaaagttcagcagggccaggaccaccagagccctttgcaagtgcatcttcatgtgtgagctgggaagagaggccagggggacggcaaggcctggagaggaagagaccccagcagacgtgcagaaggagggaggaaaaccaggcggcctccccagatcccaaagactgaggcttggcaagaaggtgcatgaactcactgcactgcgagagcttcaggacttccaggaagcgctggcaaccctgaggacgaagaagcggactgtgtgccttgtagcgctgggattcttgtccatgtgtctaaacaggttttgctgg

SEQ ID NO: 150 - DNA, 1259 bp, Artificial Sequence
  Description of Artificial Sequenceantisense mRNA of human IL-10
tcaccctatggaaacagcttaaaaacaggtgaaaataataaatattgaaaaaaattataatattgggcttctttctaaatcgttcacagagaagctcagtaaataaatagaaatgggggttgaggtatcagaggtaataaatattctataagagaggtacaataaggtttctcaaggggctgggtcagctatcccagagccccagatccgattttggagacctctaatttatgtcctagagtctatagagtcgccaccctgatgtctcagtttcgtatcttcattgtcatgtaggcttctatgtagttgatgaagatgtcaaactcactcatggctttgtagatgcctttctcttggagcttattaaaggcattcttcacctgctccacggccttgctcttgttttcacagggaagaaatcgatgacagcgccgtagcctcagcctgagggtcttcaggttctcccccagggagttcacatgcgccttgatgtctgggtcttggttctcagcttggggcatcacctcctccaggtaaaactggatcatctcagacaaggcttggcaacccaggtaacccttaaagtcctccagcaaggactcctttaacaacaagttgtccagctgatccttcatttgaaagaaagtcttcactctgctgaaggcatctcggagatctcgaagcatgttaggcaggttgcctgggaagtgggtgcagctgttctcagactgggtgccctggcctgggctggccctcaccccagtcaggaggaccaggcaacagagcagtgctgagctgtgcatgccttcttttgcaagtctgtcttgtggtttggttttgcaagagcaaccccctgatgtgtagaccttcacctctctgtcccccttttatattgtaagctcagggaggcctcttcattcattaaaaagccacaatcaaggtttcccggcacaggattttttctgcttagagctcctccttctctaacctctctaataaacttagttttcaatttttgcatcgtaagcaaaaatgattggttgaacatgaacttctgcattacagctatttttaggatgggctacctctcttagaataattttttagcttctcaattaaaaaaagttgatttcctggggagaacagctgttctgtccgcagaggccctcagctgtgggttctcattcgcgtgttcctaggtcacagtgacgtggacaaattgcccattccagaatacaatgggattgagaaataattgg

SEQ ID NO: 151 - DNA, 1765 bp, Artificial Sequence
  Description of Artificial Sequenceantisense mRNA of human Prostaglandin E2 Synthase
ccatgagatgcctgccatgacaggcgccacaaacctttcctttattgcaaacatgtcccagtcccgggaggcttgggaagagtgggaaccaggggaacccagggatgggattccactgaaaacaaaccgtcctgctgtcctgtcgagggcccccacccacaggatgtagccatgggacagccactgagggtccaggaagaggggcggcagagcagggaggcagggacagggaggggtcgccccagggcagtggcagggctggaactcgtccctaacatccctgagccccagcaggtgccctgtgttagaagcgagagggctggtgggggtgcgtggacaaggggcagaatgatcctgcccccaaccagtatcgccaggcgctggcccagtggccccaggccctggcagctggcgtcttccgctgccttccctctgctctgcgcggggacattcagtgcgctggggaggcctcggtgatggccctctccacccgcaggtaccagggctggatgtgcgtgtgctgcatcaggtcatcgaatgcatccagcccctccatcacacgcagcacgccatacaccgccaaatcagcgagattcggcttctggccccccatgaagggccggtccttgcccacagcagccacccacttgtcagcagcctcatagaggtcctcgcgcacgttgtcctggaggcggtgcctgctcttgagtcgcttgctgatgaggtacatggccgctgcacccatgtacttggccacggcaccctccacggctccgaacttgccctcgcggacaatgtagtcaaaggacgccagagcctcggtgggcgtgcggtacacattgggggagatcaggtgcaccagccagtcgtccgcccactgccgccacttcatctcctccgtcctggcctccttcccaccatacacttgctgggcctccttctcgttgagcatgagccagtacttattgccgaactcggtcacctccttgccctgctcgttcacagccttcatggctgggtagtaggtgatgatctcttccaggggctgccccgacaccaggtaggtcttgagggcgctgatgatgacagaggagtcatttagttgttgcgagctttctccttcctgggccaccaggatgggcacctttctgtaggaggagaacttgatctcagccctgcgcacagggttcacctccaccacctggtagggcagggcatggaagtcgaggaaggctcggaccttgctgcagaagggacacgtcttgtactggtacagggtcagctgcaggcggctggacagggagagctgcgcggctgagcgctctgcgtggaggtcctgggcgcgcaggtgccaccgcgccgtgtggtacagccccagggctccccccagggccagcgccgcagctcccagcagccgcgggctccccttacgagctgcagccacggggctcgggccgcccgccgcccccgcgaagccagcccggctctgcgtgggtagcagcggctgggggcggcctcccagcctccaggccaaggcgcacccaccaggccacagcgcccgcaccacccgcgcagccgggtccatgttcgctccgccggcgccgcgggcgggcgcgcgaaacgaagacgccgaggcacgcgcggcgtttaaagggccaggactctggcgccccgcgggttggccggggtgagggcgacgctaagggaaccctcagcgctctcgggactgggcgtgtgcccggcgcccaagttcgaaacgcccgcc

SEQ ID NO: 152 - DNA, 990 bp, Artificial Sequence
  Description of Artificial Sequenceantisense mRNA of human VEGF
cagtgtgctggcggccgcggtgtgtctacaggaatcccagaaataaaactctctaatcttccgggctcggtgatttagcagcaagaaaaataaaatggcgaatccaattccaagagggaccgtgctgggtcacccgcccgggaatgcttccgccggagtctcgccctccggacccaaagtgctctgcgcagagtctcctcttccttcatttcaggtttctggattaaggactgttctgtcgatggtgatggtgtggtggcggcagcgtggtttctgtatcgatcgttctgtatcagtctttcctggtgagagatctggttcccgaaaccctgagggaggctccttcctcctgcccggctcaccgcctcggcttgtcacatctgcaagtacgttcgtttaactcaagctgcctcgccttgcaacgcgagtctgtgtttttgcaggaacatttacacgtctgcggatcttgtacaaacaaatgctttctccgctctgagcaaggcccacagggattttcttgtcttgctctatctttctttggtctgcattcacatttgttgtgctgtaggaagctcatctctcctatgtgctggccttggtgaggtttgatccgcataatctgcatggtgatgttggactcctcagtgggcacacactccaggccctcgtcattgcagcagcccccgcatcgcatcaggggcacacaggatggcttgaagatgtactcgatctcatcagggtactcctggaagatgtccaccagggtctcgattggatggcagtagctgcgctgatagacatccatgaacttcaccacttcgtgatgattctgccctcctccttctgccatgggtgcagcctgggaccacttggcatggtggaggtagagcagcaaggcgaggctccaatgcacccaagacagcagaaagttcatggtttcggaggcccgaccggggccgggccggctcgcgccgggccgccagcacactg

SEQ ID NO: 153 - protein, 390 aa, Homo sapiens
MPPSGLRLLLLLLPLLWLLVLTPGRPAAGLSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 154 - protein, 20 aa, Artificial Sequence
  Description of Artificial Sequencepeptide fragments of human TGF-beta 1
ALDTNYCFSSTEKNCCVRQL

SEQ ID NO: 155 - protein, 20 aa, Artificial Sequence
  Description of Artificial Sequencepeptide fragments of human TGF-beta 1
YIDFRKDLGWKWIHEPKGYH

SEQ ID NO: 156 - protein, 20 aa, Artificial Sequence
  Description of Artificial Sequencepeptide fragments of human TGF-beta 1
ANFCLGPCPYIWSLDTQYSK

SEQ ID NO: 157 - protein, 20 aa, Artificial Sequence
  Description of Artificial Sequencepeptide fragments of human TGF-beta 1
VLALYNQHNPGASAAPCCVP

SEQ ID NO: 158 - protein, 20 aa, Artificial Sequence
  Description of Artificial Sequencepeptide fragments of human TGF-beta 1
QALEPLPIVYYVGRKPKVEQ

SEQ ID NO: 159 - protein, 12 aa, Artificial Sequence
  Description of Artificial Sequencepeptide fragments of human TGF-beta 1
LSNMIVRSCKCS

SEQ ID NO: 160 - protein, 20 aa, Artificial Sequence
  Description of Artificial Sequencepeptide fragments of human TGF-beta 1
TEKNCCVRQLYIDFRKDLGW

SEQ ID NO: 161 - protein, 20 aa, Artificial Sequence
  Description of Artificial Sequencepeptide fragments of human TGF-beta 1
KWIHEPKGYHANFCLGPCPY

SEQ ID NO: 162 - protein, 19 aa, Artificial Sequence
  Description of Artificial Sequencepeptide fragments of human TGF-beta 1
WSLDTQYSKVLALYNQHNP

SEQ ID NO: 163 - protein, 20 aa, Artificial Sequence
  Description of Artificial Sequencepeptide fragments of human TGF-beta 1
GASAAPCCVPQALEPLPIVY

SEQ ID NO: 164 - protein, 22 aa, Artificial Sequence
  Description of Artificial Sequencepeptide fragments of human TGF-beta 1
YVGRKPKVEQLSNMIVRSCKCS

SEQ ID NO: 165 - protein, 40 aa, Artificial Sequence
  Description of Artificial Sequencepeptide fragments of human TGF-beta 1
QYSKVLALYNQHNPGASAAPCCVPQALEPLPIVYYVGRKP

SEQ ID NO: 166 - protein, 40 aa, Artificial Sequence; identical to SEQ ID NO: 165
  Description of Artificial Sequencepeptide fragments of human TGF-beta 1

SEQ ID NO: 167 - protein, 112 aa, Artificial Sequence
  Description of Artificial Sequencepeptide fragments of human TGF-beta 1
ALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 168 - protein, 30 aa, Artificial Sequence
  Description of Artificial Sequencepeptide fragments of human TGF-beta 1
ALDTNYCFSSTEKNCCVRQLYIDFRKDLGW

SEQ ID NO: 169 - protein, 30 aa, Artificial Sequence
  Description of Artificial Sequencepeptide fragments of human TGF-beta 1
KWIHEPKGYHANFCLGPCPYIWSLDTQYSK

SEQ ID NO: 170 - protein, 30 aa, Artificial Sequence
  Description of Artificial Sequencepeptide fragments of human TGF-beta 1
VLALYNQHNPGASAAPCCVPQALEPLPIVY

SEQ ID NO: 171 - protein, 22 aa, Artificial Sequence
  Description of Artificial Sequencepeptide fragments of human TGF-beta 1
YVGRKPKVEQLSNMIVRSCKCS

SEQ ID NO: 172 - protein, 30 aa, Artificial Sequence
  Description of Artificial Sequencepeptide fragments of human TGF-beta 1
CVRQLYIDFRKDLGWKWIHEPKGYHANFCL

SEQ ID NO: 173 - protein, 30 aa, Artificial Sequence
  Description of Artificial Sequencepeptide fragments of human TGF-beta 1
GPCPYIWSLDTQYSKVLALYNQHNPGASAA

SEQ ID NO: 174 - protein, 30 aa, Artificial Sequence
  Description of Artificial Sequencepeptide fragments of human TGF-beta 1
PCCVPQALEPLPIVYYVGRKPKVEQLSNMI

SEQ ID NO: 175 - protein, 413 aa, Homo sapiens
MHYCVLSAFLILHLVTVALSLSTCSTLDMDQFMRKRIEAIRGQILSKLKLTSPPEDYPEPEEVPPEVISIYNSTRDLLQEKASRRAAACERERSDEEYYAKEVYKIDMPPFFPSENAIPPTFYRPYFRIVRFDVSAMEKNASNLVKAEFRVFRLQNPKARVPEQRIELYQILKSKDLTSPTQRYIDSKVVKTRAEGEWLSFDVTDAVHEWLHHKDRNLGFKISLHCPCCTFVPSNNYIIPNKSEELEARFAGIDGTSTYTSGDQKTIKSTRKKNSGKTPHLLLMLLPSYRLESQQTNRRKRALDAAYCFRNVQDNCCLRPLYIDFKRDLGWKWIHEPKGYNANFCAGACPYLWSSDTQHSRVLSLYNTINPEASASPCCVSQDLEPLTILYYIGKTPKIEQLSNMIVKSCKCS

SEQ ID NO: 176 - protein, 20 aa, Artificial Sequence
  Description of Artificial Sequencepeptide fragments of human TGF-beta 2
ALDAAYCFRNVQDNCCLRPL

SEQ ID NO: 177 - protein, 20 aa, Artificial Sequence
  Description of Artificial Sequencepeptide fragments of human TGF-beta 2
YIDFKRDLGWKWIHEPKGYN

SEQ ID NO: 178 - protein, 20 aa, Artificial Sequence
  Description of Artificial Sequencepeptide fragments of human TGF-beta 2
ANFCAGACPYLWSSDTQHSR

SEQ ID NO: 179 - protein, 20 aa, Artificial Sequence
  Description of Artificial Sequencepeptide fragments of human TGF-beta 2
VLSLYNTINPEASASPCCVS

SEQ ID NO: 180 - protein, 20 aa, Artificial Sequence
  Description of Artificial Sequencepeptide fragments of human TGF-beta 2
QDLEPLTILYYIGKTPKIEQ

SEQ ID NO: 181 - protein, 12 aa, Artificial Sequence
  Description of Artificial Sequencepeptide fragments of human TGF-beta 2
LSNMIVKSCKCS

SEQ ID NO: 182 - protein, 20 aa, Artificial Sequence
  Description of Artificial Sequencepeptide fragments of human TGF-beta 2
VQDNCCLRPLYIDFKRDLGW

SEQ ID NO: 183 - protein, 20 aa, Artificial Sequence
  Description of Artificial Sequencepeptide fragments of human TGF-beta 2
KWIHEPKGYNANFCAGACPY

SEQ ID NO: 184 - protein, 20 aa, Artificial Sequence
  Description of Artificial Sequencepeptide fragments of human TGF-beta 2
LWSSDTQHSRVLSLYNTINP

SEQ ID NO: 185 - protein, 20 aa, Artificial Sequence
  Description of Artificial Sequencepeptide fragments of human TGF-beta 2
EASASPCCVSQDLEPLTILY

SEQ ID NO: 186 - protein, 22 aa, Artificial Sequence
  Description of Artificial Sequencepeptide fragments of human TGF-beta 2
YIGKTPKIEQLSNMIVKSCKCS

SEQ ID NO: 187 - protein, 41 aa, Artificial Sequence
  Description of Artificial Sequencepeptide fragments of human TGF-beta 2
QHSRVLSLYNTINPEASASPCCVSQDLEPLTILYYIGKTPK

SEQ ID NO: 188 - protein, 41 aa, Artificial Sequence; identical to SEQ ID NO: 187
  Description of Artificial Sequencepeptide fragments of human TGF-beta 2

SEQ ID NO: 189 - protein, 112 aa, Artificial Sequence
  Description of Artificial Sequencepeptide fragments of human TGF-beta 2
ALDAAYCFRNVQDNCCLRPLYIDFKRDLGWKWIHEPKGYNANFCAGACPYLWSSDTQHSRVLSLYNTINPEASASPCCVSQDLEPLTILYYIGKTPKIEQLSNMIVKSCKCS

SEQ ID NO: 190 - protein, 30 aa, Artificial Sequence
  Description of Artificial Sequencepeptide fragments of human TGF-beta 2
ALDAAYCFRNVQDNCCLRPLYIDFKRDLGW

SEQ ID NO: 191 - protein, 30 aa, Artificial Sequence
  Description of Artificial Sequencepeptide fragments of human TGF-beta 2
KWIHEPKGYNANFCAGACPYLWSSDTQHSR

SEQ ID NO: 192 - protein, 30 aa, Artificial Sequence
  Description of Artificial Sequencepeptide fragments of human TGF-beta 2
VLSLYNTINPEASASPCCVSQDLEPLTILY

SEQ ID NO: 193 - protein, 22 aa, Artificial Sequence
  Description of Artificial Sequencepeptide fragments of human TGF-beta 2
YIGKTPKIEQLSNMIVKSCKCS

SEQ ID NO: 194 - protein, 30 aa, Artificial Sequence
  Description of Artificial Sequencepeptide fragments of human TGF-beta 2
CLRPLYIDFKRDLGWKWIHEPKGYNANFCA

SEQ ID NO: 195 - protein, 30 aa, Artificial Sequence
  Description of Artificial Sequencepeptide fragments of human TGF-beta 2
GACPYLWSSDTQHSRVLSLYNTINPEASAS

SEQ ID NO: 196 - protein, 30 aa, Artificial Sequence
  Description of Artificial Sequencepeptide fragments of human TGF-beta 2
PCCVSQDLEPLTILYYIGKTPKIEQLSNMI

SEQ ID NO: 197 - protein, 412 aa, Homo sapiens
MKMHLQRALVVLALLNFATVSLSLSTCTTLDFGHIKKKRVEAIRGQILSKLRLTSPPEPTVMTHVPYQVLALYNSTRELLEEMHGEREEGCTQENTESEYYAKEIHKFDMIQGLAEHNELAVCPKGITSKVFRFNVSSVEKNRTNLFRAEFRVLRVPNPSSKRNEQRIELFQILRPDEHIAKQRYIGGKNLPTRGTAEWLSFDVTDTVREWLLRRESNLGLEISIHCPCHTFQPNGDILENIHEVMEIKFKGVDNEDDHGRGDLGRLKKQKDHHNPHLILMMIPPHRLDNPGQGGQRKKRALDAAYCFRNVQDNCCLRPLYIDFKRDLGWKWIHEPKGYNANFCAGACPYLWSSDTQHSRVLSLYNTINPEASASPCCVSQDLEPLTILYYIGKTPKIEQLSNMIVKSCKCS

SEQ ID NO: 198 - protein, 20 aa, Artificial Sequence
  Description of Artificial Sequencepeptide fragments of human TGF-beta 3
ALDTNYCFRNLEENCCVRPL

SEQ ID NO: 199 - protein, 20 aa, Artificial Sequence
  Description of Artificial Sequencepeptide fragments of human TGF-beta 3
YIDFRQDLGWKWVHEPKGYY

SEQ ID NO: 200 - protein, 20 aa, Artificial Sequence
  Description of Artificial Sequencepeptide fragments of human TGF-beta 3
ANFCSGPCPYLRSADTTHST

SEQ ID NO: 201 - protein, 20 aa, Artificial Sequence
  Description of Artificial Sequencepeptide fragments of human TGF-beta 3
VLGLYNTLNPEASASPCCVP

SEQ ID NO: 202 - protein, 20 aa, Artificial Sequence
  Description of Artificial Sequencepeptide fragments of human TGF-beta 3
QDLEPLTILYYVGRTPKVEQ

SEQ ID NO: 203 - protein, 12 aa, Artificial Sequence
  Description of Artificial Sequencepeptide fragments of human TGF-beta 3
LSNMVVKSCKCS

SEQ ID NO: 204 - protein, 20 aa, Artificial Sequence
  Description of Artificial Sequencepeptide fragments of human TGF-beta 3
NLEENCCVRPLYIDFRQDLG

SEQ ID NO: 205 - protein, 20 aa, Artificial Sequence
  Description of Artificial Sequencepeptide fragments of human TGF-beta 3
WKWVHEPKGYYANFCSGPCP

SEQ ID NO: 206 - protein, 20 aa, Artificial Sequence
  Description of Artificial Sequencepeptide fragments of human TGF-beta 3
YLRSADTTHSTVLGLYNTLN

SEQ ID NO: 207 - protein, 20 aa, Artificial Sequence
  Description of Artificial Sequencepeptide fragments of human TGF-beta 3
PEASASPCCVPQDLEPLTIL

SEQ ID NO: 208 - protein, 23 aa, Artificial Sequence
  Description of Artificial Sequencepeptide fragments of human TGF-beta 3
YYVGRTPKVEQLSNMVVKSCKCS

SEQ ID NO: 209 - protein, 41 aa, Artificial Sequence
  Description of Artificial Sequencepeptide fragments of human TGF-beta 3
THSTVLGLYNTLNPEASASPCCVPQDLEPLTILYYVGRTPK

SEQ ID NO: 210 - protein, 41 aa, Artificial Sequence; identical to SEQ ID NO: 209
  Description of Artificial Sequencepeptide fragments of human TGF-beta 3

SEQ ID NO: 211 - protein, 112 aa, Artificial Sequence; identical to SEQ ID NO: 189
  Description of Artificial Sequencepeptide fragments of human TGF-beta 3

SEQ ID NO: 212 - protein, 30 aa, Artificial Sequence; identical to SEQ ID NO: 190
  Description of Artificial Sequencepeptide fragments of human TGF-beta 3

SEQ ID NO: 213 - protein, 30 aa, Artificial Sequence; identical to SEQ ID NO: 191
  Description of Artificial Sequencepeptide fragments of human TGF-beta 3

SEQ ID NO: 214 - protein, 30 aa, Artificial Sequence; identical to SEQ ID NO: 192
  Description of Artificial Sequencepeptide fragments of human TGF-beta 3

SEQ ID NO: 215 - protein, 22 aa, Artificial Sequence
  Description of Artificial Sequencepeptide fragments of human TGF-beta 3
YIGKTPKIEQLSNMIVKSCKCS

SEQ ID NO: 216 - protein, 30 aa, Artificial Sequence; identical to SEQ ID NO: 194
  Description of Artificial Sequencepeptide fragments of human TGF-beta 3

SEQ ID NO: 217 - protein, 30 aa, Artificial Sequence; identical to SEQ ID NO: 195
  Description of Artificial Sequencepeptide fragments of human TGF-beta 3

SEQ ID NO: 218 - protein, 30 aa, Artificial Sequence; identical to SEQ ID NO: 196
  Description of Artificial Sequencepeptide fragments of human TGF-beta 3

SEQ ID NO: 219 - protein, 40 aa, Artificial Sequence; identical to SEQ ID NO: 165
  Description of Artificial Sequencepeptide fragments of human TGF-beta 1

SEQ ID NO: 220 - protein, 41 aa, Artificial Sequence; identical to SEQ ID NO: 187
  DISULFID (21): intermolecular disulfide bridge to SEQ ID No. 188

SEQ ID NO: 221 - protein, 41 aa, Artificial Sequence; identical to SEQ ID NO: 209
  Description of Artificial Sequencepeptide fragments of human TGF-beta 3

(19) United States
(12) Patent Application Publication
(10) Pub. No.: US 2010/0160208 A1
(43) Pub. Date: Jun. 24, 2010

(54) Pharmaceutical composition

(75) Inventors: Karl-Hermann Schlingensiepen, Donaustauf (DE); Reimar Schlinensiepen, Regensburg (DE)

Correspondence Address: JACOBSON HOLMAN PLLC, 400 SEVENTH STREET N.W., SUITE 600, WASHINGTON, DC, 20004, US

(73) Assignee: Antisense Pharma GmbH

(21) Appl. No.: 12/585,086

(22) Filed: Sep. 2, 2009

Related U.S. Application Data
(63) Continuation of application No. 10/581,547, filed on Jun. 2, 2006, now abandoned.
(60) Provisional application No. 60/541,771, filed on Feb. 5, 2004.

(30) Foreign Application Priority Data
Dec. 19, 2003 (EP) EP 03 029 367.4

(51) Int. Cl. A61K 38/00; A61K 31/7088; A61K 31/7125; A61K 31/712

(52) U.S. Cl. 514/2; 514/44 A

(57) ABSTRACT

The invention concerns a pharmaceutical composition comprising at least one stimulator of the immune cell functions and at least one substance inhibiting the cell proliferation and/or inducing cell death. In a preferred embodiment the stimulator of the function of the immune system and/or the immune cells are antagonists of TGF-beta selected from the group of oligonucleotides hybridizing with an area of the messenger RNA and or DNA encoding TGF-beta and the at least one substance inhibiting cell proliferation and/or inducing cell death is selected from the group of temozolomide, nitrosoureas, Vinca alkaloids, antagonists of the purine and pyrimidines bases, cytoststatic active antibiotics, caphthotecine derivatives, anti estrogens, anti-androgens and analogs of gonadotropin releasing hormone.

### FIELD OF THE INVENTION

This invention relates to pharmaceutical compositions and methods for treating neoplasms, in one preferred embodiment to neoplasms of the brain such as glioma, glioblastoma or astrocytoma.

Antineoplastic chemotherapeutic agents and radiation are the most common agents and methods, besides surgery for the treatment of neoplasms. Antineoplastic chemotherapeutic agents comprise e.g. alkylating agents, antimetabolites and alkaloids derived from plants. The common effect of these antineoplastic chemotherapeutic agents and radiation is the unspecific inhibition of the cell proliferation and the unspecific induction of cell death respectively, by a wide range of different mechanisms not completely discovered so far.

The inhibition of cell proliferation and the induction of cell death, respectively, primarily influence rapidly growing cells such as tumor cells. But at the same time the proliferation of other rapidly growing cells such as cells of the hair follicle, colon mucosa cells and also immune cells is inhibited. The immune cells inhibited are for example T-lymphocytes, B-lymphocytes, natural killer cells, granulocytes, macrophages, microglia cells as well as the respective precursor cells of the bone marrow. The administration of antineoplastic chemotherapeutic agents unspecifically inhibiting cell growth is therefore associated with severe side effects and general suppression of the function of the immune system, which has been proven by a lot of “in vitro” and “in vivo” results. For example, dacarbazine which cannot be clearly classified according to standard classification so far is reported to inhibit the humoral and cell-mediated immune response in mouse cells (Giampietri 1978, Nardelli 1984). The same results can be found for temozolomide which is an active metabolite of dacarbazine. Further “in vitro” studies of temozolomide show inhibition of cytotoxicity of lymphocyte activated killer cells (Alvino et al. 1999). CCNU (lomustine) as a representative for alkylating antineoplastic agents was shown to suppress both T-cells and B-cells (Bernego et al. 1984) by e.g. suppressing T-cell mediated cytotoxicity and further suppresses T-cell mediated cytotoxicity (Einstein et al. 1975). Further “in vitro” comparative studies of the alkylating agent cyclophosphamide, the antimetabolite 5-fluorouracil, the alkaloid vincristin and the antibiotic doxorubicine have in common to clearly show suppression of the cytotoxic T-cell function (Gereis et al. 1987).

Whereas the chemotherapy of some neoplasms is very successful, many neoplasms are accompanied by a poor life expectancy.

Another approach in the therapy of neoplasms is the stimulation of the immune system. There is a wide range of stimulators of the function of the immune system and/or the immune cells e.g. immune cell attracting substances, viruses and molecules involved in antigen processing, presentation or transporting, fusion cells of dendritic and tumor cells. Antagonists of substances downregulating the function of the immune system are regarded as stimulators of the immune system as well.

As a common principle these immune stimulators employ the ability of the immune system to selectively kill “foreign” tumor cells while sparing other fast growing “self” cells. This is of course a superior approach for treating neoplasms compared to unspecific inhibition of all growing cells or unspecific destruction of cells of an organism, respectively, which is the principle of the above mentioned antineoplastic chemotherapeutic agents as well as of radiation.

One example for a potent inhibitor of the immune system is TGF-beta (transforming growth factor-beta) mediating the neoplasms' escape from immunosurveillance (Wojtowicz-Praga, S. 1997). Cellular immunity is highly suppressed in patients suffering from neoplasms producing high levels of TGF-beta (de Visser, K. E. et al. 1999).

Using a substance specifically inhibiting the TGF-beta production and thus stimulating the immune system is a promising approach for the treatment of neoplasms (Stauder, G. et al. 2003).

Despite these promising results the tumor therapy with immunostimulators seems to have margins at least in very quick growing tumors.

Therefore there is an urgent need for the development of new therapeutics also for the treatment of fast growing neoplasms that are more reliable, have less side effects and increase the life spans of patients suffering from neoplasms.

In a clinical phase I/II study, upon administration of an immunostimulatory agent (antagonist of the immunosuppressor TGF-beta), we surprisingly recognized that the median overall survival of patients treated with an antineoplastic chemotherapeutic agent before the treatment with this immunostimulatory agent was clearly longer compared to patients not pre-treated with an antineoplastic chemotherapeutic agent.

Since the antineoplastic agents suppress the immune system by inhibiting the proliferation of the immunocompetent cells, as described above, up to now the approach of combining these antineoplastic agents with stimulators of the immune system in human beings were deemed not to be an appropriate approach for tumor therapy or tumor medicaments. In the literature reporting about neoplasm therapy by stimulation of the immune system it is emphasized that there has to be a sufficient time delay between the administration of a chemotherapeutic agent and a substance stimulating an immune response (e.g. Timmermann, J. M. 2002).

This inhibitory effect of chemotherapeutics on cells of the immune system also was proven in experiments. In these experiments cells of the immune system were treated with antineoplastic therapeutics and stimulators of the immune system, namely antisense oligonucleotides inhibiting TGF-beta. These assays are described in experiment 4 and the results are depicted in FIGS. 9 and 10.

### SUMMARY OF THE INVENTION

Surprisingly patients treated with a combination of an antineoplastic chemotherapeutic agent and a stimulator of the immune system according to this invention showed significant longer life spans compared to patients treated with either of these therapeutics.

The invention comprises a pharmaceutical composition with a stimulator of the function of the immune system and/or immune cells and substances inhibiting cell proliferation and/or inducing cell death.

In a preferred embodiment the invention is a pharmaceutical composition comprising at least one antagonist of TGF-beta and at least one antineoplastic chemotherapeutic agent. The at least two substances are mixed or are separate. The antagonist of TGF-beta is selected from the group of TGF-beta specific nucleotides, TGF-beta binding proteins that are no antibodies, TGF-beta binding receptors, parts of TGF-beta binding receptors, TGF-beta specific peptides and low molecular substances binding TGF-beta or any of their proteins, receptors, part of receptor protein or low molecular substance inhibiting the function of TGF-beta.

In yet another embodiment the TGF-beta antagonist is selected from the group of oligonucleotides hybridising with an area of the messenger RNA (m-RNA) and/or DNA encoding TGF-beta, TGF-beta receptors and/or parts of them binding TGF-beta, proteins, except antibodies, inhibiting TGF-beta peptides of less than 100 kDa inhibiting TGF-beta, peptides being parts of TGF-beta.

In yet a more preferred embodiment the TGF-beta specific nucleotides are antisense oligonucleotides against TGF-beta.

Another part of this invention are peptides that are part of TGF-beta, their use for the preparation of a pharmaceutical composition and the method of treating neoplasms with this preparation.

Methods to treat neoplasms are also part of this invention. The substances or methods stimulating the function of the immune system and/or the immune cells are administered with substances inhibiting cell proliferation and/or inducing cell death. The substances are administered by any known route in the art for administering medicaments.

The at least two substances of the pharmaceutical compositions according to this invention are mixed together or separately, optionally in the same carrier formulation or in separate pharmaceutical carriers.

The treatment of a patient suffering from unwanted neoplasms with a pharmaceutical composition as described above, in a preferred embodiment additionally with radiation is also part of this invention.

The at least two substances of the pharmaceutical compositions according to this invention are administered in parallel, in sequence, through the same route or different routes, together with the radiation, before or after the radiation.

Surprisingly patients suffering from neoplasms that were treated with at least one substance stimulating the immune system and/or the immune cells together with a substance inhibiting the cell proliferation and/or inducing cell death, and/or radiation show clearly longer life spans compared to patients treated with each of these medicaments and/or therapies alone.

This can lead to a reduction of the dosage of one of these medicaments and/or therapies being administered and thus to the reduction of potential undesirable side effects.

Another embodiment of this invention are peptides, that are part of TGF-beta. These peptides, their use for the preparation of a pharmaceutical composition, the use of this composition for the treatment of neoplasms, and the method of treating persons with neoplasms with those peptides is also part of this invention.

### FIGURES

FIG. 1 depicts a comparative study of lymphokine activated killer cell (LAK cell) mediated cytotoxicity on glioma cells. One part of the cells was incubated with the TGF-beta 2 specific antisense oligonucleotide with Seq. Id. No. 30, the other part was additionally treated with CCNU. The figure clearly points out that the cytotoxic activity of LAK cells treated with CCNU in combination with TGF-beta 2 specific antisense oligonucleotide with Seq. Id. No. 30 is superior compared to LAK cells treated with only TGF-beta 2 specific antisense oligonucleotide with Seq. Id. No. 30.

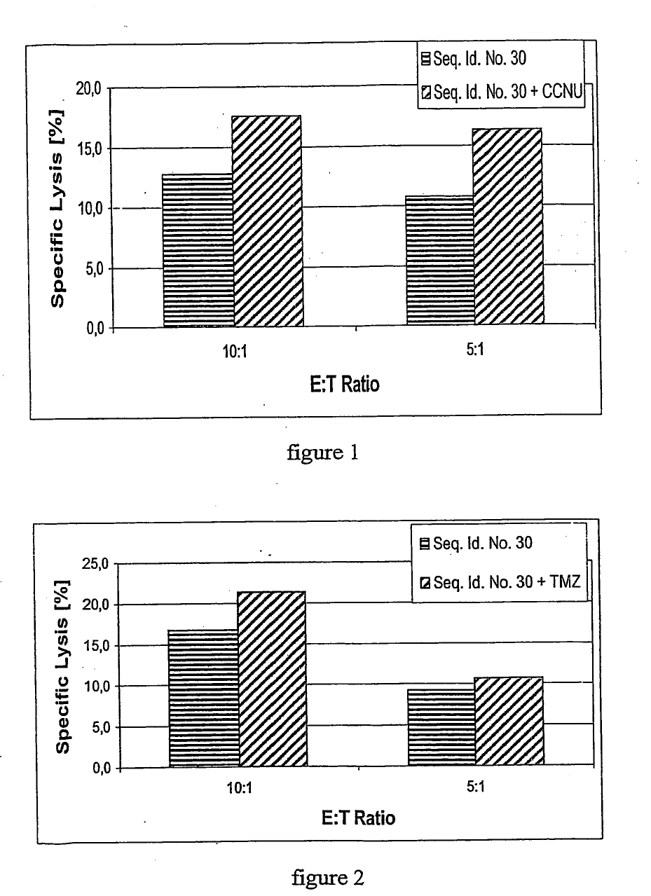

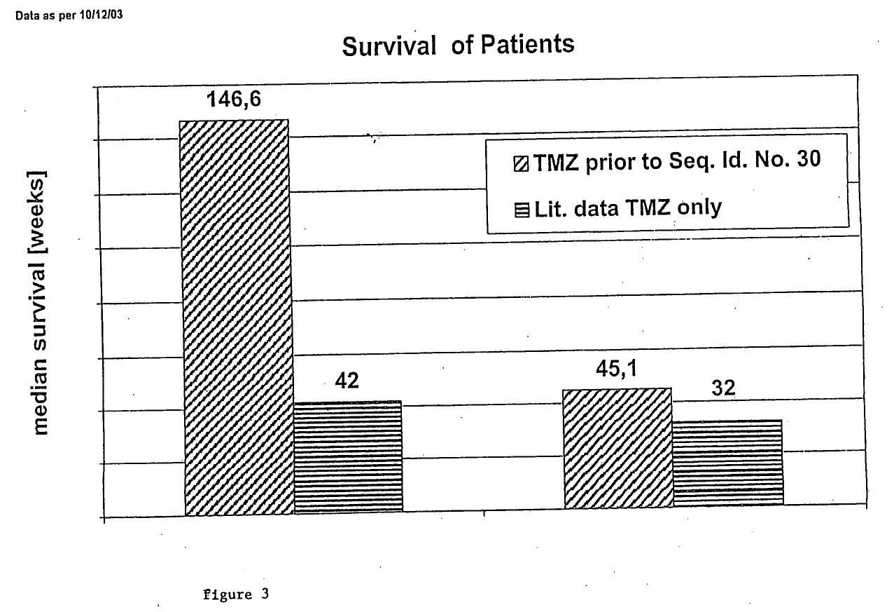

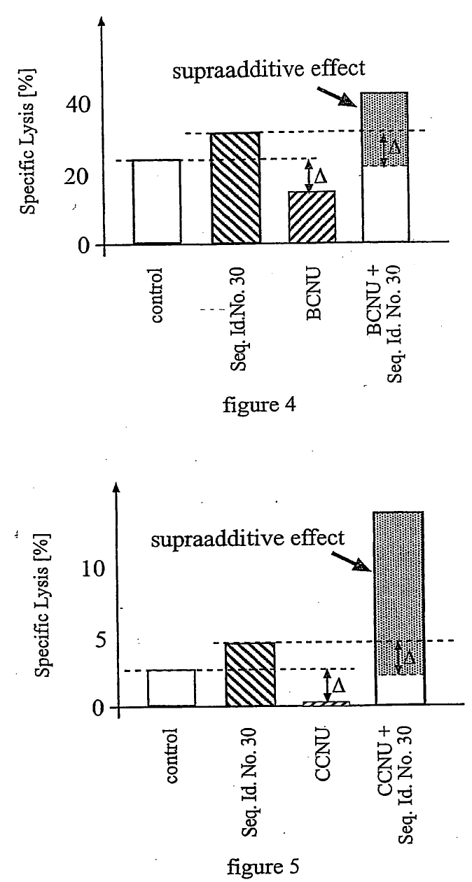

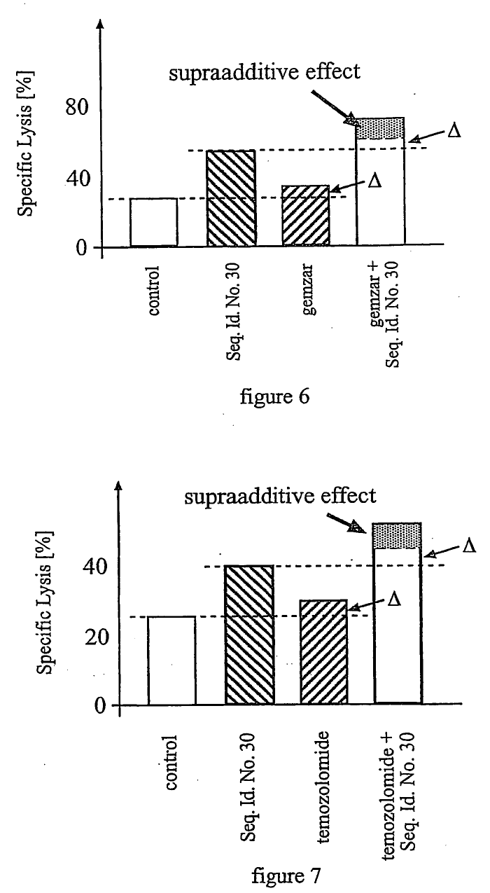

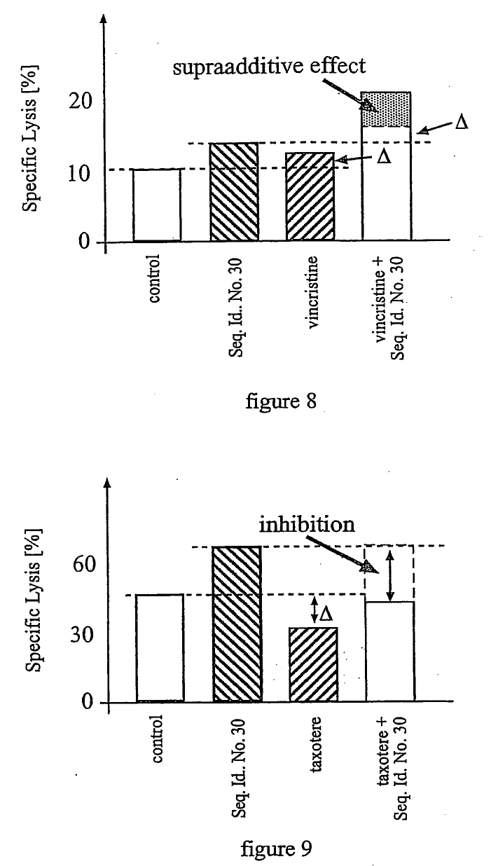

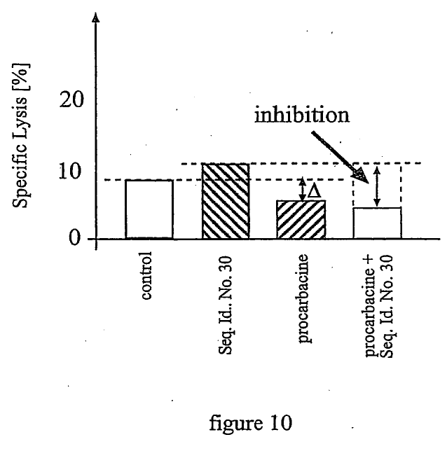

5×10⁶ PBMC (peripheral blood mononuclear cells) were cultivated in 4 μL RPMI 1640 medium supplemented with 10% foetal calf serum, in the presence of 10 ng/ml rh IL-2 (recombinant human interleukin 2), in 5% CO₂ atmosphere at 37° C. The first 3 days 5 μM TGF-beta 2 specific antisense oligonucleotide with Seq. Id. No. 30 was added. After that one part of the cells was incubated with 10 μM CCNU for an additional 6 h. Cell-mediated cytotoxicity, quantified by CARE-LASS assay (Lichtenfels et al. 1994), of LAK cells treated with TGF-beta 2 specific antisense oligonucleotide with Seq. Id. No. 30 (horizontal hachures) was compared to LAK cells treated with TGF-beta 2 specific antisense oligonucleotide with Seq. Id. No. 30 in combination with CCNU (diagonal hachures). Indicated are means±SD of quadruplicates.

FIG. 2 depicts a comparative study of lymphokine activated killer cell (LAK cell) mediated cytotoxicity on glioma cells. One part of the cells was incubated with the TGF-beta 2 specific antisense oligonucleotide with Seq. Id. No. 30 the other part was subsequently treated with Temozolomid (TMZ). The figure clearly points out that the cytotoxic activity of LAK cells treatment with temozolomid after the treatment with TGF-beta 2 specific antisense oligonucleotide with Seq. Id. No. 30 is superior compared to LAK cells treated only with TGF-beta 2 specific antisense oligonucleotide with Seq. Id. No. 30. 5×10⁶ LAK were cultivated in RPMI medium supplemented with 10% foetal calf serum, in the presence of 10 ng/ml rh IL-2 (recombinant human interleukin 2), in 5% CO₂ atmosphere at 37° C. The first 3 days 5 μM TGF-beta 2 specific antisense oligonucleotide with Seq. Id. No. 30 was added. Cell-mediated cytotoxicity was then quantified by CARE-LASS assay (Lichtenfels et al. 1994) in one part of the cells without further treatment (only TGF-beta 2 specific antisense oligonucleotide with Seq. Id. No. 30, horizontal hachures), in the other part in the presence of 30 μM temozolomid (TMZ, diagonal hachures). Indicated are means±SD of quadruplicates.

FIG. 3 depicts survival data of patients treated with the TGF-beta antisense oligonucleotide with Seq. Id. No. 30 after treatment with temozolomide according to standard schedule compared to the median overall survival time of patients treated with temozolomide only according to standard schedule. Survival time is given from start of first chemotherapy after tumor recurrence. Median overall survival time in the clinical study is evaluated from 3 patients with anaplastic astrocytoma and 10 patients with glioblastoma. The survival data are compared to the survival data of the literature. Our data reveal longer median overall survival times if applying TGF-beta 2 specific antisense oligonucleotide with Seq. Id. No. 30 following temozolomide than the comparable published data for temozolomide alone: 146.6 weeks vs. 42.0 weeks for patients suffering from anaplastic astrocytoma and 45.1 weeks versus 32.0 weeks for patients suffering from glioblastoma.

FIG. 4 depicts the specific lysis of tumor cells in an in vitro assay with A-172 cell line performed according to descriptions in example 7 in a ratio of effector cells to target cells of 20:1. Antisense oligonucleotide with Sequence Id. No. 30 was under test at a concentration of 5 micrMol enhancing cell lysis. In contrast BCNU at a concentration of 4 μM inhibited LAK cell induced cell lysis, which indicates its immunosuppressive effect. Surprisingly, the cytolytic effect of Seq. Id. No. 30 (5 microM) was enlarged supraadditively in combination with BCNU (4 μM) (specific cell lysis of control: 25.2%, BCNU 15.6%, Seq. Id. No. 30: 29.4%, Seq. Id. No. 30 in combination with BCNU: 40.7%).

FIG. 5 depicts the specific lysis of tumor cells in an in vitro assay with A-172 cell line performed according to descriptions in example 7 in a ratio of effector cells to target cells of 1.25:1. Antisense oligonucleotide with Sequence Id. No. 30 was under test at a concentration of 5 microMol enhancing cell lysis. In contrast CCNU at a concentration of 10 μM inhibited LAK cell induced cell lysis, which indicates its immunosuppressive effect. Surprisingly, the cytolytic effect of Seq. Id. No. 30 (5 microM) was enlarged supraadditively in combination with CCNU (10 μM) (specific cell lysis of control:2.6%, CCNU 0.5%, Seq. Id. No. 30: 4.4%, Seq. Id. No. 30 in combination with CCNU: 13.3%).

FIG. 6 depicts the specific lysis of tumor cells in an in vitro assay with Hup-T3 cell line performed according to descriptions in example 7 in a ratio of effector cells to target cells of 10:1. Antisense oligonucleotide with Sequence Id. No. 30 was under test at a concentration of 5 microM enhancing cell lysis. In contrast gemzar at a concentration of 20 μg/ml inhibited LAK cell induced cell lysis, which indicates its immunosuppressive effect. Surprisingly, the cytolytic effect of Seq. Id. No. 30 (5 microM) was enlarged supraadditively in combination with gemzar (20 μg/ml) (specific cell lysis of control: 32.9%, gemzar 34.5%, Seq. Id. No. 30: 59.5%, Seq. Id. No. 30 in combination with gemzar: 75.4%).

FIG. 7 depicts the specific lysis of tumor cells in an in vitro assay with A-172 cell line performed according to descriptions in example 7 in a ratio of effector cells to target cells of 10:1. Antisense oligonucleotide with Sequence Id. No. 30 was under test at a concentration of 5 microMol enhancing cell lysis. A very small increase of LAK cell induced cell lysis could be observed with temozolomide at a concentration of 50 μM. But, surprisingly, the cytolytic effect of Seq. Id. No. 30 (5 microM) was enlarged supraadditively in combination with temozolomide (50 μM) (specific cell lysis of control: 25.2%, temozolomide 31.3%, Seq. Id. No. 30: 39.2%, Seq. Id. No. 30 in combination with temozolomide: 50.4%).

FIG. 8 depicts the specific lysis of tumor cells in an in vitro assay with A-172 cell line performed according to descriptions in example 7 in a ratio of effector cells to target cells of 2.5:1. Antisense oligonucleotide with Sequence Id. No. 30 was under test at a concentration of 5 microMol enhancing cell lysis. A very small increase of LAK cell induced cell lysis could be observed with vincristine at a concentration of 0.04 μmol/ml. But, surprisingly, the cytolytic effect of Seq. Id. No. 30 (5 microM) was enlarged supraadditively in combination with vincristine (0.04 μmol/ml) (specific cell lysis of control: 10.1%, vincristine 12.6%, Seq. Id. No. 30: 13.9%, Seq. Id. No. 30 in combination with vincristine: 20.5%).

FIG. 9 depicts the specific lysis of tumor cells in an in vitro assay with NCL-H661 cell line performed according to descriptions in example 7 in a ratio of effector cells to target cells of 1.25:1. Antisense oligonucleotide with Sequence Id. No. 14 was under test at a concentration of 5 microMol enhancing cell lysis. In contrast taxotere reduced LAK induced cell lysis at a concentration of 0.37 μg/ml. But in this case the cytolytic effect of Seq. Id. No. 14 (5 microM) was reduced in combination with taxotere (0.37 μg/ml) (specific cell lysis of control: 49.6%, taxotere 30.5%, Seq. Id. No. 14: 65.3%, Seq. Id. No. 30 in combination with taxotere: 39.7%).

FIG. 10 depicts the specific lysis of tumor cells in an in vitro assay with A-172 cell line performed according to descriptions in example 7 in a ratio of effector cells to target cells of 5:1. Antisense oligonucleotide with Sequence Id. No. 30 was under test at a concentration of 5 microMol enhancing cell lysis. In contrast procarbacine reduced LAK induced cell lysis at a concentration of 3 nmol/ml. But in this case the cytolytic effect of Seq. Id. No. 30 (5 microM) was reduced in combination with procarbacine (3 nmol/ml). (specific cell lysis of control: 8.31%, procarbacine 6.1%, Seq. Id. No. 14: 16.4%, Seq. Id. No. 30 in combination with procarbacine: 5.7%).

### DETAILED DESCRIPTION OF THE INVENTION

References of literature, patents or publications of patent applications mentioned in the description are incorporated by reference.

The method of the present invention is applicable to any mammal. Examples of mammal to which the method may be usefully applied include laboratory animals, including rodents such as mice, rats and guinea pigs; farm animals such as cows, sheep, pigs and oats; pet animals such as dogs and cats; and primates such as monkeys, apes and humans. The invention is most preferably applied in human clinical situations, particularly where the patient is undergoing immunosuppressive therapy after organ or tissue transplantation; or any other form of surgery where suppression of the immune system of the patient is indicated. However, other mammals may also benefit from the practice of the invention. These other high value animals such as horses and fur animals such as mink.

In one embodiment of this invention the at least one stimulator of the function of the immune cells and/or the immune system and at least one substance inhibiting the cell proliferation and/or inducing cell death is a mixture of these at least two components pure or in a pharmaceutical acceptable carrier also herein referred to as combination.

In another embodiment of this invention the at least one stimulator of the function of the immune cells and/or the immune system and at least one substance inhibiting the cell proliferation and/or inducing cell death are separate in one pharmaceutical composition. Each of these parts being pure or in a pharmaceutical acceptable carrier. The at least two parts of the pharmaceutical composition have the same or different pharmaceutical acceptable carriers. To these separate parts of a pharmaceutical composition is also referred to herein as combination.

Immune cells are lymphoid cells, such as T cells, B cells, NK cells (natural killer cells), NK T cells (natural killer T cells), granulocytes, such as neutrophils, eosinophils, basophils, and mononuclear cells such as monocytes, macrophages, dendritic cells and mast cells.

An immunostimulator according to this invention is any substance inducing the function of immune cells and/or the immune system to enhanced abilities directly or indirectly reducing or inhibiting the tumor cell growth and/or inducing cell death of unwanted neoplasms in a pharmaceutical acceptable carrier.

Apparatus and/or methods inducing the function of the immune cells and/or the immune system as described above are also within the scope of this invention.

In one embodiment the immunostimulator is selected from the group of chemokines, including but not limited to lymphotactin, interleukin 1, interleukin 2, interleukin 6, interleukin 12, interferon gamma, and/or immune cell attracting substances.

In yet another embodiment the immunostimulator is selected from the group of viruses and/or parts of viruses, including retroviruses, adenoviruses, papillomaviruses, Epstein-Barr-viruses and viruses that are non-pathogenic including Newcastle-Disease virus, Cow-pox-virus

In another embodiment the immunostimulator is selected from the group of autologous, heterologous MHC-Molecules, molecules involved in antigen processing, molecules involved in antigen presentation, molecules involved in mediating immune cell effects, molecules involved in mediating immune cell cytotoxic effects, molecules involved in antigen transportation, co-stimulatory molecules, peptides enhancing recognition by immune cells and/or cytotoxic effects of immune cells.

In yet another embodiment the immunostimulators are peptides enhancing the recognition of unwanted neoplasms by immune cells and/or cytotoxic effects of immune cells containing one or more mutations and/or amino acid substitutions of the ras proteins, the p53 protein, the EGF-receptor protein, fusion peptides and/or fusion proteins, the retinoblastoma protein, proteins coded by oncogenes and/or protooncogenes and/or proteins coded by anti-oncogenes and/or tumor suppressor genes.

In yet another embodiment the immunostimulators are peptides enhancing the recognition of unwanted neoplasms by immune cells and/or cytotoxic effects of immune cells containing one or more mutations and/or amino acid substitutions caused by gene rearrangements and/or gene translocations.

In yet another embodiment the immunostimulators are peptides enhancing the recognition of unwanted neoplasm by immune cells and/or cytotoxic effects of immune cells derived from proteins differing in the target cell by one or more amino acids from the proteins expressed by other cells in the same organism.

In yet another preferred embodiment the immunostimulators are peptides enhancing the recognition of unwanted neoplasm by immune cells and/or cytotoxic effects of immune cells derived from viral antigens and/or coded by viral nucleic acids.

In yet another embodiment the immunostimulators are peptides derived from proteins expressed in a diseased organ but not in the nervous system, muscle, hematopoetic system or other organs essential for survival. Diseased organs are e.g. prostate, ovary, breast, melanine producing cells and the like.

In yet another embodiment the immunostimulator is a peptide containing one or more amino acids differing between a protein in the target cell from the other cells within an organism, tumor cell extracts, tumor cell lysates and/or adjuvants.

In yet another embodiment the immunostimulator is fusion cell of dendritic and tumor cells or is dendritic cells. These fusion cells are hybridoma cells derived from a mixture of dendritic cells and tumor cells. Dendritic cells are generated e.g. by treatment of PBMC with GM-CSF and IL-4 or a mixture of GM-CSF, IL-4 and IFN-γ, or FLT-3 ligand. Fusion of dendritic cells with tumor cells can be achieved e.g. using PEG (polyethylene glycol) or electrofusion (Hayashi, T., et al. 2002, Parkhust, M. R. 2003, Phan, V. 2003).

In yet another preferred embodiment the immunostimulator is an antagonist of factors negatively influencing the function of the immune system. These factors are e.g. TGF-beta (transorming growth factor beta), VEGF (vascular endothelial growth factor), PGE₂ (prostaglandin E₂), IL 10 (interleukin 10).

In yet another embodiment the immunostimulator is a vaccine.

Vaccines according to this invention comprise but are not limited to substance in a pharmaceutical acceptable carrier selected from the group of whole (irradiated) tumor cells, ASI (active specific immunization) with e.g. Newcastle Disease Virus (NDV) modified tumor cell vaccine (Schneider, T. et al. 2001), tumor cell lysates.

In one preferred embodiment the vaccines are peptides, peptides combined with cytokines (e.g. IL-2, IL-12, GM-CSF) or peptides combined with adjuvants (e.g. incomplete Freund's adjuvant, QS21).

In yet another embodiment of vaccination recombinant virus constructs that encode carcinoma antigen(s) are part of e.g. adenovirus, vaccinia, fowlpox and/or avipox.

In yet another embodiment the vaccine is naked DNA encoding carcinoma antigen(s).

In yet another embodiment the vaccines are dendritic cells, dendritic cells loaded with peptides derived from carcinoma antigens, dendritic cells transfected with recombinant viruses or RNA, DNA and/or cDNA encoding different tumor antigens, dendritic cells pulsed with tumor lysates and/or dendritic cells fused with whole tumor cells. For further vaccines see also Jager, E. et al. 2003.

In a preferred embodiment of this invention the immunostimulator is an antagonist of factors negatively influencing the function of the immune system.

An “antagonist” as used herein is any substance inhibiting the production of e.g. a cytokine and/or the effect of cytokines. Examples for cytokines negatively influencing the immune systems are e.g. TGF-beta, VEGF, IL-10, PGE-E₂. The inhibition in one embodiment works by binding the cytokine to a binding protein, to a receptor or to a part of this receptor, by binding the cytokine with an antibody, a low molecular substance inhibiting the cytokine or its production, or by inhibiting the signal pathway of said cytokine, e.g. by inhibiting the receptors of these cytokines or any other link downstream in the activation cascade of cytokines.

More details are given for the preferred embodiment of TGF-beta antagonists, which can be transferred to the cytokines described above as well.

Antagonist of the immune system as used herein is any substance or method inhibiting the activity of the immune system.

“Low molecular substances” or “small molecules” herein comprise substances with a molecular weight of less than about 10 kDa and more than about 1 Da of organic or anorganic origin.

In a preferred embodiment the at least one immunostimulator of the pharmaceutical composition of this invention is a TGF-beta antagonist.

TGF-beta (transorming growth factor beta) in the context of this invention comprises all subclasses of TGF-beta, preferred subclasses are TGF-beta 1, TGF-beta 2 and TGF-beta 3.

In the context of this invention a TGF-beta antagonist is any substance inhibiting the function of TGF-beta in the meaning that any effect that is induced by TGF-beta is inhibited.

In preferred embodiments TGF-beta antagonists are substances inhibiting the production of TGF-beta, are substances binding TGF-beta and/or are substances in habiting the function of TGF-beta downstream its activation cascade. For more details about TGF-beta antagonists see also Wojtowicz-Praga (2003) herein incorporated by reference. Examples for TGF-beta antagonists are given in Example 7.

In one embodiment of TGF-beta antagonists inhibiting the production of TGF-beta are oligonucleotides and/or their active derivatives hybridising with an area of the messenger RNA (mRNA) of TGF-beta and/or the DNA encoding TGF-beta and by this inhibit the production of TGF-beta.

In yet another embodiment the substance inhibiting the production of TGF-beta is a peptide, a peptide of less than 100 kDa, peptides being part of TGF-beta, a protein, a protein that is not an antibody, and/or a small molecule, e.g. tranilast (N-[3,4-dimethoxycinnamoyl]-anthranilic acid) (Wilkenson, K. A. 2000).

In one embodiment the peptides being part of TGF-beta are sequences of those given in example 8. Example 8 presents the amino acid sequences of TGF-beta 1, TGF-beta2 and TGF-beta3 also published in Mittl (1996) herein incorporated by reference.

In one preferred embodiment peptides comprise the 112 amino acids starting counting from the end of the TGF-beta1, TGF-beta2 or TGF-beta 3 peptide as written in example 8. The start of those peptides is after the RXXR motif ending 113 amino acid before the end of the TGF-beta1, TGF-beta2 or TGF-beta3 peptide, in which R is the amino acid Arginin and XX represents any amino acid or is even no amino acid.

In one embodiment peptides being part of TGF-beta are parts of the sequences presented in example 8 comprising one to all amino acids of this peptide, in other embodiments preferred peptides comprise about 1-100 amino acids, about 2-50 amino acids, about 3-30 amino acids or about 5-20 amino acids of those peptides.

In yet other embodiments preferred amino acids are those presented in example 8 for TGF-beta1, TGF-beta2 and TGF-beta3 with the respective numbers 1-21.

Further preferred embodiments are parts of amino acids as described above with about 1-50 amino acids, about 1-40, about 2-30, about 3-25, about 4-18, about 5-15 or about 6-12 amino acids.

In yet other embodiments of the peptides described above at least one of the basic amino acid selected from the group of Histidin (H), Lysin (K) and/or Arginin (R) is substituted by another basic amino acid selected from this group without loosing its TGF-beta antagonizing effects.

In yet other embodiments of the peptides described above at least one of the acid amino acid selected from the group of glutaminic acid (E) and/or asparaginic acid (D) is substituted by its counterpart of this group without loosing its TGF-beta antagonizing effects.

The peptides that are part of TGF-beta wherein some amino acids are replaced conservatively compared to their sequences presented in example 8 are also referred to as analogs of TGF-beta1, TGf-beta2 and/or TGF-beta3.

In some embodiments in the analogs of TGF betel, TGF-beta2 and TGF-beta3 1 to about 30%, about 2% to about 20%, about 3% to about 15%, 4% to about 12% or about 5% to about 10% of the amino acids are replaced conservatively.

Amino acid replaced conservatively, also referred to as conservative analogs or active derivatives of peptides in the context of this invention means replacing at least one amino acid of a peptide or protein. Preferably at least one acid amino acid (glutaminic acid (E), asparaginic acid (D)) is replaced by the respective other acid amino acid, accordingly at least one basic amino acids is replaced by another basic amino acid, at least one amino acid with a polar group (—OH, —SH, —CONH₂) is replaced by another amino acid with a polar group and/or amino acids with pure carbon side chains are replaced by another amino acid with pure carbone side chain. Peptides and/or proteins conservatively replaced with amino acids are still in the scope of this invention.

In another embodiment the peptides described above are single and not in the combination with a chemotherapeutic agent. In yet another embodiment these peptides are used for preparing a pharmaceutical composition with a pharmaceutically acceptable carrier. In yet another embodiment these peptides are comprised by a pharmaceutical composition for the treatment of neoplasms and in yet another embodiment these peptides are used for a method treating neoplasms according to this invention, more preferred glioma, astrocytoma and/or glioblastoma.

In yet another embodiment TGF-beta antagonists are receptors and/or parts of it binding TGF-beta and in that way inhibiting the function of TGF-beta.

In yet another embodiment the TGF-beta antagonist is an antibody and/or parts of it binding TGF-beta and by this inhibiting the function of TGF-beta. Those antibodies are commercially available, see e.g. R & D Systems, Inc. The production of those antibodies is well known in the art. Animals such as e.g. chicken, mice, rabbits, goats, are immunized with purified human TGF-beta. IgY then is purified with e.g. affinity chromatography as described for example by Cooper, N. M. (1995). In yet other embodiments the TGF-beta antibodies are further modified e.g. biotinylated.

In a more preferred embodiment the TGF-beta antibodies are humanized antibodies. For more details about humanized antibodies see also Carrington (1998).

In yet another embodiment the TGF-beta antagonist is a protein and/or peptide binding to TGF-beta and by this inhibiting the function of TGF-beta. Preferred embodiments of these peptides are e.g. Latency-associated peptides and can inhibit all three isoforms of TGF-beta (TGF-beta 1, TGF-beta 2 and TGF-beta 3).

In another embodiment the TGF-beta inhibitor is a protein, peptide or a small molecule inhibiting the function of the TGF-beta receptor, acting extracellularly or intracellularly.

In yet other embodiments the TGF-beta antagonists comprise, proteins, peptides, antibodies and/or small molecules, which inhibit the TGF-beta activity by inhibiting any link downstream the TGF-beta cascade of activation.

In a preferred embodiment of this invention the antagonists of a peptide, cytokine and/or receptor are nucleic acids.

The terms “nucleic acid” and “oligonucleotide” refer to multiple nucleotides (i.e. molecules comprising a sugar, e.g. ribose or deoxyribose) linked to a phosphate group and to a variable organic base, which is either a substituted pyrimidine, e.g. cytosine (C), thymine (T) or uracil (U) or a substituted purine, e.g. adenine (A) or guanine (G) or a modification thereof. As used herein, the terms refer to oligoribonucleotides as well as oligodeoxyribonucleotides. The terms shall also include oligonucleosides (i.e. a oligonucleotide without the phosphate) and any other organic base containing polymer. The nucleic acids may be double-stranded or single-stranded. Double-stranded molecules may be more stable in vivo, while single-stranded molecules may have increased activity. In one embodiment the nucleotides have lengths between about 6 and about 100 nucleotides in yet another embodiment the nucleotides have lengths of about 8 to about 40 nucleotides respectively from about 12 to about 32 nucleotides.

As used here with respect to linked units of a nucleic acid, “linked” or “linkage” means two entities are bound to one another by any physicochemical means. Any linkage known to those of ordinary skill in the art, covalent or noncovalent, is embraced. Natural linkages, which are those ordinarily found in nature connecting the individual units of a nucleic acid, are most common. The individual units of a nucleic acid may be linked, however, by synthetic or modified linkages.

In one embodiment the respective ends of this linear polymeric structure can be further joined to form a circular structure. However, open linear structures are generally preferred. Within the oligonucleotides structure, the phosphate groups are commonly referred to as forming the internucleoside backbone of the oligonucleotide. The normal linkage or backbone of RNA and DNA is a 3′ to 5′ phosphodiester linkage.

In one embodiment the terms “nucleic acids”, “nucleotides”, “oligonucleotides” respectively “antisense oligonucleotides” are substances stimulating the function of the immune system and/or the immune cells and/or are antagonists of TGF-beta as described herein. In preferred embodiments they comprise DNA- or RNA-fragments coding for TGF-beta and/or its receptors, VEGF and/or its receptors, interleukin 10 (IL-10) and/or its receptors, PGE₂ and/or its receptors or are the respective antisense nucleotides and/or are ribozymes.

In still other embodiments, the nucleic, acids are not antisense nucleic acids, meaning that they do not function by binding to complementary genomic DNA or RNA species within a cell and thereby inhibiting the function of said genomic DNA or RNA species.

In one embodiment the sequences comprises the sequences as described in the Patents EP 069 53 54 and EP 1008649 as well as those of the international patent applications published under No. WO 01/68 146, WO98/33904 and WO 99/63975 herein incorporated by reference. TGF-beta antisense oligonucleotides in one preferred embodiment include at least one sequence set forth as SEQ ID NOs: 1-127.

Oligonucleotides or nucleic acids include oligonucleotides having non-naturally occurring portions with similar function. Such modified or substituted oligonucleotides are often preferred over native forms because of desirable properties such as, for example, enhanced cellular uptake, enhanced affinity for nucleic acid target (e.g. protein), altered intracellular localization and increased stability in the presence of nucleases. Modifications of the oligonucleotides as used herein comprises any chemical modifications of the sugar, the base moiety and/or the internucleoside linkage.

In one embodiment nucleic acids or oligonucleotides with a covalently modified base and/or sugar include for example nucleic acids having backbone sugars which are covalently attached to low molecular weight organic groups other than a hydroxyl group at the 3′ and/or 2′ position and other than a phosphate group at the 5′ position. Thus modified nucleic acids may include a 2′-O-alkylated ribose group. In yet another embodiment modified nucleic acids include sugars such as arabinose instead of ribose. Thus the nucleic acids may be heterogeneous in backbone composition thereby containing any possible combination of polymer units linked together such as peptide-nucleic acids (which have amino acid backbone with nucleic acid bases). In some embodiments the nucleic acids are homogeneous in backbone composition.

The substituted purines and pyrimidines of the nucleic acids include standard purines and pyrimidines such as cytosine as well as base analogs such as substituted bases (Wagner et al. 1996). Purines and pyrimidines include but are not limited to adenine, cytosine, guanine, thymine, 5-methylcytosine, 2-aminopurine, 2-amino-6-chloropurine, 2,6-diaminopurine, hypoxanthine, and other naturally and non-naturally occurring nucleobases, substituted and unsubstituted aromatic moieties.

The single nucleotides in each oligonucleotide or polynucleotide polymer may contain the same modifications, may contain combinations of these modifications, or may combine these modifications with phosphodiester linkages. Methods of rendering oligonucleotide or polynucleotide polymers nuclease resistant include, but are not limited to, covalently modifying the purine or pyrimidine bases. For example, bases may be methylated, hydroxymethylated, or otherwise substituted (e.g., glycosylated) such that the oligonucleotides or polynucleotides are rendered substantially acid and nuclease resistant.

In a preferred embodiment, at least one end-block on the oligonucleotide is a biotin, biotin analog, avidin, or avidin analog. These molecules have the ability to both block the degradation of the protected oligonucleotide or polynucleotide and provide means for high affinity attachment of the modified nucleic acids to the solid support. Avidin and biotin derivatives which can be used to prepare the reagents of this invention include streptavidin, succinylated avidin, monomeric avidin, biocytin (biotin-epsilon-N-lysine), biocytin hydrazide, amine or sulfhydryl derivatives of 2-iminobiotin and biotinyl-epsilon-aminocaproic acid hydrazide. Additional biotin derivatives, such as biotin-N-hydroxysuccinimide ester, biotinyl-epsilon-aminocaproic acid-N-hydroxysuccinimide ester, sulfosuccinimidyl 6-(biotin amido)hexanoate, N-hydroxysuccinimideiminobiotin, biotinbromoacetylhydrazide, p-diazobenzoyl biocytin and 3-(N-maleimidopropionyl)biocytin, can also be used as end-blocking groups on the polynucleotides of the present invention.

In another embodiment the ring structure of the ribose group of the nucleotides in the modified oligonucleotide or polynucleotide has an oxygen in the ring structure substituted with N—H, N—R (with R being an alkyl or aryl substituent), S and/or methylene.

In yet another embodiment the base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, in particular an aminoethylglycine backbone. The nucleobases are bound directly or indirectly to aza nitrogen atoms of the amide portion of the backbone. Representative United States patents that teach the preparation of PNA compounds include, but are not limited to, U.S. Pat. Nos. 5,539,082; 5,714,331; and 5,719,262, each of which is herein incorporated by reference. Further teaching of PNA compounds can be found in Nielsen et al. (1991).

Further modified oligonucleotide backbones include, for example, phosphorothioates, chiral phosphorothioates, phosphorodithioates, phosphorotriesters, aminoalkylphosphorotriesters, methyl- and other alky-phosphonates including 3′-alkylene phosphonates and chiral phosphonates, phosphinates, phosphoramidates, including 3′-aminophosphoramidate and aminoalkylphosphoramidates, thionophosphoramidates, thionoalkylphosphonates, thionoalkylphosphotriesters, and boranophosphates having norm 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts, and free acid forms are also included.

In embodiments at least one nucleotide of an oligonucleotide is modified as described in one of the modifications above. The modification can either cover the oligonucleotide continuously or irregularly.

In yet another embodiment at least two modifications as described above are combined within one oligonucleotide.

In another embodiment the 1 to about 12 or 1 to about 8 or 1 to about 4 or 1 to about 2 oligonucleotides and/or nucleotide linkages at the 3′ and/or 5′ end of the oligonucleotide are modified as described above.

In one embodiment the oligonucleotides of this invention are hybridizing with a target, e.g. TGF-beta or its subtypes, VEGF, IL-10, PGE₂. Comprising in the context of this invention means that one of the oligonucleotides of the sequence listing is part of the antisense oligonucleotide of the respective m-RNA. In one embodiment even the complete antisense oligonucleotide of the m-RNA of the target is an immunostimulator in the meaning of this invention. In yet another embodiment any part of the antisense m-RNA of a target negatively influencing the function of the immune system is within the scope of this invention. This means that oligonucleotides of the sequence listing that have additionally oligonucleotides of the sequence of the respective antisense m-RNA with about 1 to about 1000 nucleotides, from about 1 to about 500, from about 1 to about 100, from about 1 to about 50, from about 1 to about 20, from about 1 to about 10, from about 1 to about 5 or from about 1 to about 2 nucleotides bound to at least one of the 3′ and/or 5′ end, in a preferred embodiment on at least one of the 2′ and/or 5′ end, are still within the scope of this invention.

The nucleotide sequence of targets of factors negatively influencing the function of immune cells and/or the immune system, as well as the respective antisense sequences, are known to persons skilled in the art. In a preferred embodiment the targets are selected from the group of m-RNA of TGF-beta 1, TGF-beta 2 and/or TGF-beta 3. The sequence of the antisense m-RNA of TGF-beta-1, TGF beta-2, TGF-beta-3, interleukin 10, VEGF and PGE₂ synthase is given in example 6.

For use in the instant invention, the nucleic acids can be synthesized de novo using any of a number of procedures well known in the art. Such compounds are referred to as ‘synthetic nucleic acids.’ For example, the b-cyanoethyl phosphoramidite method (Beaucage et al. 1981); nucleoside H-phosphonate method (Garegg et al. 1986, Froehler et al. 1986, Garegg et al. 1986, Gaffney et al. 1988). These chemistries can be performed by a variety of automated oligonucleotide synthesizers available in the market.

Alternatively, nucleic acids can be produced on a large scale in plasmids, (see, e.g., Sambrook, et al. 1989) and separated into smaller pieces or administered whole. Nucleic acids can be prepared from existing nucleic acid sequences (e.g., genomic or cDNA) using known techniques, such as those employing restriction enzymes, exonucleases or endonucleases. Nucleic acids prepared in this manner are referred to as isolated nucleic acids. The term “antineoplastic nucleic acid” encompasses both synthetic and isolated antineoplastic nucleic acids.

Modified backbone nucleic acids, such as those having phosphorothioates bonds may be synthesized using automated techniques employing, for example, phosphoramidate or H-phosphonate chemistries. Aryl- and alkyl-phosphonates can be made, e.g., as described in U.S. Pat. No. 4,469,863. Alkylphosphotriesters, in which the charged oxygen moiety is alkylated as described in U.S. Pat. No. 5,023,243 and European Patent No. 092,574, can be prepared by automated solid phase synthesis using commercially available reagents. Methods for making other nucleic acid backbone modifications and substitutions have been described (Uhlmann et al. 1990, Goodchild 1990).

Phosphorothioates may be synthesized using automated techniques employing either phosphoramidate or H-phosphonate chemistries. Aryl- and alkyl-phosphonates can be made, e.g., as described in U.S. Pat. No. 4,469,863; and alkylphosphotriesters (in which the charged oxygen moiety is alkylated as described in U.S. Pat. No. 5,023,243 and European Patent No. 092,574) can be prepared by automated solid phase synthesis using commercially available reagents. Methods for making other DNA backbone modifications and substitutions have been described (Uhlmann, E. et al. 1990, Goodchild,). 1990).

Synthesis of Peptides of this Invention

One method for synthesizing proteins and or peptides of this invention is merfield synthesis. This methodology is characterised by the use of tert-butyl based temporary □-amino protection and benzyl, or substituted benzyl, groups for permanent side chain protection. There are over one hundred different substituted resins suitable for peptide synthesis generally based on polystyrene and polyethylene glycol, which is state of the art knowledge. These resins allow introduction of an amino acid through either substitution, condensation or addition reactions. The traditional resin used for Merrifield synthesis is a chloromethylphenyl substituted resin. The first amino acid is attached to the resin through substitution of the chloride by the caesium salt of the BOC-amino acid, generating an equivalent to a benzyl ester.

Deprotection of the temporary BOC group uses a 20-50% solution of trifluoroacetic acid (TFA) in dichloromethane and has to be followed by neutralisation of the resulting ammonium salt with a hindered tertiary base. Final cleavage from the resin as well as deprotection of benzyl based side chain protecting groups is achieved using strong acids, usually liquid hydrogen fluoride or trifluoromethane sulphonic acid. Such procedures require specialised apparatus and the highly acidic conditions catalyse several possible rearrangements.

Yet another method of synthesizing proteins and/or peptides according to this invention is Fmoc Polyamide Synthesis, developed by Eric Atherton and Bob Sheppard at the Laboratory of Molecular Biology in Cambridge in the late 1970's. The fundamental differences between the Fmoc polyamide strategy when compared to the Merrifield approach are that the reactions are carried out under continuous flow and that the conditions for □-amino deprotection and cleavage from the resin are far more mild. This arises from the adoption of the base labile Fmoc protecting group for □-amino protection. The side chains are generally protected with tert-butyl based groups which, in common with the linkage to the resin, can be cleaved by TFA in the presence of scavengers.

As mentioned above, a large number of resins are available. Traditionally, resins with 4-hydroxymethylphenoxy substitution is used. These allowes were esterified with the anhydride of the first amino acid. As a result of the mesomerically electron donating para oxygen atom stabilising the resultant carbocation, cleavage of the peptide from the resin occurs under more mild acid conditions, typically using trifluoroacetic acid with scavengers.

The use of continuous flow means that the reagents are passed through a reaction chamber containing the resin supported peptide. Having passed through this chamber, the reagents can be recirculated back into the chamber again or taken to a waste collection bottle. This allows the resin to be washed clean of excess reagents and unwanted reaction products, which in turn helps drive deprotection steps to completion following Le Chatelier's principle. In addition, the solution can be passed through a u.v. detector and monitored at a suitable wavelength for the Fmoc chromophore.

A typical cycle consists of:

1. Deprotection of the preceding residue with piperidine.

2. Wash to remove any remaining reagents from 1.

3. Acylation in a recirculatory mode.

4. Wash to remove excess reagents.

Automated applied Biosystems 432A peptide synthesizer might be applied.

As used herein, the term “neoplasm” means new and abnormal growth or formation of tissue and/or blood cells in the body of a organism. The unwanted neoplasms include, but are not limited to, solid tumors; blood born tumors such as leukemias, acute or chronic myelotic or lymphoblastic leukemia; tumor metastasis; benign tumors, for example hemangiomas, acoustic neuromas, neurofibromas, trachomas, and pyogenic granulomas; pre-malignant tumors; astrocytoma, comprising pilocyt. astrocytoma WHO I, astrocytoma WHO II, astrocytoma WHO III, blastoma, chordoma, craniopharyngioma, ependymoma, Ewing's tumor, germinoma, glioma, glioblastoma, hemangioblastoma, hemangioperycatioma, Hodgkins lymphoma, medulloblastoma, leukaemia, mesothelioma, neuroblastoma, non-Hodgkins lymphoma, pinealoma, retinoblastoma, sarcoma (including angiosarcoma, chondrosarcoma, endothelial sarcoma, fibrosarcoma, leiomyosarcoma, liposarcoma, lymphangioandotheliosarcoma, lyphangiosarcoma, medulloblastoma, melanoma, meningioma, myosarcoma, neurinoma, oligodendroglioma, osteogenic sarcoma, osteosarcoma), seminoma, subependymoma, Wilm's tumor, or is selected from the group of bile duct carcinoma, bladder carcinoma, brain tumor, breast carcinoma, bronchogenic carcinoma, carcinoma of the kidney, cervical carcinoma, choriocarcinoma, cystadenocarcinome, embryonal carcinoma, epithelial carcinoma, esophageal carcinoma, cervical carcinoma, colon carcinoma, colorectal carcinoma, endometrial carcinoma, gallbladder carcinoma, gastric carcinoma, head and neck carcinoma, liver carcinoma, lung carcinoma, medullary carcinoma, non-small cell bronchogenic/lung carcinoma, ovarian carcinoma, pancreas carcinoma, papillary carcinoma, papillary adenocarcinoma, prostate carcinoma, small intestine carcinoma, rectal carcinoma, renal cell carcinoma, skin carcinoma, small-cell bronchogenic/lung carcinoma, squamous cell carcinoma, sebaceous gland carcinoma, testicular carcinoma, uterine carcinoma.

Pharmaceutical compositions of this invention beside the immunostimulator comprise at least one substance inhibiting cell proliferation and/or inducing cell death.

An “antineoplastic chemotherapeutic agent” as used herein is a substance inhibiting cell proliferation and/or inducing cell death and in a preferred embodiment further inhibits the formation of metastases not by stimulating the immune cells and/or the function of the immune system as described herein. The term antineoplastic chemotherapeutic agent comprises, but is not limited to antineoplastic agents, antineoplastic supplementary potentiating agents and radioactive agents. Examples for this group are given herein.

In one embodiment antineoplastic substances are selected from the group of telozolomid, nitrosoureas, Vinca alkaloids, antagonists of purine and pyrimidines bases, cytostatic antibiotics, camphotecine derivatives, anti-estrogenes, anti-androgens and analogs of gonadotropin releasing hormon.

In a preferred embodiment the group of nitrosoureas comprises ACNU, BCNU, CCNU.

In another embodiment the antineoplastic chemotherapeutic agent is selected from the group of nitrosoureas, e.g. ACNU, BCNU and/or CCNU, cytotoxic active antibiotics, e.g. doxorubicin, pegylated liposomal doxorubicin (Caelyx®), 5-fluorodeoxyuridine, 5-fluorouracil, 5-fluorouridine, gemcitabine, procarbazine, taxol, taxotere, temozolomide, vinblastine, vincristine.

Synonyms for ACNU are 3-[(−4-Amino-2-methyl-5-pyrimidinyl)methyl]-1-(2-chloroethyl)-1-nitrosourea hydrochloride, CS-439HCl, Nidran hydrochloride, Nimustine Hydrochloride, NSC-245382.

BCNU is Bischloroethylnitrosourea, the chemical name is N,N′-bis(2-chlorethyl)-N-nitroso-urea, other names are BiCNU, carmustine.

CCNU is 1-(2-Chloroethyl)-3-cyclohexyl-1-nitrosourea. Synonyms are N-(2-chloroethyl)-N′-cyclohexyl-N-nitroso-urea, Belustine, Cee NU, Chloroethylcyclohexylnitrosourea, ICIG 1109, Lomustine, NSC 79037.

One chemical name for temozolomide is 3,4-dihydro-3-methyl-4-oxoimidazo->5,1d′1,2,3,4-tetrazin-8-carboximide. Other names for temozolomide are Temodal, Temodar, methazolastone, CCRG81045, SCH52365, NSC362856, M&B39836.

Synonyms for teniposide are 4′-Demethylepipodophyllotoxin, 9-(4,6-O-2-thenylidene-b-D-glucopyranoside), Epipodophyllotoxin, EPT, Teniposide VM-26, VM 26, 5,8,8a,9-Tetrahydro-5-(4-hydroxy-3,5-dimethoxyphenyl)-9-{[4,6-O-(2-thienylmethylene)-b-D-glucopyranosyl]oxy}furo[3′,4′:6,7]naphtho[2,3-d]-1,3-dioxol-6(5aH)-one.

In one embodiment the Vinca alkaloids comprise vincristine, vinblastine, vindesine and their active derivatives.

In one embodiment the antagonist of the purine and pyrimidine bases is selected from the group of 5-fluorouracile, 5-fluorodeoxiuridine, cytarabine and gemcitabine.

In other embodiments the cytostatic antibiotic is selected from the group of doxorubicine and liposomal PEGylated doxorubicin, the camphthotecine derivative is selected from the group of irinotecane and topotecane, the anti estrogenes are selected from the group of tamoxifen, exemestane, anastrozole and fulvestrant, the antiandrogens are selected from the group of flutamide and bicalutamide, the antprogesterons are selected from the group of mifepriston, the analogs of gonadotropin releasing hormon are selected from the group of leuprolide and gosereline.

In other embodiments the at least one immunostimulator of this invention is combined with at least one antineoplastic agent selected from the following group:

Acivicin; Aclarubicin; Acodazole Hydrochloride; Acronine; Adozelesin; Adriamycin; Aldesleukin; Altretamine; Ambomycin; Ametantrone Acetate; Aminoglutethimide; Amsacrine; Anastrozole; Anthramycin; Asparaginase; Asperlin; Azacitidine; Azetepa; Azotomycin; Batimastat; Benzodepa; Bicalutamide; Bisantrene Hydrochloride; Bisnafide Dimesylate; Bizelesin; Bleomycin Sulfate; Brequinar Sodium; Bropirimine; Busulfan; Cactinomycin; Calusterone; Caracemide; Carbetimer; Carboplatin; Carmustine; Carubicin Hydrochloride; Carzelesin; Cedefingol; Cetuximab; Chlorambucil; Cirolemycin; Cisplatin; Cladribine; Crisnatol Mesylate; Cyclophosphamide; Cytarabine; Dacarbazine; DACA (N-[2-(Dimethylamino)ethyl]acridine-4-carboxamide); Dactinomycin; Daunorubicin Hydrochloride; Daunomycin; Decitabine; Dexormaplatin; Dezaguanine; Dezaguanine Mesylate; Diaziquone; Docetaxel; Doxorubicin; Doxorubicin Hydrochloride; Droloxifene; Droloxifene Citrate; Dromostanolone Propionate; Duazomycin; Edatrexate; Eflornithine Hydrochloride; Elsamitrucin; Enloplatin; Enpromate; Epipropidine; Epirubicin Hydrochloride; Erbulozole; Erlotinib; Esorubicin Hydrochloride; Estramustine; Estramustine Phosphate Sodium; Etanidazole; Ethiodized Oil 1131; Etoposide; Etoposide Phosphate; Etoprine; Fadrozole Hydrochloride; Fazarabine; Fenretinide; Floxuridine; Fludarabine Phosphate; Fluorouracil; 5-FdUMP; Fluorocitabine; Fosquidone; Fostriecin Sodium; Gefitinib; Gemcitabine; Gemcitabine Hydrochloride; Gold Au 198; Hydroxyurea; Idarubicin Hydrochloride; Ifosfamide; Ilmofosine; Imatinib mesylate; Interferon Alfa-2a; Interferon Alfa-2b; Interferon Alfa-n1; Interferon Alfa-n3; Interferon Beta-I a; Interferon Gamma-I b; Iproplatin; Iressa; Irinotecan Hydrochloride; Lanreotide Acetate; Letrozole; Leuprolide Acetate; Liarozole Hydrochloride; Lometrexol Sodium; Lomustine; Losoxantrone Hydrochloride; Masoprocol; Maytansine; Mechlorethamine Hydrochloride; Megestrol Acetate; Melengestrol Acetate; Melphalan; Menogaril; Mercaptopurine; Methotrexate; Methotrexate Sodium; Metoprine; Meturedepa; Mitindomide; Mitocarcin; Mitocromin; Mitogillin; Mitomalcin; Mitomycin; Mitosper; Mitotane; Mitoxantrone Hydrochloride; Mycophenolic Acid; Nocodazole; Nogalamycin; Ormaplatin; Oxisuran; Paclitaxel; Pegaspargase; Peliomycin; Pentamustine; Peplomycin Sulfate; Perfosfamide; Pipobroman; Piposulfan; Piroxantrone Hydrochloride; Plicamycin; Plomestane; Porfimer Sodium; Porfiromycin; Prednimustine; Procarbazine Hydrochloride; Puromycin; Puromycin Hydrochloride; Pyrazofurin; Riboprine; Rituximab; Rogletimide; Safinol; Safingol Hydrochloride; Semustine; Simtrazene; Sparfosate Sodium; Sparsomycin; Spirogermanium Hydrochloride; Spiromustine; Spiroplatin; Streptonigrin; Streptozocin; Strontium Chloride Sr 89; Sulofenur; Talisomycin; Taxane; Taxoid; Tecogalan Sodium; Tegafur; Teloxantrone Hydrochloride; Temoporfin; Teniposide; Teroxirone; Testolactone; Thiamiprine; Thioguanine; Thiotepa; Thymitaq; Tiazofurin; Tirapazamine; Tomudex; TOP-53; Topotecan Hydrochloride; Toremifene Citrate; Trastuzumab; Trestolone Acetate; Triciribine Phosphate; Trimetrexate; Trimetrexate Glucuronate; Triptorelin; Tubulozole Hydrochloride; Uracil Mustard; Uredepa; Vapreotide; Verteporfin; Vinblastine; Vinblastine Sulfate; Vincristine; Vincristine Sulfate; Vindesine; Vindesine Sulfate; Vinepidine Sulfate; Vinglycinate Sulfate; Vinleurosine Sulfate; Vinorelbine Tartrate; Vinrosidine Sulfate; Vinzolidine Sulfate; Vorozole; Zeniplatin; Zinostatin; Zorubicin Hydrochloride; 2-Chlorodeoxyadenosine; 2′-Deoxformycin; 9-aminocamptothecin; raltitrexed; N-propargyl-5,8-dideazafolic acid; 2-chloro-2′-arabino-fluoro-2′-deoxyadenosine; 2-chloro-2′-deoxyadenosine; anisomycin; trichostatin A; hPRL-G129R; CEP-751; linomide.

Other anti-neoplastic agents include:

20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives (e.g., 10-hydroxy-camptothecin); canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexifosfamide; deicrazoxane; dexverapamil; diaziquone; didemnin B; didox; diethylnorspermine; dihydro-5-azacytidine; dihydrotaxol, 9-; dioxamycin; diphenyl spiromustine; discodermolide; docosanol; dolasetron; doxifluridine; droloxifene; dronabinol; duocarmycin SA; ebselen; ecomustine; edelfosine; edrecolomab; eflornithine; elemene; emitefur; epirubicin; epothilones including desoxyepothilones (A, R.dbd.H; B, R.dbd.Me); epithilones; epristeride; estramustine analogue; estrogen agonists; estrogen antagonists; etanidazole; etoposide; etoposide 4′-phosphate (etopofos); exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; irinotecan; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mithracin; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticarcinoma agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; podophyllotoxin; porfimer sodium; porfiromycin; propyl bis-acridone; prostaglandin 32; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thalidomide; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene dichloride; topotecan; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; zinostatin stimalamer.

Antineoplastic Supplementary Potentiating Agents:

Tricyclic anti-depressant drugs (e.g., imipramine, desipramine, amitryptyline, clomipramine, trimipramine, doxepin, nortriptyline, protriptyline, amoxapine and maprotiline); non-tricyclic anti-depressant drugs (e.g., sertraline, trazodone and citalopram); Ca.sup.++ antagonists (e.g., verapamil, nifedipine, nitrendipine and caroverine); Calmodulin inhibitors (e.g., prenylamine, trifluoroperazine and clomipramine); Amphotericin B; Triparanol analogues (e.g., tamoxifen); antiarrhythmic drugs (e.g., quinidine); antihypertensive drugs (e.g., reserpine); Thiol depleters (e.g., buthionine and sulfoximine) and Multiple Drug Resistance reducing agents such as Cremaphor EL. The compounds of the invention also can be administered with cytokines such as granulocyte colony stimulating factor.

Antiproliferative agent: Piritrexim Isethionate.

Radioactive agents:

Fibrinogen I 125; Fludeoxyglucose F 18; Fluorodopa F 18; Insulin I 125; Insulin I 131; Iobenguane I 123; Iodipamide Sodium I 131; Iodoantipyrine I 131; Iodocholesterol I 131; Iodohippurate Sodium I 123; Iodohippurate Sodium I 125; Iodohippurate Sodium I 131; Iodopyracet I 125; Iodopyracet I 131; Iofetamine Hydrochloride I 123; Iomethin I 125; Iomethin I 131; Iothalamate Sodium I 125 Iothalamate Sodium I 131; Iotyrosine I 131; Liothyronine I 125; Liothyronine I 131; Merisoprol Acetate Hg 197; Merisoprol Acetate Hg 203; Merisoprol Hg 197; Selenomethionine Se 75; Technetium Tc 99m Antimony Trisulfide Colloid; Technetium Tc 99m Bicisate; Technetium Tc 99m Disofenin; Technetium Tc 99m Etidronate; Technetium Tc 99m Exametazime; Technetium Tc 99m Furifosmin; Technetium Tc 99m Gluceptate; Technetium Tc 99m Lidofenin; Technetium Tc 99m Mebrofenin; Technetium Tc 99m Medronate; Technetium Tc 99m Medronate Disodium; Technetium Tc 99m Mertiatide; Technetium Tc 99m Oxidronate; Technetium Tc 99m Pentetate; Technetium Tc 99m Pentetate Calcium Trisodium; Technetium Tc 99m Sestamibi; Technetium Tc 99m Siboroxime; Technetium Tc 99m Succimer; Technetium Tc 99m Sulfur Colloid; Technetium Tc 99m Teboroxime; Technetium Tc 99m Tetrofosmin; Technetium Tc 99m Tiatide; Thyroxine I 125; Thyroxine I 131; Tolpovidone I 131; Triolein I 125; Triolein I 131.

The term antineoplastic chemotherapeutic also agents includes nucleic acid molecules for the inhibition of angiogenesis and inductors of the aggregation of tubulin.

Active derivatives of the antineoplastic chemotherapeutic agents as well as prodrugs are also part of this invention.

Since a common but tolerable side effect of antineoplastic agents is nausea and vomiting it is obvious to someone skilled in the art that these effects can be avelliated by administering an anti-emetic in conjunction with the antineoplastic agent inducing nausea and/or vomiting. E.g. Ondansetron may be given p.o. in a dose of about 8 mg about 30 minutes before the nausea/vomiting inducing antineoplastic agent is administered. Of course other anti-emtics such as Hasaldol, Benadryl, and Ativan may also be used as needed.

The antineoplastic chemotherapeutic agent of this invention are commercially available. For the synthesis of e.g. temozolomid see for example Stevens et al. (1984) or Wang et. al (1994).

Radiation is applied in dosages of about 1 Gy to about 100 Gy, more preferred from about 20 to about 80 Gy and most preferred, e.g. for the treatment of astrocytomas, glioblastomas and gliomas from about 40 to about 60 Gy.

The dosage in preferred embodiments is fractionated which means that, from about 0.1 to about 10 Gy or from about 1 Gy to about 5 Gy or from about 1 Gy to about 2 Gy are applied in one session which is repeated several times during about 1 to about 20 weeks, about 2 to about 10 weeks or 4 to about 8 weeks. The antagonist and/or the substance inhibiting cell proliferation and/or inducing cell death of this invention can be administered before, after or together with the radiation. One cycle of radiation therapy as well as several cycles of radiation are possible, dependent of the reduction of tumor size.

The radiation usually is performed with ⁶⁰Co. Radiation with neutrons, protons, negative pi-mesones or neutrone capture are applicable as well.

It is clear to someone skilled in the art that the dosage is further dependant on the size of the tumor, the build of the patient and the kind of radiation applied. In special embodiments the dosage is about 2 to about 100 fold higher or lower as described above also dependant from the number of fractions the dosage is applied with.

In one embodiment the combination of at least one immunostimulator and at least one antineoplastic agent is useful in the treatment of unwanted neoplasms such as but not limited solid tumors; blood born tumors such as leukemias, acute or chronic myelotic or lymphoblastic leukemia; tumor metastasis; benign tumors, for example hemangiomas, acoustic neuromas, neurofibromas, trachomas, and pyogenic granulomas; pre-malignant tumors; astrocytoma, blastoma, chordoma, craniopharyngioma, ependymoma, Ewing's tumor, germinoma, glioma, glioblastoma, hemangioblastoma, hemangioperycatioma, Hodgkins lymphoma, medulloblastoma, leukaemia, mesothelioma, neuroblastoma, non-Hodgkins lymphoma, pinealoma, retinoblastoma, sarcoma (including angiosarcoma, chondrosarcoma, endothelial sarcoma, fibrosarcoma, leiomyosarcoma, liposarcoma, lymphangioandotheliosarcoma, lyphangiosarcoma, medulloblastoma, melanoma, meningioma, myosarcoma, neurinoma, oligodendroglioma, osteogenic sarcoma, osteosarcoma), seminoma, subependymoma, Wilm's tumor, or is selected from the group of bile duct carcinoma, bladder carcinoma, brain tumor, breast carcinoma, bronchogenic carcinoma, carcinoma of the kidney, cervical carcinoma, choriocarcinoma, cystadenocarcinome, embryonal carcinoma, epithelial carcinoma, esophageal carcinoma, cervical carcinoma, colon carcinoma, colorectal carcinoma, endometrial carcinoma, gallbladder carcinoma, gastric carcinoma, head and neck carcinoma, liver carcinoma, lung carcinoma, medullary carcinoma, non-small cell bronchogenic/lung carcinoma, ovarian carcinoma, pancreas carcinoma, papillary carcinoma, papillary adenocarcinoma, prostate carcinoma, small intestine carcinoma, rectal carcinoma, renal cell carcinoma, skin carcinoma, small-cell bronchogenic/lung carcinoma, squamous cell carcinoma, sebaceous gland carcinoma, testicular carcinoma, uterine carcinoma.

In another embodiment the composition of at least one agonist and at least one antineoplastic agent may be used in combination with other procedures for the treatment of diseases. For example, a tumor may be treated conventionally with surgery and/or radiation and then the composition of an immunostimulator and antineoplastic chemotherapeutic agent according to this invention may be subsequently administered to the patient to extend the dormancy of micrometastases and to stabilize respectively reduce any residual unwanted neoplasm.

In a preferred embodiment a combination of at least one antineoplastic agent and at least one antagonist is administered to a site likely to harbor a metastatic lesion (that may or may not be clinically discernible at the time). A sustained release formulation implanted specifically at the site (or the tissue) where the metastatic lesion is likely to be would be suitable in these latter instances.

The embodiments of the combination of at least one stimulator of the immune cells and/or the immune system and at least one substance inhibiting cell proliferation, and/or inducing cell death is delivered in effective amounts. In general, the term “effective amount” of an antagonist and/or antineoplastic agent refers to the amount necessary or sufficient to realize a desired biologic effect. Specifically, the effective amount is that amount that reduces the rate or inhibits altogether formation of neoplasms. For instance, when the subject bears a tumor, an effective amount is that amount which decreases or eliminates the unwanted neoplasm. Additionally, an effective amount may be that amount which prevents an increase or causes a decrease in new unwanted neoplasms.

The effective amount varies depending upon whether the combination is used in single or multiple dosages. Dosages given in this writing are for adults. It is quite clear to someone skilled in the art that these dosages have to be adapted if the human being is a child, a person stressed by a further illness or other circumstances.

The effective dosage is dependent also on the method and means of delivery, which can be localized or systemic. For example, in some applications, as in the treatment of skin carcinoma or ophthalmic carcinoma the combination is preferably delivered in a topical or ophthalmic carrier.

In one embodiment subject doses of the compounds described herein typically range from about 0.1 μg to about 10 mg per administration, which depending on the application could be given hourly, daily, weekly, or monthly and any other amount of time therebetween. In yet another embodiment the doses range from about 10 μg to about 5 mg per administration or from about 100 μg to about 1 mg, with 1-10 administrations being spaced hours, days or weeks apart. In some embodiments, however, doses may be used in a range even 2 to 100 fold higher or lower than the typical doses described above.

In one embodiment of this invention the at least one immunostimulator of a pharmaceutical composition according to this invention is an antagonist, more preferred an antagonist of TGF-beta and most preferred an antisense oligonucleotide of TGF-beta which is administered in a dose range from about 1 μg/kg/day to about 100 mg/kg/day or from about 10 μg/kg/day to about 10 mg/kg/day or from about 100 μg/kg/day to about 1 mg/kg/day.

In a further preferred embodiment of the pharmaceutical composition described herein the at least one immunostimulator, more preferred the TGF-beta antagonist, most preferred the TGF-beta antisense oligonucleotide is administered with a catheter directly into the unwanted neoplasm. The concentrations of these antisense oligonucleotides are from about 0.1 μM/L to about 1 M/L, more preferred from about 1 μM/L to about 500 μM/L and even more preferred from about 10 to about 200 μM/L or from about 50 μM/L to about 150 μM/L in a sterile aqueous solution. In yet another preferred embodiment this solution is administered with a flow of about 0.1 μL/min to about 50 μL/min or about 2 μL/min to about 12 μL/min or about 3 μL/min to about 10 μL/min into the neoplasm.

In yet another embodiment the at least one antineoplastic chemotherapeutic agent is selected from the group of nitrosourea, more preferred BCNU, CCNU and/or ACNU in combination with at least one immunostimulator and/or radiation is administered in dose range from about 1 mg/m² to about 1000 mg/m², more preferred in a dose of about 50 mg/m² to about 500 mg/m² and most preferred in a single doses of about 150 mg/m² to 200 mg/m² intravenously every 6 weeks. It may be given as a single dose or divided into daily injections such as about 75 mg/m² to about 100 mg/m² on two successive days.

In yet another embodiment in the treatment of neoplasms the antineoplastic chemotherapeutic agent is gemcitabine and is administered with at least one immunostimulator and/or radiation at a dosage of about 10 mg/m² to about 10 g/m², more preferred from about 100 mg to about 5 g/m² and most preferred from about 500 mg/m² to about 2000 mg/m².

In another embodiment the dosage of gemcitabine is administered within about 10 min to about 120 min, more preferred from about 15 min to about 60 min and most preferred from about 20 min to about 40 min. In yet another embodiment this single dose is administered repeatedly within about 4 to about 10 days, respectively about 5 to about 8 days and most preferred within about 7 days. About 1 to about 8, more preferred about 2 to about 6 most preferred about 3 to about 4 single doses are administered. After this a therapy free interval of about 2 to about 60 days, more preferred about 5 to about 30 days and most preferred from about 10 to about 20 days is applied. Several repetitions of these cycles are possible.

In yet another embodiment at least one antineoplastic chemotherapeutic agent is temozolomide and is administered with a total dose of about 500 to about 1200 mg/m², over a period from about 2 to about 28 consecutive days, more preferable over a period of from about 4 to about 7 consecutive days, and most preferably over a period of about 5 consecutive days. Thus if the total dose is to be about 1000 mg/m² administered over a period of about 5 days, the daily dose for this period is about 200 mg/m²/day. Temozolomide must be administered more than once per day. Preferably dosing regimes would be twice per day, three times per day or four times per day. After a period of about 28 to about 42 days, or about 28 to about 35 days, or more preferably 28 days, from the first day of temozolomide administration, another administration cycle may be started.

In yet another embodiment the temozolomide may be administered for a much longer period at reduced dosage. For example, the temozolomide could be administered more than once daily for up to six weeks at a daily dosage of about 50 mg/m²/day to about 150 mg/m² preferably at about 75 mg/m²/day. More preferred these daily doses are split about evenly into two or more doses to be administered two or more times per day.

In yet another embodiment vinblastin is administered at a dosage of about 0.1 mg/m² to about 50 mg/m² more preferred in a dose of about 1 mg/m² to about 10 mg/m² and even more preferred at about 4 mg/m² to about 8 mg/m².

In a further embodiment vincristin is administered at a dose of about 0.1 mg/m² to 10 mg/m² more preferred in a dose of about 0.5 mg/m² to about 5 mg/m² and more preferred at about 0.8 mg/m² to about 2 mg/m² about once a week whereas the neurotoxicity is the dosage limiting factor. Most commonly solution of vincristin sulfate from about 0.1 mg/mL to about 10 mg/mL are administered with single doses of about 0.1 mg/m² to about 50 mg/m² more preferred in a dose of about 0.5 mg/m² to about 10 mg/m² and even more preferred from about 1 mg/m² to about 5.0 mg/m².

In one embodiment a pharmaceutical composition for the treatment of glioma, glioblastoma and/or anaplastic astrocytoma comprises a combination of at least one immunostimulator, more preferred an antagonist of TGF-beta, even more preferred an antisense oligonucleotide of TGF-beta and most preferred, an antisense oligonucleotide identified in the sequence listing under Seq. Id. No. 1-127 and even more preferred the sequences with Seq. Id. No. 22-48 and at least one substance inhibiting cell proliferation and/or inducing cell death preferably selected from the group of temozolomide, ACNU, BCNU, CCNU, vinblastine, vincristine, vindesine and their active derivatives, 5-fluorouracile, 5-fluorodeoxiuridine, cytarabine, gemicitabine liposomal pegylated doxorubicine, procarbazine and vincristin.

In another embodiment the antineoplastic chemotherapeutic agents procarbazine, CCNU and vincristin are together with the immunostimulator, more preferred an antagonist, even more preferred an antisense oligonucleotide of TGF-beta and most preferred, an antisense oligonucleotide identified in the sequence listing under Seq. Id. No. 1-127 and even more preferred the sequence with Seq. Id. No. 22-48 are the components of a pharmaceutical composition. The dosage in this embodiment is about 40 mg/m² to about 80 mg/m² of procarbazine p.o. (days about 8 to about 21), about 80 to about 120 mg/m² CCNU, p.o. (about day 1), vincristin from about 1.2 mg/m² to about 1.8 mg/m² p.o. (day 1) with a maximum of about 2 mg/m² i.v. on about day 8, and about day 29. The immunostimulator is given before, with or after the administration of these three substances.

In another embodiment this cycle is repeated after about 6 to about 8 weeks once or several times.

In a further preferred embodiment the at least one immunostimulator more preferred an antagonist, even more preferred an antisense oligonucleotide of TGF-beta and most preferred, an antisense oligonucleotide identified in the sequence listing under Seq. Id. No. 1-127 and even more preferred the sequences with Seq. Id. No. 22-48 and telozolomide are the parts of a pharmaceutical composition. In this case the dosage of temozolomide for the treatment of unwanted neoplasms more preferred glioma, glioblastoma and/or anaplastic astrocytoma is from about 120 to about 180 mg/m², p.o. on day 1 to 5 of a cycle. In a more preferred embodiment the immunostimulator is administered from about 1 μg/kg/day to about 50 mg/kg/day. The cycle is repeated after about 3 to 5 weeks.

In a more preferred embodiment of the above mentioned embodiments for the treatment of neoploasms such as glioma, glioblastoma and/or anaplastic astrocytoma the immunostimulator is an antagonist of TGF-beta yet more preferred an antisense oligonucleotide identified in the sequence listing under Seq. Id. No. 1-127 and most preferred the oligonucleotides identified with the Seq. Id. No. 22 to 48.

In a further preferred embodiment for the treatment of glioma radiation is further administered according to standard schedules as described above. In one embodiment the radiation is applied together with the administration of the combination as described above. In other embodiments the radiation is applied before or after the administration of the pharmaceutical compositions according to this invention.

In one embodiment of pharmaceutical compositions for the treatment of neoplasms, more preferred pancreatic neoplasms at least one substance inhibiting cell proliferation and/or inducing cell death is selected from the group of cisplatin, carboplatin, cyclophosphamid, docetaxel, PEG-liposomal doxorubicin, etoposid, folinicc acid, 5-fluorouracil, mitoxantrone, paclitaxel, topotecan and/or treosulfan.

In more preferred embodiments for the treatment of neoplasms the antineoplastic chemotherapeutic agents paclitaxel or carboplatin are the at least one part of a pharmaceutical composition according to this invention. Paclitaxel from about 100 mg/m² to about 200 mg/m² more preferred about 175 mg/m² or carboplatin administered i.v. on day 1 of a cycle. This cycle is repeated after about 20 to about 30 days.

In yet another embodiment for the treatment of neoplasms such as pancreatic neoplasms the at least one antineoplastic chemotherapeutic agent of a pharmaceutical composition according to this invention is gemcitabine. Gemcitabine is administered in dosages of about 800 mg/m² to about 1200 mg/m², more preferred about 1000 mg/m² iv. Within about 10 min to about 60 min, more preferred within about 12 min to about 20 min. This application is repeated for about 5 to about 10 days.

In yet other embodiments paclitaxel together with carboplatin, docetaxel together with carboplatin, carboplatin together with cyclophosphamid, cisplatin together with treosulfan, etoposid, mitoxantron together with folin acid and 5-fluorouracil, topotect, or PEG-liposomal doxorubicin are the at least at least one antineoplastic chemotherapeutic agent of a pharmaceutical composition according to this invention for the treatment of pancreatic neoplasms.

In a more preferred embodiment of the above mentioned embodiments for the treatment of pancreatic neoplasm the antagonist is an antagonist of TGF-beta yet more preferred an antisense oligonucleotide identified in the sequence listing under Seq. Id. No. 1-127 and even more preferred the sequence with Seq. Id. No. 22-48.

In another embodiment further to the administration of these pharmaceutical compositions, radiotherapy is applied according to standard schedules as described above.

### Non-Small Cell Lung Carcinoma (NSCLC)

In one embodiment of a pharmaceutical composition for the treatment of non small cell lung carcinoma (NSCLC) the at least one antineoplastic chemotherapeutic agent is selected from the group of cisplatin, etoposid, carboplatin, mitomycin, paclitaxel, gemcitabine and vinorelbine.

In yet another embodiment for the treatment of NSCLC further radiation is applied according to schedules as described above.

In a further preferred embodiment cisplatin together with etoposid are the at least one antineoplastic chemotherapeutic agent being administered for the treatment of neoplasms such as NSCLC. In a more preferred embodiment cisplatin is administered with a dosage of about 40 mg/m² to about 80 mg/m² more preferred about 60 mg/m² is infused on about day 1 and etoposid with a dosage of about 80 mg/m² to about 150 mg/m² is infused within about 30 min to about 200 min on days 1 to 3 of a cycle. In a further preferred embodiment additionally radiation of the lung takes place with about 1 Gy to about 2 Gy, about 1 to about 2 times per day with a complete dosage of about 30 Gy to about 60 Gy within one cycle. The radiotherapy is before, in parallel or after the administration of the pharmaceutical composition according to this invention. In another preferred embodiment one cycle of this therapy comprises about 15 to about 30 days, more preferred about 22 days. About 1 to about 10 cycles are applied.

In yet another embodiment for the treatment of NSCLC the at least one substance inhibiting the cell growth cisplatin (dosage of about 20 mg/m² to about 40 mg/m²) is infused during about 1 h on about days 1, 8, 29, 36 of one cycle or cisplatin (dosages of about 4 mg/m² to about 8 mg/m²) is infused each day of a cycle. In a further preferred embodiment radiators is applied with a dosage of about 2 Gy and concomitant boost of about 0.5 Gy each day with a concomitant boost of about 0.3 to about 0.8 Gy per day and a maximum total dosage within one cycle of about 40 Gy to about 80 Gy, more preferred from about 50 Gy to about 70 Gy. The cycle has a length of about 25 to about 50 days, more preferred from about 30 to about 40 days, most preferred from about 32 to about 38 days.

In a more preferred embodiment of the above mentioned embodiments for the treatment of NSCLC the immunostimulator is an antagonist of TGF-beta yet more preferred an antisense oligonucleotide identified in the sequence listing under Seq. Id. No. 1-127 and even more preferred the sequences with Seq. Id. No. 1 to 21 are administered according to schedules as described above.

In another embodiment of this invention for the treatment of neoplasms, more preferred gastrointestinal neoplasms such as neoplasms of colon, rectum, stomach, small intestine, liver and/or oesophagus the at least one antineoplastic chemotherapeutic agent is selected from the group of capecitabin, cisplatin, epirubicin, 5-fluorouracil, metotrexate, folin acid, irinotecan, mitomycin C, oxaliplatin and vinorelbine.

In yet another embodiment for the treatment of neoplasms such as oesophageal neoplasms 5-fluorouracil and cisplatin are the two substances inhibiting cell proliferation and/or inducing cell death of the pharmaceutical composition of this invention. 5-fluorouracil with a dosage from about 800 mg/m² to about 1200 mg/m² is infused continuously, more preferred from day 1 to about day 5 of one cycle. Additionally cisplatin in a dosage from about 60 mg/m² to about 90 mg/m² is administered i.v., preferred on about day 1 of this cycle.

In even more preferred embodiments of the above mentioned embodiments for the treatment of gastrointestinal neoplasms the antagonist is an antagonist of TGF-beta yet more preferred an antisense oligonucleotide identified in the sequence listing under Seq. Id. No. 1-127 and more preferred the sequences with Seq. Id. No. 1 to 21.

In a further preferred embodiment for the treatment of neoplasms such as gastrointestinal neoplasms radiation is additionally applied with a total dosage of about 40 Gy to about 60 Gy within one cycle. Even more preferred this dosage is fractioned into about 5 times about 1 Gy to about 2 Gy per week. The cycle is repeated after about 20 to about 40 days, after about 25 to about 35 days or after about 30 days.

Further preferred embodiments are pharmaceutical compositions according to this invention for the treatment of neoplasms such as melanomas, wherein the at least one substance inhibiting cell proliferation and/or inducing cell death is selected from the group of ACNU, BCNU, CCNU, cisplatin, dacarbazine, DTIC, fotemustin, interferon alpha, interleukin-2, interferon-alpha-2-a, temozolomide, vinblastin.

In even more preferred embodiments of the above mentioned embodiments for the treatment of melanoma the immunostimulator is an antagonist of TGF-beta yet more preferred an TGF-beta antisense oligonucleotide identified in the sequence listing under Seq. Id. No. 1-127 and even more preferred the sequence with Seq. Id. No. 1 to 78.

Further preferred embodiments are pharmaceutical compositions according to this invention for the treatment of neoplasms such as prostate cancer. In a preferred embodiment the at least one substance inhibiting cell proliferation and/or inducing cell death is selected from the group of docetaxel, estramustinephosphate and mitoxantrone.

In even more preferred embodiments of the above mentioned embodiments for the treatment of neoplasms such as prostate cancer the antagonist is an antagonist of TGF-beta yet more preferred an antisense oligonucleotide identified in the sequence listing under Seq. Id. No. 1-127 and even more preferred the sequences with Seq. Id. No. 1-21.

In yet other embodiments schedules for administering the at least one substance inhibiting cell proliferation and/or inducing cell in specific indications can be taken from the state of the art literature e.g. Preiβ 2002 herein incorporated by reference.

The pharmaceutical composition of an antagonist of this invention is delivered solely or in mixtures with the at least one substance inhibiting cell proliferation and/or inducing cell death. A mixture may consist of several antineoplastic agents in addition to immunostimulatory, more preferred antagonists of factors negatively influencing the immune system, more preferred TGF-beta antagonists even more preferred TGF beta antisense oligonucleotides. These at least two substances herein is also referred to as compounds.

In one embodiment the at least two compounds are mixed and pure or in a pharmaceutically acceptable carrier. In yet another embodiment the at least two compounds of the pharmaceutical composition are separate and pure or are separate and in a pharmaceutically acceptable carrier. In one embodiment the at least two components are in the same pharmaceutically acceptable carrier, in yet another embodiment the at least two components are in different pharmaceutically acceptable carriers.

“Administering” the pharmaceutical compositions of the present invention may be accomplished by any means known to a person skilled in the art. Routes of administration include but are not limited to oral, intranasal, intratracheal, ocular, pulmonal, vaginal, rectal, parenteral (e.g. intramuscular, intradermal, intravenous, intratumoral or subcutaneous or direct injection), topical, transdermal.

In one embodiment of a pharmaceutical composition for the treatment of unwanted neoplasms, the combination of at least one substance inhibiting cell proliferation and/or inducing cell death and the at least one immunostimulator are delivered by means of a biodegradable, polymeric implant or implanted catheters.

The term “pharmaceutical composition” implicates that the liquids or substances of this composition are pure and/or combined with pharmaceutically acceptable carriers.

The term “pharmaceutical acceptable carrier” means one or more compatible solid or liquid filler, diluents or encapsulating substances which are suitable for administration to a human or other manual. The term “carrier” denotes an organic or inorganic ingredient, natural or synthetic, with which the active ingredient is combined to facilitate the application. The components of the pharmaceutical compositions also are capable of being comingled with the compounds of the present invention, and with each other, in a manner such that there is no interaction which would substantially impair the desired pharmaceutical efficacy.

Such carriers enable the compounds of the invention to be formulated as tablets, coated tablets, effervescent tablets, granules, lozenge, powders, pills, dragees, (micro)capsules, liquids, gels, syrups, slurries, suspensions, emulsions and the like, for oral ingestion by a subject to be treated.

The pharmaceutical compositions may also include granules, powders, tablets, coated tablets, (micro)capsules, suppositories, syrups, emulsions, suspensions, creams, drops, coated onto microscopic gold particles or preparations with protracted release of active compounds, in whose preparation excipients and additives and/or auxiliaries such as disintegrants, binders, coating agents, swelling agents, lubricants, flavorings, sweeteners or solubilizers are customarily used as described above.

For a brief review of present methods for drug delivery, see Langer (1990), which is incorporated herein by reference.

For oral administration, the compounds (i.e., at least one immunostimulator and at least one substance inhibiting cell proliferation and/or inducing cell death) are delivered alone without any pharmaceutical carriers or formulated readily by combining the compound(s) with pharmaceutical acceptable carriers.

In one embodiment pharmaceutical preparations for oral use are obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP).

In yet another embodiment disintegrating agents are added, such as the cross-linked polyvinyl pyrrolidone, agar, or alginic acid or a salt thereof such as sodium alginate. Optionally the oral formulations may also be formulated in saline or buffers for neutralizing internal acid conditions.

In yet another embodiment dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures.

In yet another embodiment dyestuffs or pigments are added to the tablets or dragee coatings for identification or to characterize different combinations of active compound doses.

In another embodiment pharmaceutical preparations which can be used orally “vegicaps” include push-fit capsules made of gelatin, as well as soft, sealed capsules made of gelatin and a plasticizer, such as glycerol or sorbitol. In one embodiment the push-fit capsules contains the active ingredient in a mixture with filler such as lactose, binders such as starches, and/or lubricants such as talc or magnesium stearate and, optionally, stabilizers. In another embodiment of the soft capsules, the active compounds are dissolved or suspended in suitable liquids, such as fatty oils, liquid paraffin, or liquid polyethylene glycols. In addition, stabilizers may be added.

In yet another embodiment microspheres formulated for oral administration are used, wellknown to someone skilled in the art.

The formulations for oral administration are in dosages suitable for such administration.

In yet another embodiment for buccal administration, the compositions may take the form of tablets or lozenges formulated in conventional manner.

In yet another embodiment for the administration by inhalation, the compounds for use according to the present invention may be conveniently delivered in the form of an aerosol spray, from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

Suitable pharmaceutical carriers are, for example, aqueous or saline solutions for inhalation, microencapsulated, encochleated, contained in liposomes, nebulized, aerosols.

In yet another embodiment the pharmaceutical acceptable carriers of the compounds for parenteral, intrathecal, intraventricular or intratumoral administration include sterile aqueous solutions which may also contain buffers, diluents and other suitable additives such as, but not limited to, penetration enhancers, carrier compounds and other pharmaceutical acceptable carriers or excipients.

In yet another embodiment for the systemic delivery of the compounds they are in pharmaceutical carriers for parenteral administration by injection (e.g., by bolus injection or continuous infusion). Formulations for injection may be presented in unit dosage form, e.g., in ampoules or in multi-dose containers, with an added preservative. The pharmaceutical compositions take such forms as suspensions, solutions or emulsions in oily or aqueous vehicles, and contain formulatory agents such as suspending, stabilizing and/or dispersing agents.

In one embodiment pharmaceutical carriers for parenteral administration include aqueous solutions of the active compounds in water-soluble form.

In yet another embodiment a suspension of the compounds is prepared as appropriate oily injection suspension. Suitable lipophilic solvents or vehicles include fatty oils such as sesame oil, or synthetic fatty acid esters, such as ethyl oleate or triglycerides, or liposomes. Aqueous injection suspensions comprise substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

In yet another embodiment the active compounds may be in powder form for constitution with a suitable vehicle, e.g., sterile pyrogen-free water, before use or dried onto a sharp object to be scratched into the skin.

In yet another embodiment the compounds are formulated in rectal or vaginal compositions such as suppositories or retention enemas or tablets, e.g., containing conventional suppository bases such as cocoa butter or other glycerides.

In yet another embodiment the compounds are formulated as a depot preparation. In one embodiment such long acting formulations are formulated with suitable polymeric or hydrophobic materials (for example as an emulsion in an acceptable oil) or ion exchange resins, or as sparingly soluble derivatives, for example as a sparingly soluble salt.

In other embodiments delivery systems include time-release, delayed release or sustained release delivery systems. Such systems can avoid repeated administrations of the compounds, increasing convenience to the subject and the physician. Many types of release delivery systems are available and known to those of ordinary skill in the art.

In one embodiment the delivery systems include polymer base systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109.

In another embodiment the delivery systems include non-polymer systems that are e.g. lipids including sterols such as cholesterol, cholesterol esters and fatty acids or neutral fats such as mono-, di- and triglycerides; hydrogel release systems; sylastic systems; peptide based systems; wax coatings; compressed tablets using conventional binders and excipients; partially fused implants; and the like.

Specific examples include, but are not limited to: (a) erosional systems in which an agent of the invention is contained in a form within a matrix such as those described in U.S. Pat. Nos. 4,452,775, 4,675,189, and 5,736,152, and (b) diffusional systems in which an active component permeates at a controlled rate from a polymer such as described in U.S. Pat. Nos. 3,854,480, 5,133,974 and 5,407,686. In addition, pump-based hardware delivery systems can be used, some of which are adapted for implantation.

In still other embodiments, the antagonist and antineoplastic agent are formulated with GELFOAM®, a commercial product consisting of modified collagen fibers that degrade slowly.

In one embodiment the pharmaceutical compositions also comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

In one embodiment the immunostimulators and substances inhibiting cell proliferation and/or inducing cell death are administered neat or in the form of a pharmaceutical acceptable salt. The salts have to be pharmaceutical acceptable, but non-pharmaceutical acceptable salts may conveniently be used to prepare pharmaceutical acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium or calcium salts of the carboxylic acid group.

In one embodiment suitable buffering agents include but are not limited to: acetic acid and a salt (1-2% w/v); citric acid and a salt (1-3% w/v); boric acid and a salt (0.5-2.5% w/v); and phosphoric acid and a salt (0.8-2% w/v).

Suitable preservatives include benzalkonium chloride (0.003-0.03% w/v); chlorobutanol (0.3-0.9% w/v); parabens (0.01-0.25% w/v) and thimerosal (0.004-0.02% w/v).

In one embodiment the pharmaceutically acceptable carrier for topical administration for the at least two compounds of a pharmaceutical composition according to this invention include transdermal patches, ointments, lotions, creams, gels, drops, suppositories, sprays, liquids and powders. Conventional pharmaceutical carriers, aqueous, powder or oily bases, thickeners and the like may be necessary or desirable. In yet another embodiment coated condoms, gloves and the like are useful.

In yet another embodiment the pharmaceutical compositions also include penetration enhancers in order to enhance the alimentary delivery. Penetration enhancers may be classified as belonging to one of five broad categories, i.e., fatty acids, bile salts, chelating agents, surfactants and non-surfactants (Lee et al. 1991, Muranishi 1990). One or more penetration enhancers from one or more of these broad categories may be included.

Various fatty acids and their derivatives which act as penetration enhancers include, for example, oleic acid, lauric acid, capric acid, myristic acid, palmitic acid, stearic acid, linoleic acid, linolenic acid, dicaprate, tricaprate, recinleate, monoolein (a.k.a. 1-monooleoyl-rac-glycerol), dilaurin, caprylic acid, arichidonic acid, glyceryl 1-monocaprate, 1-dodecylazacycloheptan-2-one, acylcarnitines, acylcholines, mono- and di-glycerides and physiologically acceptable salts thereof (i.e., oleate, laurate, caprate, myristate, palmitate, stearate, linoleate, etc.) (Lee et al. 1991, Muranishi 1990, El-Hariri et al. 1992). Examples of some presently preferred fatty acids are sodium caprate and sodium laurate, used singly or in combination at concentrations of 0.5 to 5%.

The physiological roles of bile include the facilitation of dispersion and absorption of lipids and fat-soluble vitamins (Brunton 1996). Various natural bile salts, and their synthetic derivatives, act as penetration enhancers. Thus, the term “bile salt” includes any of the naturally occurring components of bile as well as any of their synthetic derivatives. A presently preferred bile salt is chenodeoxycholic acid (CDCA) (Sigma Chemical Company, St. Louis, Mo.), generally used at concentrations of 0.5 to 2%.

Complex formulations comprising one or more penetration enhancers may be used. For example, bile salts may be used in combination with fatty acids to make complex formulations. Preferred combinations include CDCA combined with sodium caprate or sodium laurate (generally 0.5 to 5%).

In one embodiment additionally chelating agents are used that include, but are not limited to, disodium ethylenediaminetetraacetate (EDTA), citric acid, salicylates (e.g., sodium salicylate, 5-methoxysalicylate and homovanillate), N-acyl derivatives of collagen, laureth-9 and N-amino acyl derivatives of beta-diketones (enamines) (Lee et al. 1991; Muranishi 1990; Buur et al. 1990). Chelating agents have the added advantage of also serving as DNase inhibitors.

In yet another embodiment additionally surfactants are used. Surfactants include, for example, sodium lauryl sulfate, polyoxyethylene-9-lauryl ether and polyoxyethylene-20-cetyl ether (Lee et al. 1991); and perfluorochemical emulsions, such as FC-43 (Takahashi et al. 1988).

Non-surfactants include, for example, unsaturated cyclic ureas, 1-alkyl- and 1-alkenylazacyclo-alkanone derivatives (Lee et al. 1991); and non-steroidal anti-inflammatory agents such as diclofenac sodium, indomethacin and phenylbutazone (Yamashita et al. 1987).

In one embodiment the pharmaceutical compositions of the present invention additionally contain other adjunct components conventionally found in pharmaceutical compositions, at their art-established usage levels. Thus, for example, the compositions may contain additional compatible pharmaceutically active materials such as, e.g., antipruritics, astringents, local anesthetics or anti-inflammatory agents, or may contain additional materials useful in physically formulating various dosage forms of the composition of present invention, such as dyes, flavoring agents, preservatives, antioxidants, opacifiers, thickening agents and stabilizers. However, such materials, when added, should not unduly interfere with the biological activities of the components of the compositions of the invention.

### EXAMPLES

Clinical studies represented herein were primarily designed as safety studies and were approved by the local ethic committees and performed in accordance with the current international declaration of Helsinki for human experimentation and GCP and had to sign a written informed consent prior to recruitment.

The treatment with the antineoplastic agent followed routine schedules if nothing else is mentioned. Before the treatment with an TGF-beta antagonist, the antisense oligonucleotide of TGF-beta, with Seq. No. 30 the patients were selected according to the following criteria.

Patients had high grade gliom, either anaplastic astrocytome, WHO grade III, or glioblastoma, WHO grade IV, refractory to or recurrent after standard therapy (surgery, radiotherapy and different therapies with antineoplastic substances). Patients had not received antineoplastic agents within 10 days prior to the administration of the antagonist. Patients were between 18 and 75 years old. Karnofsky performance status (KPS) was at least 70%. Patients with clinically significant acute infections, cardiovascular abnormalities or poorly controlled seizures and pregnant and lactating females were excluded.

Surgical planning was based on computer tomography or magnetic resonance images. The perforated part of the catheter was placed in the solid, enhancing area of the tumor. Ventricles, cysts, resection cavities from prior surgical interventions, blood vessels and eloquent brain areas had to be avoided by the catheter trajectory. The catheter was introduced through a standard burr hole into the center of the largest tumor lesion. The distal end of the catheter was passed several centimetres under the galea through the skin and filled with saline. TGF-beta 2 specific antisense oligonucleotide with Seq. Id. No. 30 was administered intratumorally as a continuous high-flow microperfusion using an external pump system, Graseby 3200 (Smith Medical, London, GBM). The application system was removed after the end of the infusion. For safety assessment patients were followed up for 28 days. Post-study MRI and survival data until death were collected by the investigators.

### 1. Example

47 years old male who was diagnosed with a histologically grade III anaplastic astrocytoma received a combination therapy of several antineoplastic agents and TGF-beta 2 specific antisense oligonucleotide with Seq. Id. No. 30. The antineoplastic agents administered were ACNU together with tenoposide, temozolomide, and PEGylated liposomal doxorubicin (Caelyx®). ACNU was administered partly parallel with tenoposide with 90 mg/m² ACNU on the first day of each cycle and 60 mg/m² of tenoposide on days 1-3 of each cycle. Each cycle comprised 42 days, 4 of these cycles were realized. About 2 years later the patient was treated with 3 cycles of temozolomide. Each cycle of 28 days started with the administration of temozolomide 75 mg/m² from day 1-5. About 8 months after this treatment PEGylated liposomal doxorubicin (Caelyx®) was administered in 5 cycles of 42 days, with 20 mg/m² on day 4 and day 14 of the cycle, followed by a week with 160 mg tamoxifen administration in the morning and in the evening.

The therapy with these antineoplastic agents according to standard schedules was finally without success and therefore the patient was included into the study with TGF-beta 2 specific antisense oligonucleotide with Seq. Id. No. 30 showing surprising success. At the startpoint of this study the magnetic resonance imaging showed three tumors in the left frontal lobe and an additional tumor in the right hemisphere and an overall oedema. After the chemotherapy with the above mentioned antineoplastic agents one cycle of TGF-beta 2 specific antisense oligonucleotide with Seq. Id. No. 30 (10 μM in steril pyrogen free isotonic 0.9% NaCl solution, 4 μL/min, total of 1.42 mg in 4 days) was applied intratumorally by an implanted catheter into the largest nodule. Six months after start of TGF-beta 2 specific antisense oligonucleotide with Seq. Id. No. 30 a clear reduction of the largest tumor lesion could be diagnosed. Although not individually targeted by the catheter, the three smaller tumors also disappeared completely. Additionally, the oedema had decreased. 17 months after the first application of TGF-beta 2 specific antisense oligonucleotide with Seq. Id. No. 30 the largest tumor was hardly measurable anymore. Four months later a complete response was assessed by 3 independent specialists. These findings were accompanied by clinical improvement. The patient died due to a myocardial infarction without signs of tumor recurrence and had experienced an overall survival of 195 weeks after first recurrence and 208 weeks after diagnosis of anaplastic astrocytoma.

### 2. Example

Male patient 45 years old was diagnosed with anaplastic astrocytoma (WHO grade III). The diagnosis was followed by surgery and radiotherapy. 3 times 200 mg/m² Temozolomide was administered according to a standard schedule during two months. Again this therapy was without success. Therefore the patient was included into the study with TGF-beta 2 specific antisense oligonucleotide with Seq. Id. No. 30. Two cycles of this oligonucleotide with a concentration of 80 μM and a flow of 8 μl/min was administered for each 4 days through a catheter placed inside the tumor tissue. Afterwards the patient received ten additional cycles within four months. Following the last cycle of the oligonucleotide, approximately 10 months after the first oligonucleotide treatment, the patient received seven cycles of liposomal doxorubicin (Caelyx®).

A planned 8^{th} cycle could not be started, as the chemotherapy had to be discontinued due to cardiotoxicity (ventricular tachycardia). From that time the patient did not receive any anti-tumor therapy or corticosteroids. The last magnetic resonance image was taken 19.4 months after the start of oligonucleotide treatment. These images were evaluated and showed in the central reading a significant partial response (83% tumor reduction) and an overall survival time which was not so far reported in literature.

This is a further prove that surprisingly the coadministration of radiotherapy, antineoplastic agents and antagonists clearly show synergistic effects in the treatment of tumors, such as e.g. glioma, glioblastoma and/or astrocytoma.

### 3. Example

Comparison of survival data of patients treated with antineoplastic agents in combination with antagonists of factors negatively influencing the immune system (here: an antisense oligonucleotide of TGF-beta with the sequence Id. No. 30) to literature data for treatment with antineoplastic alone. Survival time is given from start of first chemotherapy after tumor recurrence. Median overall survival time of all patients treated with antineoplastic agents and TGF-beta 2 specific antisense oligonucleotide with Seq. Id. No. 30 (anaplastic astrocytom: 8 patients, glioblastoma, 23 patients) are compared to the most current literature data (Theodosopoulos, P. V. et al. 2001).

TABLE 1
Demographic data and patients' characteristics

| | | Age | | KPS³ at | Tumor size⁴ | Previous therapy⁶ | Seq. Id. No. 30 | | | |
|---|---|---|---|---|---|---|---|---|---|---|
| Patient | Histology¹ | (years) | Sex² | baseline | volume (cm³) | (after 1st recurrence) | (study group)⁷ | (cycles) | Steroid⁸ | |
| 01 | AA | 41 | M | 70 | 204.0 | TMZ | 1/1; 2/1 | 2x | 40 | mg MP |
| 02 | AA | 46 | M | 90 | 216.0 | Surg + TMZ, CaeTam | 1/1 | 1x | — | |
| 03 | GBM | 61 | M | 70 | 73.5 | CaeTam + Surg | 1/1 | 1x | 8 | mg MP |
| 04 | AA | 46 | M | 80 | 11.5⁵ | Surg + TMZ | 1/2 | 1x | 12 | mg DEX |
| 06 | GBM | 51 | F | 70 | 76.8 | Surg + TMZ | 1/2 | 1x | — | |
| 07 | GBM | 53 | M | 70 | 54.9 | Surg + TMZ, Surg + | 1/2 | 1x | 24 | mg DEX |
| | | | | | | Ixotene | | | | |
| 08 | GBM | 56 | M | 70 | 101.0 | TMZ | 1/2 | 1x | 2 | mg DEX |
| 09 | GBM | 63 | F | 70 | 67.7⁵ | — | 1/3 | 1x | 3 | mg DEX |
| 10 | GBM | 30 | M | 90 | 160.7 | Surg + TMZ, CaeTam | 1/3 | 1x | 12-8 | mg DEX |
| 11 | GBM | 43 | M | 70 | 16.7⁵ | TMZ | 1/3 | 1x | 2 | mg DEX |
| 12 | GBM | 58 | M | 70 | n/e | Surg + TMZ | 1/3 | 1x | 6 | mg DEX |

| | | | | | | | | | |
|---|---|---|---|---|---|---|---|---|---|
| 13 | GBM | 58 | F | 90 | 47.1⁵ | — | 1/4 | 1x | — |
| 14 | AA | 54 | M | 80 | 7.2 | Surg + TMZ | 1/4 | 1x | — |
| 15 | GBM | 42 | M | 70 | 33.6 | Surg + TMZ, 2x Surg | 1/4 | 1x | — |
| 16 | GBM | 45 | M | 90 | 27.0 | — | 1/4 | 1x | — |
| 17 | AA | 44 | M | 100 | 6.2⁵ | Surg | 1/5; 2/2 | 2x | — |
| 18 | GBM | 46 | M | 70 | n/e | TMZ + Surg | 1/5 | 1x | — |
| 19 | GBM | 41 | F | 70 | 58.8 | Surg + TMZ | 1/5 | 1x | — |

| | | | |
|---|---|---|---|
| Median | 46 | 70 | 56.85 |

¹AA, anaplastic astrocytoma; GBM, glioblastoma multiforme
²F, female; M, male
³KPS, Karnofsky performance status
⁴Tumor size at baseline; n/e = not evaluable
⁵Multiple lesions, the total volume of all lesions is presented
⁶TMZ, temozolomide; CaeTam, Caelyx ® + Tamoxifen; Surg, surgery
⁷For details see Table 1
⁸DEX, dexamethasone; MP, methylprednisolone

Summary of patients' characteristics from the study. Patients 01, 13 and 16 received each two cycles of pegylated liposomal doxorubicin (Caelyx®), patient 14 two cycles of PCV (procarbazine, lomustine (CCNU), vincristine): after TGF-beta 2 specific antisense oligonucleotide with Seq. Id. No. 30 treatment, all other patients had no anti-tumor therapy after oligonucleotide treatment. Patient 17 received 10 additional oligonucleotide cycles. After the last cycle of the oligonucleotide the patient received 7 cycles of pegylated liposomal doxorubicin.

Reduction of tumor volumes of patients 04 and 17 was more than 80%. Tumor volume was assessed by measurement of the largest cross-sectional diameter of the enhancing lesion in the first layer and the largest cross-sectional diameter perpendicular to the first in the same plane and layer. For the third dimension, the largest cross-sectional diameter of all further planes perpendicular to the first one was determined.

Compared to literature data for the treatment with antineoplastic agents alone the survival data show clearly enhanced survival of patients treated with one or more antineoplastic agents (e.g. temozolomide and/or procarbazine) before the administration of TGF-beta 2 specific antisense oligonucleotide with Seq. Id. No. 30.

The data are calculated after start of chemotherapy. According to this approach the median overall survival in our study was 147 weeks for AA and 42.4 weeks for GBM. The data reveal longer median overall survival times if applying the oligonucleotide following chemotherapy (mainly temozolomide) than the comparable published data for temozolomide alone, for which the most recent and accurate survival data are available: about 147 weeks versus 42 (Theodosopoulos, P. V. et al. 2001) weeks for anaplastic astrocytoma, and 45 weeks versus about 32 weeks (Theodosopoulos, P. V. et al. 2001; Yung, W. K. et al. 2000; Yung, W. K. 2000; Brandes, A. A. et al. 2001) for GBM, respectively.

These results surprisingly show that there is a clear survival advantage of patients treated with a combination of the antagonist, TGF-beta 2 specific antisense oligonucleotide with Seq. Id. No. 30 and at least one further antineoplastic agent (e.g. temozolomide) in patients suffering from neoplasm, e.g. AA (mean overall survival of 146.6 weeks versus 90 weeks for all anaplastic astrocytoma patients).

### Example 4

Temozolomide may be administered orally in capsule form wherein it is admixed with conventional pharmaceutical carriers. An example for a temozolomide capsule formulation is:

| | |
|---|---|
| Ingredient | mg/capsule |

| | | | | |
|---|---|---|---|---|
| Temozolomide | 5 | 20 | 100 | 250 |
| Anhydrous Lactose NF | 132.8 | 182.2 | 175.7 | 154.3 |
| Sodium Starch Glycolate NF | 7.5 | 11.0 | 15.0 | 22.5 |
| Colloidal Silicon Diozide NF | 0.2 | 0.2 | 0.3 | 0.7 |
| Tartaric Acid NF | 1.5 | 2.2 | 3.0 | 9.0 |
| Steric Acid NF | 3.0 | 4.4 | 6.0 | 13.5 |
| Capsule Size* | 3 | 2 | 1 | 0 |

*white opaque, preservative free, two piece hard gelatin capsules

The TGF-beta2 antisense oligonucleotide identified by the Seq. No. 30 is solved under sterile conditions in a sterile, pyrogene-free 0.9% NaCl solution and is ready for administration into a catheter surgically implanted with its perforated end placed in the tumor. The catheter is connected with a commercially available port system into which the AP12009 solution is administered.

### Example 5

Antisense m-RNA for the human transforming growth
factor TGF-beta 1:
CTGCAGCCTTGACCTCCCAGGATCAAGTGATCCTCCCACCTTAGCCTCCA
GAGTAGCTGGGACCACAGGTGTACATTTTTTAAAAGTGTTTTGTAGAGAT
AGGGTCTCACTATGTTACCCAGGCTGGTCTCAAATGCCTGGATTCAAGTA
TCCTCCCATCTCTGCCTCCCAAAAGTGCTAGGATTACAGGCGTGAGCACC
CCGCCTGGCCTGAACTACTATCTTTTATTGTCTTCTTCACTATCCCCCAC
TAAAGCAGGTTCCTGGTGGGCAGGAACTCCTCCCTTAACCTCTCTGGGCT
TGTTTCCTCAACCTTTAAAATGGGTGTTATCAGAGTCCCTGCCATCTCAG
AGTGTTGCTATGGTGACTGAATGAGTTCATTAATGTAAGGCACTTCAACA
GTGCCCAAGGTGCTCAATAAATAGATCTAACTACAGTAGTGTTCCCCACT
GGTCCCCTGTGCCTTGATGCCGGGCAAAGGAATAGTGCAGACAGGCAGGA
GGAGGCAGAGAGGGAGAGAGAGGGAGTGGGAGTGGGGGAACGTCAGGGAT
GGAGACCCCAGGCAGGCGCCCAATGACACAGAGATCCGCAGTCCTCTCTC
CATCTTTAATGGGGCCCCAGGTGGGCTTGGGGCACGGTGTCCTTAAATAC
AGCCCCCATGGGCAAGGCAGCGGGGGCGGGGCGGGGTGGGGCCGGGCCTG
CCGGGGCGGGGCGGGGCGGGGCGGGACCTCAGCTGCACTTGCAGGAGCGC
ACGATCATGTTGGACAGCTGCTCCACCTTGGGCTTGCGGCCCACGTAGTA
CACGATGGGCAGCGGCTCCAGCGCCTGCGGCACGCAGCACGGCGCCGCCG
AGGCGCCCGGGTTATGCTGGTTGTACAGGGCCAGGACCTTGCTGTACTGC
GTGTCCAGGCTCCAAATGTAGGGGCAGGGGCCCGAGGCAGAAGTTGGCAT
GGTAGCCCTTGGGCTCGTGGATCCACTTCCAGCCGAGGTCCTTGCGGAAG
TCAATGTACAGCTGCCGCACGCAGCAGTTCTTCTCCGTGGAGCTGAAGCA
ATAGTTGGTGTCCAGGGCTCGGCGGTGCCGGGAGCTTTGCAGATGCTGGG
CCCTCTCCAGCGGGGTGGCCATGAGAAGCAGGAAAGGCCGGTTCATGCCA
TGAATGGTGGCCAGGTCACCTCGGCGGCCGGTAGTGAACCCGTTGATGTC
CACTTGCAGTGTGTTATCCCTGCTGTCACAGGAGCAGTGGGCGCTAAGGC
GAAAGCCCTCAATTTCCCCTCCACGGCTCAACCACTGCCGCACAACTCCG
GTGACATCAAAAGATAACCACTCTGGCGAGTCGCTGGGTGCCAGCAGCCG
GTTGCTGAGGTATCGCCAGGAATTGTTGCTGTATTTCTGGTACAGCTCCA
CGTGCTGCTCCACTTTTAACTTGAGCCTCCTCAGCAGACGCAGCCTGCCC
GGGAGAGCAACACGGGTTCAGGTACCGCTTCTCGGAGCTCTGATGTGTTG
AAGAACATATATATGCTGTGTGTACTCTGCTTGAACTTGTCATAGATTTC
GTTGTGGGTTTCCACCATTAGCACGCGGGTGACCTCCTTGGCGTAGTAGT
CGGCCTCAGGCTCGGGCTCCGGTTCTGCACTCTCCCCGGCCACCCGGTCG
CGGGTGCTGTTGTACAGGGCGAGCACGGCCTCGGGCAGCGGGCCGGGCGG
CACCTCCCCCTGGCTCGGGGGGCTGGCGAGCCGCAGCTTGGACAGGATCT
GGCCGCGGATGGCCTCGATGCGCTTCCGCTTCACCAGCTCCATGTCGATA
GTCTTGCAGGTGGATAGTCCCGCGGCCGGCGGGCCAGGCGTCAGCACCAG
TAGCCACAGCAGCGGTAGCAGCAGCGGCAGCAGCCGCAGCCCGGAGGGCG
GCATGGGGGAGGCGGCGCCCCCCGGCACTGCCGAGAGCGCGAACAGGGCT
GGTGTGGTGGGGAGGCCCCGCCCCTGCAGGGGCTGGGGGTCTCCCGGCAA
AAGGTAGGAGGGCCTCGAGGGAAAGCTGAGGCTCCTCAGGGAGAAGGGCG
CAGTGGTGGAGGGGAGGCTTGGACCGGGGGTGTCTCAGTATCCCACGGAA
ATAACCTAGATGGGCGCGATCTGGTACCAGAAGGTGGGTGGTCTTGAATA
GGGGATCTGTGGCAGGTCGGAGAGAGATCCGTCTCCTGGAGGAGAAAGGG
TCTAGGATGCGCGGGGGCTCAGGAGACAGGCCGGGGATGAAGGCGGCGTG
CAGGGGGTGCGCCCGAGGTCTGGGGAAAAGTCTTTGCGGGAGGCCGGGTC
GGCGACTCCCGAGGGCTGGTCCGGAATGGGGGCGCCTGAGGGACGCCGTG
TAGGGGGCAGGGAGGGAGCAAGCGTCCCCGGCGGCAAAGGGAGGCGGTCT
GGGGTCCCCAAGTCCTGCCTCCTCGCGGGGCAGCGTCGCGCCAAGAGGTC
CCCGCGCCTCCGGCTCCCAGCGGCAACGGAAAAGTCTCAAAAGTTTTTTT
CCTCTTCTCCCGACCAGCTCGTCCCTCCTCCCGCTCCTCCTCCCCCTCCT
CCCCGCAGTGGCGGGGGCGGCGGCGGCTCGTCTCAGACTCTGGGGCCTCA
GGCTGCTCCTCGGCGACTCCTTCCTCCGCTCCGGGCCGAGGCCGGCCCCG
CGGGCGGCTCAGAGCCGGGGGGGGTGCCCCGGACGGGGCGTCCCCCCTGC
CCCCGGCCGGGGCCCTCGCTGTCTGGCTGCTCCGCGGAGGGAGGT
Antisense m-RNA of the human transforming growth
factor TGF-beta2:
TTTAAAAAAATTTGCTTCTTGTCTCTCTCACTTACAAAGTAGGTGAAATG
TAGAATAAGGCCTTCAACTTTTTTTGTGTCAGATGCCAGTTTTAACAAAC
AGAACACAAACTTCCAAAGTGTCTGAACTAGTACCGCCTTTTCAAAAATT
TTTTAACACTGATGAACCAAGGCTCTCTTATGTTTTCTTGTTACAAGCAT
CATCGTTGTCGTCGTCATCATCATTATCATCATCATTGTCATTTTGGTCT
TGCCACTTTTCCAAGAATTTTAGCTGCATTTGCAAGACTTTACAATCATA
TTAGAAAGCTGTTCAATCTTGGGTGTTTTGCCAATGTAGTAGAGAATGGT
TAGAGGTTCTAAATCTTGGGACACGCAGCAAGGAGAAGCAGATGCTTCTG
GATTTATGGTATTATATAAGCTCAGGACCCTGCTGTGCTGAGTGTCTGAA
CTCCATAAATACGGGCATGCTCCAGCACAGAAGTTGGCATTGTACCCTTT
GGGTTCGTGTATCCATTTCCACCCTAGATCCCTCTTGAAATCAATGTAAA
GTGGACGTAGGCAGCAATTATCCTGCACATTTCTAAAGCAATAGGCCGCA
TCCAAAGCACGCTTCTTCCGCCGGTTGGTCTGTTGTGACTCAAGTCTGTA
GGAGGGCAATAACATTAGCAGGAGATGTGGGGTCTTCCCACTGTTTTTTT
TCCTAGTGGACTTTATAGTTTTCTGATCACCACTGGTATATGTGGAGGTG
CCATCAATACCTGCAAATCTTGCTTCTAGTTCTTCACTTTTATTTGGGAT
GATGTAATTATTAGATGGTACAAAAGTGCAGCAGGGACAGTGTAAGCTTA
TTTTAAATCCCAGGTTCCTGTCTTTATGGTGAAGCCATTCATGAACAGCA
TCAGTTACATCGAAGGAGAGCCATTCGCCTTCTGCTCTTGTTTTCACAAC
TTTGCTGTCGATGTAGCGCTGGGTTGGAGATGTTAAATCTTTGGACTTGA
GAATCTGATATAGCTCAATCCGTTGTTCAGGCACTCTGGCTTTTGGGTTC
TGCAAACGAAAGACTCTGAACTCTGCTTTCACCAAATTGGAAGCATTCTT
CTCCATTGCTGAGACGTCAAATCGAACAATTCTGAAGTAGGGTCTGTAGA
AAGTGGGCGGGATGGCATTTTCGGAGGGGAAGAAGGGCGGCATGTCTATT
TTGTAAACTCCTTGGCGTAGTACTCTTCGTCGCTCCTCTCGCGCTCGCAG
GCGGCCGCCCTCCGGCTCGCCTTCTCCTGGAGCAAGTCCCTGGTGCTGTT
GTAGATGGAAATCACCTCCGGGGGGACTTCCTCGGGCTCAGGATAGTCTT
CTGGGGGACTGGTGAGCTTCAGCTTGCTCAGGATCTGCCCGCGGATCGCC
TCGATCCTCTTGCGCATGAACTGGTCCATATCGAGTGTGCTGCAGGTAGA
CAGGCTGAGCGCGACCGTGACCAGATGCAGGATCAGAAAAGCGCTCAGCA
CACAGTAGTGCATTTTTTAAAAAAGTGGAAAAAAAAGTTGTTTTTAAAAG
TCAGAATAAAAAAAAAGAAATCAACAATTCTCAAAGTATAGATCAAGGAG
AGTTGTTTGGTTTTTGTTGTTGTTGTTTGTTTTTGATGCGAAACTTTTGC
AAACAATCTAGTCAATGCCCAACAGAAAAACGTATCCTGCTTG
Antisense of m-RNA of the human TGF-beta 3
CAGGATGCCCCAAAAATATTTATTTATACAAAGATTTTGAGAGTAATATT
CATACTTGTCTTTATACCTCAGTCTATGCGTCTGGGGCCAAGTCACTGTG
TGGCACATGTCGAGCTTCCCCGAATGCCTCACATGTTGTCGCACCTGCTT
CCAGGAACACCAAATGAACACAGGGTCTTGGAGGGGAAGTGGGGGAAGAA
CCCATAATGCCCCAACCCTGCATGGAACCACAATCCAGAAATGTGCATCC
TGACCTGGAAGGCGTCTAACCAAGTGTCCAAGGGGAAATATGATCGAGGG
AGAGGTGAGAGGAGGGACCCAGAGGCAGACAGGAGAGGGTTGATTTCCAC
CCTTTCTTCTGCGTTAGCATATCCAAAAGGCCCAATACAGTTGATGGGCC
AGGAACTGCATGACCTGGATTTTCTCCCTGTAGTGACCCACGATGTTAAT
TGATGTAGAGGACAGTTTGCAAAAGTAATAGATTTGCCCTTAATCCCAGA
CAGTATGAGATACAATTCTGGGACTTTGTCTTCGTAACCTGTCTTTAAAA
AAAAAAAAAAATGCTTGCCTTGTATAACATAATCCAGATTCCCTAGAGCA
GATGTGGTACAGCAATGAGCAAATCCAACCTCAGATCTGAAGTGTCTTCC
AGTCTGGCCCTGACCCAGCCATTCTCTGCCCTTCCTTCTCCCTTTAGGGT
AGCCCAAATCCCATTGCCACACAACATCTCAACTTACCATCCCTTTCCTC
TATCCCCATCCCCTCTGTCTGCGTCACAGAAAGTCTGTGTGTTCTGAAGA
GTTCAGCCTTCCTCTAACCAAACCCACACTTTCTTTACCACCGTGATTCT
CAGAGCCAGCAAGAAAGAAATGTTCCAAAAGGAAACCTCCATCTCAGCCA
TTTGCCCGGAGCCGAAGGTTGTGGGCTCCAGGCCTCTCAGTGAGGTTTGT
TGCTTGTGTGTTTCCCGAGGAGCGGGCAGTCAGGCAGTGGTGGTTCTCTC
TCCCCTCTCTCTGTCGCACGTGGGGTCTCAGCTACATTTACAAGACTTCA
CCACCATGTTGGAGAGCTGCTCCACTTTGGGGGTCCTCCCAACATAGTAC
AGGATGGTCAGGGGCTCCAGGTCCTGGGGCACGCAGCAAGGCGAGGCAGA
TGCTTCAGGGTTCAGAGTGTTGTACAGTCCCAGCACCGTGCTGTGGGTTG
TGTCTGCACTGCGGAGGTATGGGCAAGGGCCTGAGCAGAAGTTGGCATAG
TAGCCCTTAGGTTCATGGACCCACTTCCAGCCCAGATCCTGTCGGAAGTC
AATGTAGAGGGGGCGCACACAGCAGTTCTCCTCCAAGTTGCGGAAGCAGT
AATTGGTGTCCAAAGCCCGCTTCTTCCTCTGACCCCCCTGGCCCGGGTTG
TCGAGCCGGTGTGGGGGAATCATCATGAGGATTAGATGAGGGTTGTGGTG
ATCCTTCTGCTTCTTGAGGCGCCCCAGATCTCCACGGCCATGGTCATCCT
CATTGTCCACGCCTTTGAATTTGATTTCCATCACCTCGTGAATGTTTTCC
AGGATATCTCCATTGGGCTGAAAGGTGTGACATGGACAGTGAATGCTGAT
TTCTAGACCTAAGTTGGACTCTCTTCTCAACAGCCACTCACGCACAGTGT
CAGTGACATCAAAGGACAGCCACTCGGCAGTGCCCCGTGTGGGCAGATTC
TTGCCACCGATATAGCGCTGTTTGGCAATGTGCTCATCTGGCCGAAGGAT
CTGGAAGAGCTCGATCCTCTGCTCATTCCGCTTAGAGCTGGGGTTGGGCA
CCCGCAAGACCCGGAATTCTGCTCGGAATAGGTTGGTTCTATTTTTCTCC
ACTGAGGACACATTGAAGCGGAAAACCTTGGAGGTAATTCCTTTAGGGCA
GACAGCCAGTTCGTTGTGCTCCGCCAGCCCCTGGATCATGTCGAATTTAT
GGATTTCTTTGGCATAGTATTCCGACTCGGTGTTTTCCTGGGTGCAGCCT
TCCTCTCCCCATGCATCTCCTCCAGCAGCTCCCGGGTGCTGTTGTAAAGG
GCCAGGACCTGATAGGGGACGTGGGTCATCACCGTTGGCTCAGGGGGGCT
GGTGAGCCTGAGCTTGCTCAAGATCTGTCCCCTAATGGCTTCCACCCTCT
TCTTCTTGATGTGGCCGAAGTCCAAGGTGGTGCAAGTGGACAGAGAGAGG
CTGACCGTGGCAAAGTTCAGCAGGGCCAGGACCACCAGAGCCCTTTGCAA
GTGCATCTTCATGTGTGAGCTGGGAAGAGAGGCCAGGGGGACGGCAAGGC
CTGGAGAGGAAGAGACCCCAGCAGACGTGCAGAAGGAGGGAGGAAAACCA
GGCGGCCTCCCCAGATCCCAAAGACTGAGGCTTGGCAAGAAGGTGCATGA
ACTCACTGCACTGCGAGAGCTTCAGGACTTCCAGGAAGCGCTGGCAACCC
TGAGGACGAAGAAGCGGACTGTGTGCCTTGTAGCGCTGGGATTCTTGTCC
ATGTGTCTAAACAGGTTTTGCTGG
Antisense of m-RNA of human Interleukin 10
TCACCCTATGGAAACAGCTTAAAAACAGGTGAAAATAATAAATATTGAAA
AAAATTATAATATTGGGCTTCTTTCTAAATCGTTCACAGAGAAGCTCAGT
AAATAAATAGAAAATGGGGGTTGAGGTATCAGAGGTAATAAATATTCTAT
AAGAGAGGTACAATAAGGTTTCTCAAGGGGCTGGGTCAGCTATCCCAGAG
CCCCAGATCCGATTTTGGAGACCTCTAATTTATGTCCTAGAGTCTATAGA
GTCGCCACCCTGATGTCTCAGTTTCGTATCTTCATTGTCATGTAGGCTTC
TATGTAGTTGATGAAGATGTCAAACTCACTCATGGCTTTGTAGATGCCTT
TCTCTTGGAGCTTATTAAAGGCATTCTTCACCTGCTCCACGGCCTTGCTC
TTGTTTTCACAGGGAAGAAATCGATGACAGCGCCGTAGCCTCAGCCTGAG
GGTCTTCAGGTTCTCCCCCAGGGAGTTCACATGCGCCTTGATGTCTGGGT
CTTGGTTCTCAGCTTGGGGCATCACCTCCTCCAGGTAAAACTGGATCATC
TCAGACAAGGCTTGGCAACCCAGGTAACCCTTAAAGTCCTCCAGCAAGGA
CTCCTTTAACAACAAGTTGTCCAGCTGATCCTTCATTTGAAAGAAAGTCT
TCACTCTGCTGAAGGCATCTCGGAGATCTCGAAGCATGTTAGGCAGGTTG
CCTGGGAAGTGGGTGCAGCTGTTCTCAGACTGGGTGCCCTGGCCTGGGCT
GGCCCTCACCCCAGTCAGGAGGACCAGGCAACAGAGCAGTGCTGAGCTGT
GCATGCCTTCTTTTGCAAGTCTGTCTTGTGGTTTGGTTTTGCAAGAGCAA
CCCCCTGATGTGTAGACCTTCACCTCTCTGTCCCCCTTTTATATTGTAAG
CTCAGGGAGGCCTCTTCATTCATTAAAAAGCCACAATCAAGGTTTCCCGG
CACAGGATTTTTTCTGCTTAGAGCTCCTCCTTCTCTAACCTCTCTAATAA
ACTTAGTTTTCAATTTTTGCATCGTAAGCAAAAATGATTGGTTGAACATG
AACTTCTGCATTACAGCTATTTTTAGGATGGGCTACCTCTCTTAGAATAA
TTTTTTAGCTTCTCAATTAAAAAAAGTTGATTTCCTGGGGAGAACAGCTG
TTCTGTCCGCAGAGGCCCTCAGCTGTGGGTTCTCATTCGCGTGTTCCTAG
GTCACAGTGACGTGGACAAATTGCCCATCCAGAATACAATGGGATGAGAA
ATAATTGG
Antisense m-RNA of human Prostaglandin E2 Synthase
tttttttttttttttttttttttttttttttttttttttttttttttttt
ttttttCCATGAGATGCCTGCCATGACAGGCGCCACAAACCTTTCCTTTA
TTGCAAACATGTCCCAGTCCCGGGAGGCTTGGGAAGAGTGGGAACCAGGG
GAACCCAGGGATGGGATTCCACTGAAAACAAACCGTCCTGCTGTCCTGTC
GAGGGCCCCCACCCACAGGATGTAGCCATGGGACAGCCACTGAGGGTCCA
GGAAGAGGGGCGGCAGAGCAGGGAGGCAGGGACAGGGAGGGGTCGCCCCA
GGGCAGTGGCAGGGCTGGAACTCGTCCCTAACATCCCTGAGCCCCAGCAG
GTGCCCTGTGTTAGAAGCGAGAGGGCTGGTGGGGGTGCGTGGACAAGGGG
CAGAATGATCCTGCCCCCAACCAGTATCGCCAGGCGCTGGCCCAGTGGCC
CCAGGCCCTGGCAGCTGGCGTCTTCCGCTGCCTCCCTCTGCTCTGCGCGG
GGACATTCAGTGCGCTGGGGAGGCCTCGGTGATGGCCCTCTCCACCCGCA
GGTACCAGGGCTGGATGTGCGTGTGCTGCATCAGGTCATCGAATGCATCC
AGCCCCTCCATCACACGCAGCACGCCATACACCGCCAAATCAGCGAGATT
CGGCTTCTGGCCCCCCATGAAGGGCCGGTCCTTGCCCAGAGCAGCCACCC
ACTTGTCAGCAGCCTCATAGAGGTCCTCGCGCACGTTGTCCTGGAGGCGG
TGCCTGCTCTTGAGTCGCTTGCTGATGAGGTACATGGCCGCTGCACCCAT
GTACTTGGCCACGGCACCCTCCACGGCTCCGAACTTGCCCTCGCGGACAA
TGTAGTCAAAGGACGCCAGAGCCTCGGTGGGCGTGCGGTACACATTGGGG
GAGATCAGGTGCACCAGCCAGTCGTCCGCCCACTGCCGCCACTTCATCTC
CTCCGTCCTGGCCTCCTTCCCACCATACACTTGCTGGGCCTCCTTCTCGT
TGAGCATGAGCCAGTACTTATTGCCGAACTCGGTCACCTCCTTGCCCTGC
TCGTTCACAGCCTTCATGGCTGGGTAGTAGGTGATGATCTCTTCCAGGGG
CTGCCCCGACACCAGGTAGGTCTTGAGGGCGCTGATGATGACAGAGGAGT
CATTTAGTTGTTGCGAGCTTTCTCCTTCCTGGGCCACCAGGATGGGCACC
TTTCTGTAGGAGGAGAACTTGATCTCAGCCCTGCGCACAGGGTTCACCTC
CACCACCTGGTAGGGCAGGGCATGGAAGTCGAGGAAGGCTCGGACCTTGC
TGCAGAAGGGACACGTCTTGTACTGGTACAGGGTCAGCTGCAGGCGGCTG
GACAGGGAGAGCTGCGCGGCTGAGCGCTCTGCGTGGAGGTCCTGGGCGCG
CAGGTGCCACCGCGCCGTGTGGTACAGCCCCAGGGCTCCCCCCAGGGCCA
GCGCCGCAGCTCCCAGCAGCCGCGGGCTCCCCTTACGAGCTGCAGCCACG
GGGCTCGGGCCGCCCGCCGCCCCCGCGAAGCCAGCCCGGCTCTGCGTGGG
TAGCAGCGGCTGGGGGCGGCCTCCCAGCCTCCAGGCCAAGGCGCACCCAC
CAGGCCACAGCGCCCGCACCACCCGCGCAGCCGGGTCCATGTTCGCTCCG
CCGGCGCCGCGGGCGGGCGCGCGAAACGAAGACGCCGAGGCACGCGCGGC
GTTTAAAGGGCCAGGACTCTGGCGCCCCGCGGGTTGGCCGGGGTGAGGGC
GACGCTAAGGGAACCCTCAGCGCTCTCGGGACTGGGCGTGTGCCCGGCGC
CCAAGTTCGAAACGCCCGCC
Antisense m-RNA of human VEGF
CAGTGTGCTGGCGGCCGCGGTGTGTCTACAGGAATCCCAGAAATAAAACT
CTCTAATCTTCCGGGCTCGGTGATTTAGCAGCAAGAAAAATAAAATGGCG
AATCCAATTCCAAGAGGGACCGTGCTGGGTCACCCGCCCGGGAATGCTTC
CGCCGGAGTCTCGCCCTCCGGACCCAAAGTGCTCTGCGCAGAGTCTCCTC
TTCCTTCATTTCAGGTTTCTGGATTAAGGACTGTTCTGTCGATGGTGATG
GTGTGGTGGCGGCAGCGTGGTTTCTGTATCGATCGTTCTGTATCAGTGTC
TTTCCTGGTGAGAGATCTGGTTCCCGAAACCCTGAGGGAGGCTCCTCCTC
CTGCCCGGCTCACCGCCTCGGCTTGTCACATCTGCAAGTACGTTCGTTTA
ACTCAAGCTGCCTCGCCTTGCAACGCGAGTCTGTGTTTTTGCAGGAACAT
TTACACGTCTGCGGATCTTGTACAAACAAATGCTTTCTCCGCTCTGAGCA
AGGCCCACAGGGATTTTCTTGTCTTGCTCTATCTTTCTTTGGTCTGCATT
CACATTTGTTGTGCTGTAGGAAGCTCATCTCTCCTATGTGCTGGCCTTGG
TGAGGTTTGATCCGCATAATCTGCATGGTGATGTTGGACTCCTCAGTGGG
CACACACTCCAGGCCCTCGTCATTGCAGCAGCCCCCGCATCGCATCAGGG
GCACACAGGATGGCTTGAAGATGTACTCGATCTCATCAGGGTACTCCTGG
AAGATGTCCACCAGGGTCTCGATTGGATGGCAGTAGCTGCGCTGATAGAC
ATCCATGAACTTCACCACTTCGTGATGATTCTGCCCTCCTCCTTCTGCCA
TGGGTGCAGCCTGGGACCACTTGGCATGGTGGAGGTAGAGCAGCAAGGCG
AGGCTCCAATGCACCCAAGACAGCAGAAAGTTCATGGTTTCGGAGGCCCG
ACCGGGGCCGGGCCGGCTCGCGCCGGGCCGCCAGCACACTG

### Example 6

### Small Molecules Inhibiting TGF-Beta

SB-431542 TβRI kinase inhibitor from GlaxoSmithKline (Callahan et al. 2002, Laping et al. 2002, Inman et al. 2002)

NPC30345 TβRI kinase inhibitor from Scios, Inc. (Dumont & Arteaga 2003)

SD-093 TβR-I kinase inhibitor (Subramanian, G. et al. 2003)

LY364947 TβRI kinase inhibitor from Lilly Inc. (Sawyer et al. 2003).

Decorin a small chondroitin-dermatan sulfate proteoglycan that binds various forms of active TGF-β (Border et al. 1992).

Proteins inhibiting TGF-beta

Endoglin a TGF-β binding 95 kDa glycoprotein (Gougos et al. 1992).

Antibodies binding TGF-beta

CAT-192 humanized TGF-beta1 mAB from Genzyme/CAT (Benigni et al. 2003).

CAT-152 humanized TGF-beta2 mAB from Genzyme/CAT (Siriwardena et al. 2002).

1D11 TGF-beta1, 2, 3 mAB from Genzyme/CAT (Ananth et al. 1999).

2G7 TGF-beta1, 2, 3 monoclonal IgG2 from Genentech., (Arteaga et al. 1993).

Antibodies against TGF-beta1/2/3 from R&D

see e.g. catalog 614 R&D systems, McKinley Place Nebr., Minneapolis, Minn. USA 55413

rabbit anti-TGF-beta2 LAP: (Schlotzer-Schrehardt, U. et al. 2001).

Soluble Receptors

sT13R11:Fc (R11/Fc hu IgG1 fusion protein) from Biogen (Muraoka et al. 2002, Rowland-Goldsmith et al. 2001)

sTβR11:Fc (Yang, Y. A. et al. 2002)

Betaglycan (recombinant soluble TβRIII) (Bandyopadhyay et al. 2002)

### Example 7

### Cell-Mediated Cytotoxicity Assay

Cell-mediated cytotoxicity was quantified by the CARE-LASS assay (Lichtenfels et al. 1994) using the NSCLC (non small cell lung carcinoma cell) line NCI-H661, the glioma cell line A-172, and the pancreatic cancer cell line Hup-T3 as target cells. NCI-H661 cells were pretreated with 5 μM TGF (transforming growth factor)-beta 1 specific antisense phosphorothioate oligodeoxynucleotide (PTO) Seq. Id. No. 14. A-172 and Hup-T3 cells were pretreated with 5 μM TGF-beta 2 specific antisense phosphorothioate oligodeoxynucleotide Seq. Id. No. 30 in medium at 5% CO₂ and 37° C. for 3 days according to the cell line suppliers' instructions. Additionally, for Hup-T3 cells 3 μg/ml Lipofectin® were used to enhance cellular uptake of the PTO. Untreated cells and cells treated with 3 μg/ml Lipofectin® were used as controls.

Lymphokine activated killer cells (LAK cells) were used as effector cells. LAK cells were generated by a 3 day pretreatment of 5×10⁶ PBMC from healthy volunteers with 10 ng/ml rh IL-2 (recombinant human interleukin 2) in 4 ml RPMI 1640 medium supplemented with 10% fetal calf serum at 5% CO₂ and 37° C. One part of the effector cells was incubated additionally with rh TGF-□ to mimic the presence of tumor cells (2000 μg/ml rh TGF-□1 for NCI-H661, 500 μg/ml rh TGF-□2 for A-172 and Hup-T3). The other part of cells was incubated without additional treatment.

Effector cells were incubated with the target cells with and without cytostatic drugs for 4 h. The supraaditive effect respectively the inhibition was calculated by subtracting the specific cell lysis, of the control from the specific cell lysis of the chemotherapeutic agent. Taking into account the sign this difference is summed up with the specific lysis of the inhibitor of TGF-beta, as depicted in the figures. In case the specific lysis of the combination of a chemotherapeutic agent with the TGF-beta inhibitor was higher than this sum, this was interpreted as supraaditive effect. In case the specific lysis of this combination was smaller than the sum this was interpreted as inhibition.

### Example 8

Presented are the amino acid sequences of TGF-beta1, TGF-beta2 and TGF-beta 3 with the international one letter abbreviation for amino acids.

RXXR: cleavage site of the mature (active) part (XX may be anything)

ASPC: the C of this motif is the C for the intermolecular cystine bridge that links the two monomers into a functional dimer

C C C: intramolecular cystein bridges (cystein knot motif)

mature protein of TGF-beta 1, 2 and 3 contains 112 amino acids from the end of this listing

TGF-beta 1
MPPSGLRLLLLLLPLLWLLVLTPGRPAAGLSTCKTIDMELVKRKRIEAIR
GQILSKLRLASPPSQGEVPPGPLPEAVLALYNSTRDRVAGESAEPEPEPE
ADYYAKEVTRVLMVETHNEIYDKFKQSTHSIYMFFNTSELREAVPEPVLL
SRAELRLLRLKLKVEQHVELYQKYSNNSWRYLSNRLLAPSDSPEWLSFDV
TGVVRQWLSRGGEIEGFRLSAHCSCDSRDNTLQVDINGFTTGRRGDLATI
HGMNRPFLLLMATPLERAQHLQSSRHRRALDTNYCFSSTEKNCCVRQLYI
DFRKDLGWKWIHEPKGYHANFCLGPCPYIWSLDTQYSKVLALYNQHNPGA
SAAPCCVPQALEPLPIVYYVGRKPKVEQLSNMIVRSCKCS

preferred amino acid sequences of TGF-beta1:

| | |
|---|---|
| 1) | ALDTNYCFSSTEKNCCVRQL |
| 2) | YIDFRKDLGWKWIHEPKGYH |
| 3) | ANFCLGPCPYIWSLDTQYSK |
| 4) | VLALYNQHNPGASAAPCCVP |
| 5) | QALEPLPIVYYVGRKPKVEQ |
| 6) | LSNMIVRSCKCS |
| 7) | TEKNCCVRQLYIDFRKDLGW |
| 8) | KWIHEPKGYHANFCLGPCPY |
| 9) | WSLDTQYSKVLALYNQHNP |
| 10) | GASAAPCCVPQALEPLPIVY |
| 11) | YVGRKPKVEQLSNMIVRSCKCS |
| 12) | QYSKVLALYNQHNPGASAAPCCVPQALEPLPIVYYVGRKP |
| 13) | QYSKVLALYNQHNPGASAAPCCVPQALEPLPIVYYVGRKP |
| | I |
| | QYSKVLALYNQHNPGASAAPCCVPQALEPLPIVYYVGRKP |

(dimer of the TGF-beta1 amino add sequence No. 12 coupled by an s-s bridge at the Cytosins of the AAPC motif)

| | |
|---|---|
| 14) | ALDTNYCFSSTEKNCCVRQLYIDFRKDLGWKWIHEPKGYHANFCLG |
| | PCPYIWSLDTQYSKVLALYNQHNPGASAAPCCVPQALEPLPIVYYV |
| | GRKPKVEQLSNMIVRSCKCS |
| 15) | ALDTNYCFSSTEKNCCVRQLYIDFRKDLGW |
| 16) | KWIHEPKGYHANFCLGPCPYIWSLDTQYSK |
| 17) | VLALYNQHNPGASAAPCCVPQALEPLPIVY |
| 18) | YVGRKPKVEQLSNMIVRSCKCS |
| 19) | CVRQLYIDFRKDLGWKWIHEPKGYHANFCL |
| 20) | GPCPYIWSLDTQYSKVLALYNQHNPGASAA |
| 21) | PCCVPQALEPLPIVYYVGRKPKVEQLSNMI |

TGF-beta 2
MHYCVLSAFLILHLVTVALSLSTCSTLDMDQFMRKRIEAIRGQILSKLKL
TSPPEDYPEPEEVPPEVISIYNSTRDLLQEKASRRAAACERERSDEEYYA
KEVYKIDMPPFFPSENAIPPTFYRPYFRIVRFDVSAMEKNASNLVKAEFR
VFRLQNPKARVPEQRIELYQILKSKDLTSPTQRYIDSKVVKTRAEGEWLS
FDVTDAVHEWLHHKDRNLGFKISLHCPCCTFVPSNNYIIPNKSEELEARF
AGIDGTSTYTSGDQKTIKSTRKKNSGKTPHLLLMLLPSYRLESQQTNRRK
RALDAAYCFRNVQDNCCLRPLYIDFKRDLGWKWIHEPKGYNANFCAGACP
YLWSSDTQHSRVLSLYNTINPEASASPCCVSQDLEPLTILYYIGKTPKIE
QLSNMIVKSCKCS

Preferred amino acid sequences of TGF-beta2

| | |
|---|---|
| 1) | ALDAAYCFRNVQDNCCLRPL |
| 2) | YIDFKRDLGWKWIHEPKGYN |
| 3) | ANFCAGACPYLWSSDTQHSR |
| 4) | VLSLYNTINPEASASPCCVS |
| 5) | QDLEPLTILYYIGKTPKIEQ |
| 6) | LSNMIVKSCKCS |
| 7) | VQDNCCLRPLYIDFKRDLGW |
| 8) | KWIHEPKGYNANFCAGACPY |
| 9) | LWSSDTQHSRVLSLYNTINP |
| 10) | EASASPCCVSQDLEPLTILY |
| 11) | YIGKTPKIEQLSNMIVKSCKCS |
| 12) | QHSRVLSLYNTINPEASASPCCVSQDLEPLTILYYIGKTPK |
| 13) | QHSRVLSLYNTINPEASASPCCVSQDLEPLTILYYIGKTPK |
| | I |
| | QHSRVLSLYNTINPEASASPCCVSQDLEPLTILYYIGKTPK |

(dimer of the TGF-beta2 amino acid sequence No. 12 coupled by an s-s bridge at the Cytosins of the ASPC motif)

| | |
|---|---|
| 14) | ALDAAYCFRNVQDNCCLRPLYIDFKRDLGWKWIHEPKGYNANFCAGA |
| | CPYLWSSDTQHSRVLSLYNTINPEASASPCCVSQDLEPLTILYYIGK |
| | TPKIEQLSNMIVKSCKCS |
| 15) | ALDAAYCFRNVQDNCCLRPLYIDFKRDLGW |
| 16) | KWIHEPKGYNANFCAGACPYLWSSDTQHSR |
| 17) | VLSLYNTINPEASASPCCVSQDLEPLTILY |
| 18) | YIGKTPKIEQLSNMIVKSCKCS |
| 19) | CLRPLYIDFKRDLGWKWIHEPKGYNANFCA |
| 20) | GACPYLWSSDTQHSRVLSLYNTINPEASAS |
| 21) | PCCVSQDLEPLTILYYIGKTPKIEQLSNMI |

TGF-beta3
MKMHLQRALVVLALLNFATVSLSLSTCTTLDFGHIKKKRVEAIRGQILSK
LRLTSPPEPWMTHVPYQVLALYNSTRELLEEMHGEREEGCTQENTESEYY
AKEIHKFDMIQGLAEHNELAVCPKGITSKVFRFNVSSVEKNRTNLFRAEF
RVLRVPNPSSKRNEQRIELFQILRPDEHIAKQRYIGGKNLPTRGTAEWLS
FDVTDTVREWLLRRESNLGLEISIHCPCHTFQPNGDILENIHEVMEIKFK
GVDNEDDHGRGDLGRLKKQKDHHNPHLILMMIPPHRLDNPGQGGQRKKRA
LDAAYCFRNVQDNCCLRPLYIDFKRDLGWKWIHEPKGYNANFCAGACPYL
WSSDTQHSRVLSLYNTINPEASASPCCVSQDLEPLTILYYIGKTPKIEQL
SNMIVKSCKCS

preferred amino acid sequences of TGF-beta3:

| | |
|---|---|
| 1) | ALDTNYCFRNLEENCCVRPL |
| 2) | YIDFRQDLGWKWVHEPKGYY |
| 3) | ANFCSGPCPYLRSADTTHST |
| 4) | VLGLYNTLNPEASASPCCVP |
| 5) | QDLEPLTILYYVGRTPKVEQ |
| 6) | LSNMVVKSCKCS |
| 7 | NLEENCCVRPLYIDFRQDLG |
| 8 | WKWVHEPKGYYANFCSGPCP |
| 9) | YLRSADTTHSTVLGLYNTLN |
| 10) | PEASASPCCVPQDLEPLTIL |
| 11) | YYVGRTPKVEQLSNMVVKSCKCS |
| 12) | THSTVLGLYNTLNPEASASPCCVPQDLEPLTILYYVGRTPK |
| 13) | THSTVLGLYNTLNPEASASPCCVPQDLEPLTILYYVGRTPK |
| | I |
| | THSTVLGLYNTLNPEASASPCCVPQDLEPLTILYYVGRTPK |

(dimer of the TGF-beta3 amino acid sequence No. 12 coupled by an s-s bridge at the cytosins of the ASPC motif)

| | |
|---|---|
| 14) | ALDAAYCFRNVQDNCCLRPLYIDFKRDLGWKWIHEPKGYNANFCAGA |
| | CPYLWSSDTQHSRVLSLYNTINPEASASPCCVSQDLEPLTILYYIGK |
| | TPKIEQLSNMIVKSCKCS |
| 15) | ALDAAYCFRNVQDNCCLRPLYIDFKRDLGW |
| 16) | KWIHEPKGYNANFCAGACPYLWSSDTQHSR |
| 17) | VLSLYNTINPEASASPCCVSQDLEPLTILY |
| 18) | YIGKTPKIEQLSNMIVKSCKCS |
| 19) | CLRPLYIDFKRDLGWKWIHEPKGYNANFCA |
| 20) | GACPYLWSSDTQHSRVLSLYNTINPEASAS |
| 21) | PCCVSQDLEPLTILYYIGKTPKIEQLSNMI |

### REFERENCES

Patents and Patent Applications

| | | |
|---|---|---|
| EP Pat. No. 069 53 54 | Schlingensiepen et al. | Jan. 09, 2002 |
| EP Pat. No. 100 86 49 | Schlingensiepen et al. | Mar. 26, 2003 |
| EP Pat. No. 009 25 74 | Tullis | Dec. 27, 2000 |
| U.S. Pat. No. 6,455,689 | Schlingensiepen et al. | Sep. 24, 2002 |
| U.S. Pat. No. 5,539,082 | Nielsen et. al. | Jul. 23, 1996 |
| U.S. Pat. No. 5,714,331 | Buchardt, deceased, et al. | Feb. 03, 1998 |
| U.S. Pat. No. 5,719,262 | Buchardt, deceased, et al. | Feb. 17, 1998 |
| U.S. Pat. No. 4,469,863 | Ts'o et al. | Sep. 04, 1984 |
| U.S. Pat. No. 5,023,243 | Tullis | Jun. 11, 1991 |
| U.S. Pat. No. 5,075,109 | Tice et al. | Dec. 24, 1991 |
| U.S. Pat. No. 4,452,775 | Kent | Jun. 05, 1984 |
| U.S. Pat. No. 4,675,189 | Kent et al. | Jun. 23, 1987 |
| U.S. Pat. No. 5,736,152 | Dunn | Apr. 07, 1998 |
| U.S. Pat. No. 3,854,480 | Zaffaroni | Dec. 17, 1974 |
| U.S. Pat. No. 5,133,974 | Paradissis et al. | Jul. 28, 1992 |
| U.S. Pat. No. 5,407,686 | Patel, et al. | Apr. 18, 1995 |
| WO 98/33 904 | Schlingensiepen et al. | published |
| | | Aug. 06, 1998 |
| WO 99/63 975 | Schlingensiepen et al. | published |
| | | Dec. 16, 1999 |
| WO 01/68 146 | Schlingensiepen et al. | published |
| | | Dec. 20, 2001 |

### OTHER REFERENCES

Alvino E. et al., J. Pharmacol. Exp. Ther. 291: 1292-1300, 1999
Ananth et al., Carcinoma Res. 59(9): 2210-6, 1999
Arteaga et al., 3 Clin Invest. 92(6): 2569-76, 1993
Bandyopadhyay et al., Carcinoma Res. 62: 4690-4695, 2002
Beaucage, S. L., Caruthers, M. H., Tet. Let. 22: 1859, 1981
Benigni et al., J. Am. Soc. Nephroi. 2003 July; 14(7):1816-24, 2003
Bernego et al. 1984
Border et al., Nature 360: 361-364, 1992
Brandes, A. A. et al., Ann Oncol 12: 255-257, 2001
Brunton, Chapter 38 In: Goodman & Gilman's, The Pharmacological Basis of Therapeutics, 9th Ed.
Buur et al., 3. Control Rel. 14: 43-51, 1990
Callahan, J. F. et al., I Med. Chem. 45: 999-1001, 2002
Carrington L., Allamby D., McLeod D., Boulon M., Incest. Opthalmol. Vis. Sci. 39: 566, 1998
Cooper, H. M. & Paterson, Y., Current protocols in Immunology 2.4.1-2.5.17, 1995
Dumont, Arteaga. Carcinoma Cell, 3: 531-536, 2003
Einstein A. B. Jr., Fass L., Fefer A., Carcinoma Res. 35(3): 492-496, 1975
El-Hariri et al., J. Pharm. Pharmacol. 44: 651-654, 1992
Froehler et al., Nucl. Acid. Res. 14: 5399-5407, 1986
Gaffney et al., Tet. Let. 29:2619-2622, 1988
Garegg et al., Tet. Let. 27:4051-4054, 1986
Gereis M., Burford-Mason A. P., Watkins S. M., Suppression of in vitro peripheral blood lymphocyte mitogenesis by cytotoxic drugs commonly used in the treatment of breast carcinoma, 1987
Giampietri A., Bonmassar E., Goldin A., J. Immunopharmacol 79 1(1), 61-86, 1978
Goodchild, J., Bioconjugate Chem. 1:165, 1990
Gougos et al., Int Immunol. 4(1): 83-92, 1992
Hardman et al., eds., McGraw-Hill, New York, N.Y.: 934-935, 1996
Hayashi, T. et al., Clin. Immunol. 104: 14-20, 2002
Inman et al., Mol. Pharmacol. 62: 65-74, 2002
Jager, E., Jager, D., Knuth, A., Int. J. Carcinoma 106: 817-820, 2003
Jantscheff, P., Spagnoli, G., Zajac, P., Rochlitz, C. F., Carcinoma Immunol Immunother 51: 367-375, 2002
Langer, Science 249: 1527-1533, 1990
Laping et al., Mol. Pharmacol. 62: 58-64, 2002
Lee et al., Critical Reviews in Therapeutic Drug Carrier Systems 8:2, 91-192, 1991
Lichtenfels et al.; Journal of Immunological Methods 172: 227-239, 1994
Lieberman, D. M., Laske, D. W., Morrison, P. F., Bankiewicz, K. S., Oldfield, E. H. J Neurosurg 82: 1021-1029, 1995
Mittl p., Priestle J. P., Cox D. A., McMaster G., Cerletti N., Grater G., The crystal structure of TGF-beta 3 and comparison to TGF-beta 2: Implications for receptor binding, Protein Science 5 1261-1271, 1996
Morrison, P. F., Laske, D. W., Bobo, H., Oldfield, E. H. & Dedrick, R. L., Am. J. Physiol. 266: R 292-305, 1994
Muranishi, Critical Reviews in Therapeutic Drug Carrier Systems, 7:1, 1-33, 1990
Muraoka et al., J. Clin. Immunol. CI 109: 1551-1559, 2002
Nardelli B., Puccetti p., Romani L., Sava G., Bonmassar E., Fioretti M. C., Carcinoma Immunol. Immunother. 17(3) 213-217, 1984
Nielsen et al., Science 254: 1497-1500, 1991
Parkhurst, M. R., DePan, C., Riley, J. P., Rosenberg, S. A., Shu, S., J. Immunol. 170, 5317-5325, 2003
Phan, V. et al., Nat Med 9: 1215-1219, 2003
Preiβ, Dornhoff, Hagmann, Schmieder, Empfehlungen zur Therapy, 11. Auflage, W. Zuckscherdt Verlag GmbH, München, Bern, Wien, New York, 2002
Rowland-Goldsmith et al., Clin. Carcinoma Res. 7: 2931-2940, 2001
Sambrook, et al., Molecular Cloning: A Laboratory Manual, Cold Spring Harbor Laboratory Press, New York, 1989
Sawyer et al., J. Med. Chem. 46(19): 3953-3956, 2003
Schlotzer-Schrehardt, U., Zenkel, M., Kuchle, M., Sakai, L. Y., Naumann, G. O., Exp. Eye Res. 73, 765-780, 2001
Schneider, T., Gerhards, R., Kirches, E. Firsching, R., J Neurooncol 53: 39-46, 2001
Siriwardena et al., Ophtalmology 109: 427-431, 2002
Stauder, G. et al. Proc. Am. Soc. Clin. Oncol. 22: 109 (abstr 436), 2003
Stevens et al., J. Med. Chem. 27: 196-201, 1984
Subramanian, G., Schwarz, R., Liu, D., Reiss, M., Roles in the pathogenesis of carcinoma and other diseases B 26, La Jolla, 2003
Takahashi et al., J. Pharm. Pharmacol., 40: 252-257, 1988
Theodosopoulos, P. V. et al., Proc Annu Meet Am Soc Clin Oncol, 20 Abstract 2059 (San Francisco, 2001)
Timmermann J. M., Czerwinski D. K., Davis T. A., Hsu F. J., Benike C., Hao, Z. M., Taidi B., Fajapaksa R., Caspar C. B., Okada C. Y., van Beckhoven A., Liles T. M., Eengleman E. G., Levy R., Blood 99(5): 1517-1528, 2002
Uhlmann, E., Peyman, A., Chem. Rev. 90: 544, 1990
de Visser, K. E., Kast, W. M., Leukemia 13: 1188-99, 1999
Wagner et al., Nature Biotechnology 14: 840-844, 1996
Wang et al., J. Chem. Commun. 1687-1688, 1994
Wilkenson, K. A., Martin, T. D., Reba S. M., Aung H., Redline R. W., Boom W. H., Toossi Z., Fulton S. A., Infect. immune. 86(11): 6505-6508, 2000
Wojtowicz-Praga, S., J. Immunother. 20, 165-77, 1997
Wojtowicz-Praga, S., Investigational New Drugs 21: 21-32, 2003
Yamashita et al., J. Pharm. Pharmacol. 39: 621-626, 1987
Yang, Y. A. et al. J. Clin. Invest. 109: 1607-1615, 2002
Yung, W. K. et al., Br. J. Carcinoma 83: 588-893, 2000
Yung, W. K., Semin Oncol 27: 27-34, 2000

### Sequences:

| | | | | |
|---|---|---|---|---|
| Seq.Id.No. | Sequence | Length | No. int. | Bez. int. |

| | | | | | |
|---|---|---|---|---|---|
| TGF-beta 1 | 1 | CGATAGTCTTGCAG | 14 | 1 | |
| | 2 | GTCGATAGTCTTGC | 14 | 2 | |
| | 3 | CTTGGACAGGATCT | 14 | 3 | |
| | 4 | CCAGGAATTGTTGC | 14 | 4 | |
| | 5 | CCTCAATTTCCCCT | 14 | 5 | |
| | 6 | GATGTCCACTTGCA | 14 | 6 | |
| | 7 | CTCCAAATGTAGGG | 14 | 7 | |
| | 8 | ACCTTGCTGTACTG | 14 | 8 | |
| | 9 | GTAGTACACGATGG | 14 | 9 | |
| | 10 | CACGTAGTACACGA | 14 | 10 | |
| | 11 | CATGTTGGACAGCT | 14 | 11 | |
| | 12 | GCACGATCATGTTG | 14 | 12 | |
| | 13 | TGTACTCTGCTTGAAC | 16 | 13 | |
| | 14 | CTGATGTGTTGAAGAACA | 18 | 14 | |
| | 15 | CTCTGATGTGTTGAAG | 16 | 15 | |
| | 16 | GGAAGTCAATGTACAG | 16 | 16 | |
| | 17 | CATGTCGATAGTCTTGCA | 18 | 17 | |
| | 18 | AGCTGAAGCAATAGTTGG | 18 | 18 | |
| | 19 | GTCATAGATTTCGTTGTG | 18 | 19 | |
| | 20 | CTCCACTTTTAACTTGAG | 18 | 20 | |
| | 21 | TGCTGTATTTCTGGTACA | 18 | 21 | |
| TGF-beta 2 | 22 | CACACAGTAGTGCA | 14 | 1 | |
| | 23 | GCACACAGTAGTGC | 14 | 2 | |
| | 24 | GCTTGCTCAGGATCTGC | 17 | 3 | |
| | 25 | TACTCTTCGTCGCT | 14 | 4 | |
| | 26 | CTTGGCGTAGTACT | 14 | 5 | |
| | 27 | GTAAACCTCCTTGG | 14 | 6 | |
| | 28 | GTCTATTTTGTAAACCTCC | 19 | 7 | |
| | 29 | GCATGTCTATTTTGTAAACC | 20 | 8 | |
| | 30 | CGGCATGTCTATTTTGTA | 18 | 9 | |
| | 31 | GGCATCAAGGTACC | 14 | 10 | |
| | 32 | CTGTAGAAAGTGGG | 14 | 11 | |
| | 33 | ACAATTCTGAAGTAGGGT | 18 | 12 | |
| | 34 | TCACCAAATTGGAAGCAT | 18 | 13 | |
| | 35 | GCTTTCACCAAATTGGAAGC | 20 | 14 | |
| | 36 | CTGGCTTTTGGGTT | 14 | 15 | |
| | 37 | TCTGATATAGCTCAATCC | 18 | 16 | |
| | 38 | TCCTAGTGGACTTTATAG | 18 | 17 | |
| | 39 | TTTTTCCTAGTGGACT | 16 | 18 | |
| | 40 | CAATTATCCTGCACATTTC | 19 | 19 | |
| | 41 | GCAATTATCCTGCACA | 16 | 20 | |
| | 42 | GCAGCAATTATCCTGC | 16 | 21 | |
| | 43 | TGGCATTGTACCCT | 14 | 22 | |
| | 44 | TGTGCTGAGTGTCT | 14 | 23 | |
| | 45 | CCTGCTGTGCTGAGTG | 16 | 24 | |
| | 46 | CTTGGGTGTTTTGC | 14 | 25 | |
| | 47 | TTTAGCTGCATTTGCAAG | 18 | 26 | |
| | 48 | GCCACTTTTCCAAG | 14 | 27 | |
| TGF-beta 3 | 49 | TCGAGCTTCCCCCA | 14 | 107 | TGF-β3-98-1 |
| | 50 | CCCCGAGCCCAAGG | 14 | 108 | TGF-β3-98-2 |
| | 51 | CCCGACGAGCCGG | 13 | 109 | TGF-β3-98-3 |
| | 52 | ACGCACCAAGGCGA | 14 | 110 | TGF-β3-98-4 |
| | 53 | CGGGTTGTCGAGCCC | 15 | 111 | TGF-β3-98-5 |
| | 54 | CGGCAGTGCCCCG | 13 | 112 | TGF-β3-98-6 |
| | 55 | CGCAATTCTGCTCG | 14 | 113 | TGF-β3-98-7 |
| | 56 | TTCGTTGTGCTCCC | 14 | 114 | TGF-β3-98-8 |
| | 57 | ATTCCGACTCGGTG | 14 | 115 | TGF-β3-98-9 |
| | 58 | ACGTGCGTCATCACCGT | 17 | 116 | TGF-β3-98-10 |
| | 59 | CCAAGAAGCC | 10 | 117 | TGF-β3-98-11 |
| | 60 | CCTAATGCCTTCCA | 14 | 118 | TGF-β3-312 |
| | 61 | TCAGCAGGGCCAGG | 14 | 187 | GF-β-3rwk-1 |
| | 62 | GCAAAGTTCAGCAGGGC | 17 | 188 | GF-β-3rwk-2 |
| | 63 | GGCAAAGTTCAGCAGG | 16 | 189 | GF-β-3rwk-3 |
| | 64 | GTGGCAAAGTTCAGCAGG | 18 | 190 | GF-β-3rwk-4 |
| | 65 | GTGGCAAAGTTCAG | 14 | 191 | GF-β-3rwk-5 |
| | 66 | GACCGTGGCAAAGTTCAG | 18 | 192 | GF-β-3rwk-6 |
| | 67 | AGAGAGGCTGACCGT | 15 | 193 | GF-β-3rwk-7 |
| | 68 | GAGAGAGAGAGGCTGAC | 17 | 194 | GF-β-3rwk-8 |
| | 69 | ACAGAGAGAGGCTGA | 15 | 195 | GF-β-3rwk-9 |
| | 70 | GTGGACAGAGAGAGG | 15 | 196 | GF-β-3rwk-10 |
| | 71 | CAACTGGACAGAGAGAGG | 18 | 197 | GF-β-3rwk-11 |
| | 72 | TCTTCTTGATGTGGCC | 16 | 198 | GF-β-3rwk-12 |
| | 73 | CCCTCTTCTTCTTGATG | 17 | 199 | GF-β-3rwk-13 |
| | 74 | CACCCTCTTCTTCT | 14 | 200 | GF-β-3rwk-14 |
| | 75 | ATGGATTTCTTTGGCAT | 17 | 201 | GF-β-3rwk-15 |
| | 76 | GGATTTCTTTGGC | 13 | 202 | GF-β-3rwk-16 |
| | 77 | AAGTTGGACTCTCTTCTC | 18 | 203. | GF-β-3rwk-17 |
| | 78 | TAAGTTGGACTCTCTTCT | 18 | 204. | GF-β-3rwk-18 |
| PGE | 79 | TAGGAGTGGTTGAGGC | 16 | 1539 | Prostaglan.Rec.EP3-1 |
| | 80 | GTGTAGGAGTGGTTGAG | 17 | 1540 | Prostaglan.Rec.EP3-2 |
| | 81 | CTGTGTAGGAGTGG | 14 | 1541 | Prostaglan.Rec.EP3-3 |
| | 82 | CCCACATGCCTGTG | 14 | 1542 | Prostaglan.Rec.EP3-4 |
| | 83 | CGATGAACAACGAG | 14 | 1543 | Prostaglan.Rec.EP3-5 |
| | 84 | CTGGCGATGAACAACG | 16 | 1544 | Prostaglan.Rec.EP3-6 |
| | 85 | CGCTGGCGATGAAC | 14 | 1545 | Prostaglan.Rec.EP3-7 |
| | 86 | GAGCTAGTCCCGTTG | 15 | 1546 | Prostaglan.Rec.EP3-8 |
| | 87 | GCGAAGAGCTAGTCC | 15 | 1547 | Prostaglan.Rec.EP3-9 |
| | 88 | CCAGTTATGCGAAGAGC | 17 | 1548 | Prostaglan.Rec.EP3-10 |
| | 89 | CCCCAGTTATGCGAAG | 16 | 1549 | Prostaglan.Rec.EP3-11 |
| VEGF | 90 | CGGCCGCGGTGTGT | 14 | 119 | VEGF-98-1 |
| | 91 | CGGGAATGCTTCCGCCG | 17 | 120 | VEGF-98-2 |
| | 92 | CGGCTCACCGCCTCGGC | 17 | 121 | VEGF-98-3 |
| | 93 | CACGTCTGCGGATC | 14 | 122 | VEGF-98-4 |
| | 94 | CCCCGCATCGCATCAGGG | 18 | 123 | VEGF-98-5 |
| | 95 | CGCCTTGCAACGCG | 14 | 124 | VEGF-98-6 |
| | 96 | CCGACCGGGGCCGG | 14 | 125 | VEGF-98-7 |
| | 97 | GTTCATGGTTTCGG | 14 | 126 | VEGF-49 |
| | 98 | GCAGAAAGTTCATGG | 15 | 127 | VEGF-55 |
| | 99 | GCTGATAGACATCC | 14 | 128 | VEGF-188 |
| | 100 | GCGCTGATAGACAT | 14 | 129 | VEGF-190 |
| | 101 | GTAGCTGCGCTGATAG | 16 | 130 | VEGF-194 |
| | 102 | CTCGATCTCATCAG | 14 | 131 | VEGF-253 |
| | 103 | ATGTACTCGATCTCATC | 17 | 132 | VEGF-255 |
| | 104 | GAAGATGTACTCGATC | 16 | 133 | VEGF-260 |
| | 105 | CTTGAAGATGTACTCG | 16 | 134 | VEGF-263 |
| | 106 | GCATCGCATCAGGG | 14 | 135 | VEGF-292 |
| | 107 | CCGCATCGCATCAG | 14 | 136 | \IEGF-294 |
| | 108 | CATTTGTTGTGCTGTAGG | 18 | 137 | VEGF-422 |
| | 109 | GGTCTGCATTCACATTTG | 18 | 138 | VEGF-434 |
| | 110 | CTTTGGTCTGCATTC | 15 | 139 | VEGF-441 |
| | 111 | CTTTCTTTGGTCTGC | 15 | 140 | VEGF-445 |
| | 112 | GCTCTATCTTTCTTTGG | 17 | 141 | VEGF-450 |
| | 113 | GTCTTGCTCTATCTTTC | 17 | 142 | VEGF-455 |
| | 114 | CTTGTCTTGCTCTATC | 16 | 143 | VEGF-459 |
| | 115 | CATCTGCAAGTACGTTCG | 18 | 144 | VEGF-596 |
| | 116 | CACATCTGCAAGTACGTT | 18 | 145 | VEGF-598 |
| | 117 | GTCACATCTGCAAGTACG | 18 | 146 | VEGF-600 |
| | 118 | CATCTGCAAGTACG | 14 | 147 | VEGF-600-2 |
| | 119 | CACATCTGCAAGTAC | 15 | 148 | VEGF-601 |
| | 120 | GTCACATCTGCAAG | 14 | 149 | VEGF-604 |
| | 121 | CTTGTCACATCTGC | 14 | 150 | VEGF-607 |
| | 122 | GGCTTGTCACATCTGC | 16 | 151 | VEGF-607-2 |
| | 123 | CTCGGCTTGTCACATC | 16 | 152 | VEGF-610 |
| | 124 | CTCCTTCCTCCTGC | 14 | 153 | VEGF-638 |
| | 125 | GCTTGAAGATGTACCTCG | 16 | 154 | VEGF-766 |
| | 126 | CGTTGCTCTCCGACG | 15 | 155 | VEGF-r-1062 |
| IL-10 | 127 | GTAAAACTGGATCATCTC | 16 | 156 | U16720 |
| | 128 | CTTCTTTTGCAAGTCTGT | 18 | | |
| | 129 | TGAGCTGTGCATGCCTTC | 18 | | |
| | 130 | AGTCAGGAGGACCAG | 15 | | |
| | 131 | TGGGTGCCCTGGCCT | 15 | | |
| | 132 | CATGTTAGGCAGGTT | 15 | | |
| | 133 | AGGCATCTCGGAGATCT | 17 | | |
| | 134 | AAAGTCTTCACTCTGC | 16 | | |
| | 135 | AACAAGTTGTCCAGCTG | 17 | | |
| | 136 | GATCACCTCCTCCAG | 15 | | |
| | 137 | GGGTCTTCAGGTTCTCCC | 18 | | |
| | 138 | CACGGCCTTGCTCTTGTT | 18 | | |
| | 139 | TTATTAAAGGCATTCTTC | 18 | | |
| | 140 | AAGATGTCAAACTCACTC | 18 | | |
| | 141 | GTAGTTGATGAAGATGTC | 18 | | |
| | 142 | GATTTTGGAGACCTCT | 16 | | |
| | 143 | TCAGCTATCCCAGAGC | 16 | | |
| | 144 | GGCTGGGTCAGCTAT | 15 | | |
| | 145 | AAATCGTTCACAGAGAAG | 18 | | |
| | 146 | TCTTTCTAAATCGTTCAC | 18 | | |

1-29. (canceled)

30. A pharmaceutical composition comprising at least one TGF-beta antagonist, selected from the group consisting of
oligonucleotides hybridising with an area of the messenger RNA (mRNA) and/or DNA encoding TGF-beta, wherein the oligonucleotide comprises at least one of the sequences of SEQ NO: 1-78,
TGF-beta receptors and/or parts of them binding TGF-beta,
proteins, except antibodies, inhibiting TGF-beta
peptides of less than 100 kDa inhibiting TGF-beta
peptides being parts of TGF-beta
and at least one substance inhibiting cell proliferation and/or inducing cell death selected from the group consisting of temozolomide, nitrosoureas, Vinca alkaloids, antagonists of the purine and pyrimidine bases, cytostatic active antibiotics, caphthotecine derivatives, anti-androgens, anti-estrogens, anti-progesterones and analogs of gonadotropin releasing hormone.

31. The pharmaceutical composition of claim 30 wherein the at least one TGF-beta antagonist and the at least one substance inhibiting cell proliferation and/or inducing cell death are mixed together.

32. The pharmaceutical composition of claim 30 wherein the at least one TGF-beta antagonist and the at least one substance inhibiting cell proliferation and/or inducing cell death are separate.

33. The pharmaceutical composition according to claim 30 wherein at least one nucleotide of the oligonucleotide is modified at the sugar moiety, the base and/or the internucleotide linkage.

34. The pharmaceutical composition according to claim 33 wherein at least one modified internucleotide linkage is a phosphorothioate linkage.

35. The pharmaceutical composition according to claim 30 wherein
the nitrosourea is selected from the group of ACNU, BCNU and CCNU,
the Vinca alkaloid is selected from the group consisting of vinblastine, vincristine, and vindesine,
the antagonist of the purine and pyrimidine bases is selected from the group consisting of 5-fluorouracil, 5-fluorodeoxiuridine, cytarabine and gemcitabine,
the cytostatic antibiotic is selected from the group consisting of doxorubicin and liposomal PEGylated doxorubicin,
the camphthotecine derivative is selected from the group consisting of irinotecane and topotecane,
the anti estrogens are selected from the group consisting of tamoxifen, exemestane, anastrozole and fulvestraut,
the antiandrogens are selected from the group consisting of flutamide and bicalutamide,
the anti-progesterones are selected from the group consisting of mifepristone, and
the analogs of gonadotropin releasing hormone are selected from the group consisting of leuprolide and gosereline.

36. Method of treating a neoplasm comprising the step of administering the composition of claim 30 and optionally the step of applying radiation to a patient in need thereof.

37. Method of treating a neoplasm according to claim 36 wherein the neoplasm is selected from the group of bile duct carcinoma, bladder carcinoma, brain tumor, breast carcinoma, bronchogenic carcinoma, carcinoma of the kidney, cervical carcinoma, choriocarcinoma, cystadenocarcinome, embrional carcinoma, epithelial carcinoma, esophageal carcinoma, cervical carcinoma, colon carcinoma, colorectal carcinoma, endometrial carcinoma, gallbladder carcinoma, gastric carcinoma, head carcinoma, liver carcinoma, lung carcinoma, medullary carcinoma, neck carcinoma, non-small-cell bronchogenic/lung carcinoma, ovarian carcinoma, pancreas carcinoma, papillary carcinoma, papillary adenocarcinoma, prostata carcinoma, small intestine carcinoma, prostate carcinoma, rectal carcinoma, renal cell carcinoma, skin carcinoma, small-cell bronchogenic/lung carcinoma, squamous cell carcinoma, sebaceous gland carcinoma, testicular carcinoma, uterine carcinoma, acoustic neuromas, neurofibromas, trachomas, and pyogenic granulomas; pre-malignant tumors; rheumatoid arthritis; psoriasis; astracytoma, acoustic neuroma, blastoma, Ewing's tumor, astracytoma, craniopharyngloma, ependymoma, medulloblastoma, glioma, hemangloblastoma, Hodgkins-lymphoma, medullablastoma, leukaemia, mesothelioma, neuroblastoma, neurofibroma, non-Hodgkins lymphoma, pinealoma, retinoblastoma, retinoblastoma, sarcoma (including angiosarcoma, chondrosarcoma, endothelialsarcoma, fibrosarcoma, leiomyosarcoma, liposarcoma, lymphangioandotheliosarcoma, lyphangiosarcoma, melanoma, meningioma, myosarcoma, oligodendroglioma, osteogenic sarcoma, osteosarcoma), seminoma, trachomas, Wilm's tumor.

38. Method of treating a neoplasm according to claim 36 wherein the step of administering a pharmaceutical composition occurs before or after the step of applying radiation.

39. Method of treating a neoplasm according claim 36 wherein the step of administering a pharmaceutical composition occurs together with the step of applying radiation.

40. Method of treating a neoplasm according to claim 38 wherein the total amount of radiation within one cycle is from about 10 Gy to about 100 Gy.

41. Method of treating a neoplasm according to claim 40 wherein the total amount of radiation of one cycle is applied by several fractions from of about 1 Gy to about 2 Gy.
